(19) United States
(12) Patent Application Publication
(10) Pub. No.: US 2007/0269813 A1
(43) Pub. Date: Nov. 22, 2007

(54) METHODS AND ARRAYS FOR IDENTIFYING HUMAN MICROFLORA

(76) Inventors: Floyd E Dewhirst, Medfield, MA (US); Bruce Jay Paster, Hampton, NH (US); Susan K. Boches, Somerville, MA (US)

Correspondence Address: ANTOINETTE G. GIUGLIANO, P.C., 100 Cummings Center, Suite 342G, Beverly, MA, 01915, US

(21) Appl. No.: 11/556,296

(22) Filed: Nov. 3, 2006

Related U.S. Application Data
(60) Provisional application No. 60/733,023, filed on Nov. 3, 2005.

(51) Int. Cl. C07H 21/04; C12Q 1/68

(52) U.S. Cl. 435/6; 536/23.7; 536/24.32

(57) ABSTRACT

The present invention relates to a human microflora identification array. In particular, the present invention involves methods for identifying microorganisms, assessing microflora, diagnosing disease, providing a prognosis, and determining the efficacy of treatment, using one or more nucleic acid molecules having a sequence of SEQ ID NO: 1-585. The method includes obtaining a sample to be tested, and contacting nucleic acid molecules from the sample with the nucleic acid molecules of the present invention under conditions suitable for hybridization, and then detecting the complex formed by hybridization. The method can be carried out using an array. Hence, the present invention includes methods of identification of microorganism, methods for making such arrays, the arrays, and nucleic acid molecules used for same.

### RELATED APPLICATION

This application claims the benefit of U.S. Provisional Application No. 60/733,023, entitled “Methods and Arrays for Identifying Human Microflora,” filed Nov. 3, 2005.

The entire teachings of the above application are incorporated herein by reference.

### GOVERNMENT SUPPORT

The invention was supported, in whole or in part, by grants NIDCR DE-08303, NIDCR DE-10374, NIDCR DE-11443, NIDCR DE-12467-03S1 from National Institute of Dental and Craniofacial Research. The Government has certain rights in the invention.

### BACKGROUND OF THE INVENTION

Many microorganisms cause or worsen diseases that affect the human body. The basis for treatment of diseases often lies in the identification of the particular microorganism or set of microorganisms that is causing the condition. This is particularly applicable to diseases of the oral cavity. Certain methods currently used for the identification of microorganisms can take up to several days and do not always provide comprehensive results. In the case of the oral cavity, many methods that do identify microorganisms can only test for one microorganism or a small subset of microorganisms at a time. Such methods can be labor intensive, inefficient, and more importantly, they do not provide one with an assessment of the overall microfloral composition of the oral cavity.

Hence, a need exists for a test that effectively identifies microorganisms found on the human body, such as the oral cavity. In particular, a need exists to efficiently test for a number of microorganisms at one time to assess the composition of the microflora of the test site.

### SUMMARY OF THE INVENTION

The present invention relates to methods for identifying one or more microorganisms in a sample from an individual. The methods include contacting nucleic acid molecules obtained from the sample with one or more nucleic acid molecules (e.g., probes) having any one of SEQ ID NOs:1-295; a nucleic acid sequence having between about 80% and about 100% of contiguous nucleotides of any one of SEQ ID NO: 1-295; any one of SEQ ID NOs:296-585; a nucleic acid sequence having between about 15 and about 25 contiguous nucleotides of any one of SEQ ID NO: 296-585; a reverse complement thereof; and any combination thereof. The contacting step is performed under conditions suitable for hybridization to thereby form a complex. Molecules obtained from the sample include nucleic acid sequences that are copied or amplified, as further described herein. The methods further include detecting the presence or absence of the complex, wherein the presence of the complex indicates the presence of one or more microorganisms, as defined herein, in the sample and the absence of the complex indicates the absence of one or more microorganisms in the sample. “Absence” is referred to herein as an amount of a complex that is below a detectable level or limit. Microorganisms identified by SEQ ID NOs: 1-585 include at least one of the following:
Actinobacillus actinomycetemcomitans, Actinobaculum sp. EL030, Actinomyces georgiae, Actinomyces gerensceriae, Actinomyces naeslundii I, Actinomyces naeslundii II, Actinomyces odontolyticus, Actinomyces sp. AP064, Actinomyces sp. B19SC, Actinomyces sp. B27SC, Actinomyces sp. EP005, Actinomyces sp. EP011, Actinomyces sp. EP053, Actinomyces israelii, Atopobium parvulum, Atopobium rimae, Atopobium sp. C019, Tannerella forsythia, Tannerella forsythia, Bacteroidetes sp. _X083, Bacteroidetes sp. AU126, Bifidobacterium (genus-specific), Bifidobacterium dentium, Bifidobacterium sp. strain A32ED, Bifidobacterium sp. CX010, Brevundimonas diminuta, Bulledia extructa/Solobacterium moorei, Campylobacter concisus, Campylobacter gracilis, Campylobacter rectus/concisus, Campylobacter cluster: (C. rectus/showae/curvus), Campylobacter showae, Capnocytophaga ochracea/sp. BB167, Capnocytophaga sp. _X066, Capnocytophaga sp. _X089, Capnocytophaga sp. AA032, Capnocytophaga sp. BB167, Capnocytophaga cluster: (C. ochracea/sp. BM058/BU084/DZ074/BR085, Capnocytophaga sp. BR085, Capnocytophaga sp. DS022, Capnocytophaga gingivalis/sp. S3, Capnocytophaga sputigena, Cardiobacterium hominis, Corynebacterium durum, Corynebacterium matruchotii, Cryptobacterium curtum, Desulfobulbus sp. _R004/CH031, Dialister invisus, Dialister pneumosintes, Eikenella corrodens, Eubacterium brachy, Eubacterium infirmum, Eubacterium nodatum, Eubacterium sp. IR009, Eubacterium saphenum, Eubacterium sp. strain A3MT, Eubacterium sp. BB124, Eubacterium sp. BB142, Eubacterium sp. D008, Eubacterium sulci, Eubacterium yurii, Filifactor alocis, Fusobacterium sp. _I035, Fusobacterium cluster: (F. nucleatum/CZ006/_R002/ss. vincentii/naviforme), Fusobacterium nucleatum ss. nucleatum, Fusobacterium nucleatum ss. polymorphum, Fusobacterium periodonticum, Fusobacterium sp. BS011, Gemella haemolysans, Gemella morbillorum, Granulicatella adicens/elegans, Haemophilus influenzae, Haemophilus parainfluenzae/paraphrophilus, Haemophilus paraphrophaemolyticus/sp. BJ021, Haemophilus segnis, Haemophilus sp. BJ095, Kingella denitrificans, Kingella oralis, Lactobacillus casei/rhamnosis/zeae, Lactobacillus fermentum, Lactobacillus gasseri, Lactobacillus sp. CX036 vaginalis, Lactobacillus sp. HT070, Lautropia mirabilis, Lautropia sp. AP009, Leptotrichia buccalis, Leptotrichia hofstadii, Leptotrichia sp. DR011, Leptotrichia sp. FB074/BB002, Leptotrichia sp. GT018, Leptotrichia wadei, Megasphaera sp. BB166, Megasphaera sp. BU057, Megasphaera sp. CS025, Micromonas micros, Micromonas cluster: M. micros/FG014/BS044, Micromonas sp. DA014, Mycoplasma faucium, Mycoplasma hominis, Mycoplasma salivarium, Neisseria elongata, Neisseria cluster I: N. elongata/sp. AP015/Eikenella corrodens, Neisseria flavescens, Neisseria cluster II: (N. mucosa/sicca/flava/AP015), Neisseria pharyngis, Neisseria cluster III: (N. polysaccharea/gonorrhoeae/meningitides), Neisseria bacilliformis/sp. AP132, Neisseria mucosa/sp. AP060, Neisseria sp. strain B33KA, Olsenella genomospecies C1, Peptostreptococcus sp. CK035, Porphyromonas catoniae, Porphyromonas endodontalis cluster: (P. endodontalis/F016/BB134/AJ002), Porphyromonas gingivalis, Porphyromonas sp. BB134, Porphyromonas cluster: (sp. BR037/DP023/EP003), Porphyromonas sp. CW034/DS033, Porphyromonas sp. CW034/DS033, Porphyromonas sp. DP023, Prevotella buccae, Prevotella (Bacteroides) heparinolytica, Prevotella intermedia, Prevotella loeschii/GU027, Prevotella cluster I: (P. loeschii/GU027/B31FD, Prevotella melaminogenica, Prevotella nigrescens, Prevotella oralis, Prevotella oris/_F045, Prevotella oulora, Prevotella pallens, Prevotella cluster II: (P. denticola/sp. AH005/AO036), Prevotella denticola/sp. AH005, Prevotella sp. AH125, Prevotella sp. BE073, Prevotella sp. BI027, Prevotella sp. CY006/FL019, Prevotella sp. DO027, Prevotella sp. DO039, Prevotella sp. DO045, Prevotella sp. DO022, Prevotella sp. FM005, Prevotella sp. HF050, Prevotella tannerae, Propionibacterium acnes, Propionibacterium sp. strain FMA5, Pseudomonas aeruginosa, Rhodocyclus sp. strain A08KA, Rothia dentocariosa, Rothia dentocariosa/mucilaginosa, Selenomonas dianae, Selenomonas flueggii, Selenomonas infelix, Selenomonas noxia, Selenomonas sp. AA024, Selenomonas sp. AH132, Selenomonas sp. AJ036, Selenomonas sp. CI002, Selenomonas sp. CS002, Selenomonas sp. CS015, Selenomonas sp. CS024, Selenomonas sp. DD020, Selenomonas sp. DM071, Selenomonas sp. EZ011, Selenomonas sp. DS051, Selenomonas sp. EW076, Selenomonas sp. EW079/JS031, Selenomonas sp. EW084/DS071, Selenomonas sputigena, Streptococcus (genus-specific), Streptococcus anginosus/gordonii, Streptococcus anginosus/intermedius, Streptococcus constellatus/intermedius, Streptococcus cristatus, Streptococcus cluster I: (S. gordonii/anginosus/mitis, Streptococcus infantis/sp.FN042, Streptococcus mitis biovar 2, Streptococcus cluster II: (S. mitis/oralis/pneumoniae), Streptococcus mutans, Streptococcus parasanguinis, Streptococcus salivarius, Streptococcus cluster III: (S. sanguinis/salivarius/mitis/C3, Streptococcus australis, Streptococcus cluster IV: sp. C6/C3/P4/7A, Stretococcus sobrinus, Synergistes (Phylum-specific, Synergistes sp. _D084, Synergistes sp. _W028, Synergistes sp. _W090, Synergistes sp. BB062, Synergistes sp. BH017, Tannerella sp. BU063, TM7 sp. _I025, TM7 sp. AH040, TM7 sp. BE109, TM7 sp. BE109/BU080, Treponema 08:A:pectinovorum, Treponema (genus specific), Treponema denticola, Treponema lecithinolyticum, Treponema medium, Treponema socranskii (all subspecies), Treponema sp. AT039, Treponema vincentii, Veillonella dispar/_X 042/, Veillonella (genus-specific), Veillonella atypica, Veillonella parvula, Veillonella sp. AA050/_X042, Veillonella sp. BU083, and Escherichia coli.
   Table 1, shown in FIG. 4A-R, provides the correlation of probes to microorganisms. The present invention includes embodiments in which one, two or three different probes identify a microorganism (e.g., a single microorganism, closely related microorganisms, or genera, as further described herein). The sample from the individual can be obtained from the oral cavity and/or contiguous cavities such as the sinus, esophagus, respiratory tract, lungs, pharynx, eustachian tube, and middle ear. Samples can also be obtained from extraoral areas of the human body, such as the vagina, blood, pus, spinal fluid, and gastrointestinal tract. The nucleic acid molecules having SEQ ID NOs: 1-585 were designed based on the sequence of 16S rRNA of cultivable organisms or the 16S rRNA sequences of clone libraries. Hence, the methods described herein include detecting nucleic acid molecules in the sample, specifically bacterial 16S rRNA or 16S rRNA genes. The methods include labeling the nucleic acid molecules of the sample with a detectable label (e.g., fluorescent dyes, streptavidin conjugate, magnetic beads, dendrimers, radiolabels, enzymes, calorimetric labels, nanoparticles, and/or nanocrystals). In a preferred embodiment, the nucleic acid molecules of the present invention are bound in an array format to a solid support, such glass, silica chips, nylon membrane, polymer, plastic, ceramic, and metal. Optical fiber can also be coated with probes, or probes are infused into a gel or matrix is that put on a solid support.

The methods of the present invention include assessing the compositional flora of microorganisms in an individual. The methods include contacting nucleic acid molecules obtained from a sample from the individual with one or more nucleic acid molecules having a nucleic acid sequence of SEQ ID NOs:1-585 (e.g., one or more of each individual sequence, or any combination thereof), a nucleic acid sequence having between about 80% and about 100% of contiguous nucleotides of any one of SEQ ID NO: 1-295; a nucleic acid sequence having between about 15 and about 25 contiguous nucleotides of any one of SEQ ID NO: 296-585; a reverse complement thereof, and any combination thereof. This is done under conditions suitable for hybridization between the nucleic acid molecules from the sample and the nucleic acid molecule to thereby form a complex; and then the method involves detecting the presence or absence of the complex. The presence of the complex indicates the presence of the microorganism in the sample and the absence of the complex indicates the absence of the microorganism in the sample. The compositional flora is composed of the presence, absence, or both of one or more microorganisms.

The present invention further includes monitoring the effect of an oral product on the microflora of an individual. The method includes determining the presence, absence, level or percentage of nucleic acid molecules that hybridize to the nucleic acid molecules of the present invention, as described herein, at one or more time points, and comparing these determinations. Oral products that are used by the present invention include toothpaste, mouthwash, fluoride, breath enhancers, tooth-whitening treatments, floss, and the like. Embodiments of the present invention include assessing an individual with or without a particular oral disease or condition (e.g., sensitive teeth, gum disease, cavities, abscesses, plaque buildup, halitosis, cold sores).

The present invention further includes methods for diagnosing an individual with a disease or condition, or providing a prognosis of a disease or condition. Although the probes of the present invention identify microorganisms; the presence, absence, or level of the 16S rRNA from microorganisms in a sample can be directly correlated with a disease or condition. Hence, the methods of the present invention include determining the presence, absence (e.g., below a detectable level), level or percentage of one or more nucleic acid molecules from the sample of an individual that hybridize to the nucleic acid molecules of the present invention. The presence, absence, level or percentage of one or more complexes indicates, in one embodiment, the presence, absence or severity of the disease or condition. In another embodiment, the present invention involves identification of microorganisms for purposes of diagnosis or providing a prognosis. The steps include contacting nucleic acid molecules from a sample from the individual with one or more nucleic acid molecules of the present invention under conditions suitable for hybridization to thereby form a complex; and detecting the presence or absence of the complex. In one aspect of the invention, the presence of the complex indicates the presence of the microorganism in the sample and the absence of the complex indicates the absence of the microorganism in the sample. The method also includes determining the disease associated with the presence, absence, or both of one or more microorganisms (e.g., by assessing the microfloral composition). Examples of diseases of the oral cavity include periodontal disease, alveolar osteoitis (e.g., dry socket), caries (e.g., tooth decay), and oral cancer. Additional diseases that are encompassed by the present invention include extraoral diseases, such as diabetes, AIDS, Sjögren's syndrome, smoking and alcoholism.

Similarly, the present invention embodies methods for monitoring treatment or efficacy of therapy for an individual. The steps of the method involve determining the presence, absence, level or percentage of nucleic acid molecules present in a sample from the individual as described herein, e.g., at one or more time points; and comparing these determinations. The comparison or analysis indicates the efficacy of therapy. Examples of therapy include antibiotic therapy, surgery, and administration of medication.

The methods of the invention include identifying one or more unnamed or uncultivated microorganisms by contacting nucleic acid molecules obtained from the sample with one or more nucleic acid molecules of the present invention under conditions suitable for hybridization to thereby form a complex, and detecting the complex. The nucleic acid molecules (e.g., the probes) have a sequence that is complementary to a non-conserved region of nucleic acid sequence from the unnamed or uncultivated microorganism (e.g., known from strains or 16S rRNA clones).

Another aspect of the invention pertains to methods for determining the presence or absence of one or more of the microorganisms in accordance with Table 1. The method includes detecting the presence of one or more nucleic acid molecules having a nucleic acid sequence, as described herein. Table 1 shows the correlation of the sequence to the microorganism being identified.

In particular, the methods of the present invention for identifying a microorganism in a sample from an individual include amplifying and labeling DNA from the sample with a detectable label using a Polymerase Chain Reaction (PCR); contacting the labeled DNA from the sample with one or more nucleic acid molecules that are arranged in an array and have a nucleic acid sequence, as described herein, under high stringency conditions, to thereby form a complex between the labeled DNA from the sample and the nucleic acid molecule. The method further includes detecting the presence or absence of the complex. The presence of a complex indicates the presence of the microorganism and the absence of a complex indicates the absence of the microorganism. The microorganisms are identified by SEQ ID NOs: 1-585 in accordance with Table 1.

In another aspect, the present invention includes methods for identifying a microorganism in a sample from an individual, by reverse transcribing RNA obtained from the sample to thereby obtain DNA; labeling the DNA; contacting the DNA from the sample with one or more nucleic acid molecules of the present invention as described herein under high stringency conditions suitable for hybridization to thereby form a complex. Alternatively, the method does not include a step of reverse transcription. RNA (e.g., rRNA) can be obtained directly from the sample and labeled, e.g., by hybridizing universal probes to a portion of the RNA. The labeled RNA is then contacted with the one or more nucleic acid molecules of the present invention. The molecules of the present invention (e.g., the probes that identify the microorganism) hybridize to a different portion of the RNA than the labeled universal probes. The methods of the present invention further encompass detecting the presence or absence of the complex. The presence of the complex indicates the presence of the microorganism in the sample and the absence of the complex indicates the absence of the microorganism in the sample. Microorganisms identified by SEQ ID Nos: 1-585 are described herein.

The present invention embodies arrays for the identification of one or more microorganisms. The array of the present invention includes one or more nucleic acid molecules having a nucleic acid sequence of the present invention. The array or any method described herein includes use of each or all of the 585 sequences, or any number, percentage or combination thereof. Each molecule is bound to the surface of a solid support in a different localized area to form the array. The solid support can be made from glass, silica chips, nylon membrane, polymer, plastic, ceramic, metal, coated on optical fiber. The probes can also be infused into a gel or matrix is that put on a solid support. The present invention, in an embodiment, includes between about 1 and about 8 different arrays on a single solid support, and preferably about 5 different arrays. Additionally, the same array can be duplicated 2 or more times, and preferably about 3 times. In yet another embodiment, the array of the present invention used for the identification of one or more microorganisms, includes at least about 10% (e.g., about 20%, 30%, 40%, 50%, 60%, 70%, 80%, 90%, or 100%) of the nucleic acid molecules of the present invention, or at least about 20 (e.g., about 40, 60, 80, 100, 120, 140, 160, 180, 200, 220, 240, 260, 280, 300, 320, 340, 360, 380, 400, 420, 440, 460, 480, 500, 520, 540, 560 or 580) nucleic acid molecules of the present invention.

The present invention also encompasses kits. The kits include arrays, as described herein, and reagents used for carrying out a nucleic acid hybridization assay. Such reagents include compounds used to detect hybridization; unlabeled primers, labeled primers, washing solutions; and buffers.

In another aspect, the invention relates to isolated nucleic acid molecules (e.g., RNA or DNA) from bacteria isolated from a human oral cavity, sinus, esophagus, respiratory tract, lungs, pharynx, eustachian tube, or middle ear. The nucleic acid molecules have one or more the sequences of the present invention, as described herein. The nucleic acid molecules identify the microorganisms described herein, and as shown in Table 1.

The invention pertains to methods for making an array for the identification of a microorganism. The method includes attaching to a solid support one or more nucleic acid molecules described herein to the surface of a solid support in a different localized area. Microorganisms that are identified by SEQ ID Nos: 1-585 are described herein. The methods include attaching the nucleic acid molecules in a solution having a concentration of between about 30 μM and 200 μM (e.g., about 100 μM). The method, in an embodiment, includes arranging between about 1 and about 8 arrays on a solid support, as described herein, and duplicating the same array 2 or more times (e.g., about 3 times). The method can further include synthesizing a capture probe having a nucleic acid sequence as described herein prior to attaching the probe to the solid support, also as described herein. The method can further include attaching a spacer to the probe (e.g., using a plurality of thymidine bases), and/or attaching a linking molecule to the probe that reacts with the solid support (e.g., an amine group). Methods of making the array of the present invention include inserting, growing, or integrating on a solid support or within a gel matrix one or more nucleic acid molecules described herein in a different localized area.

Advantages of the present invention include the ability to test for multiple microorganisms at one time, under the same conditions. The present invention allows for efficient and accurate identification of microfloral composition, in particular for the oral and related cavities. Additionally, the development of a microarray allows for microbe identification in a relatively short period of time, (e.g., 30 seconds-36 hours). Such a tool allows dentists and doctors to more effectively diagnose and treat individuals having diseases associated with or caused by one or more microorganisms.

### BRIEF DESCRIPTION OF THE DRAWINGS

FIG. 1 is a schematic showing one example of a human microflora identification microarray on a slide with 5 independent hybridization sections, comprised of 3 replica 64-element microarrays. Each hybridization section can be covered by a 12×18 mm cover slip.

FIG. 2 is a photograph of a microarray showing hybridization to a specific probe and 4-corner universal probes.

FIG. 3A is a photograph of a microarray showing hybridization with DNA from a healthy subject panel.

FIG. 3B is a photograph of a microarray showing hybridization with DNA from a periodontitis subject panel.

FIGS. 4A-4R include Table 1 showing a list of capture probes and the corresponding microorganism that is identified by that probe, and the immediate flanking sequence of the capture probes and the corresponding microorganism that is identified by that probe. Specifically, the microorganism (probe target) Genbank Accession number, Probe ID, Probe Sequence, and Probe Sequence Extended (probe with flanking regions), and sequence identifier.

FIG. 5 includes Table 2 which provides a list of species or phylotypes associated with diseased and healthy samples.

FIGS. 6A and 6B are schematics showing the process of detecting rRNA in a sample by directly labeling and assessing rRNAs levels, for example, in a dental chair-side diagnostic kit.

FIG. 7 is a photograph showing an image of the low resolution initial scan of an entire slide, showing five individual arrays printed on the slide.

FIG. 8A is a schematic of the reproducibility between the 2 sub-arrays for each whole array (2 subarrays underneath 1 coverslip), along with a table of the bacteria grown on individual plates.

FIGS. 8B-C is a photograph of a high-resolution scan of 5 individual arrays, each hybridized with labeled product from the same starting template (amplified as 5 separate reactions).

FIG. 9A-C is a table comparing median intensity scores (with background intensity subtraction) for the spots across all 5 individual arrays within 1 slide.

FIG. 10 is a graph of the mean intensity values (log2) of various probes of 1 sample hybridized on 5 arrays within 1 slide.

FIG. 11A is a schematic of the reproducibility between 2 individual arrays hybridized on different slides, along with a table of the bacteria grown on individual plates.

FIG. 11B is a photograph of a high-resolution scan of 2 individual arrays hybridized on different slides with labeled DNA from the same starting template (amplified as 2 separate reactions).

FIG. 12 is a table comparing median intensity scores (with background subtraction) for the 2 individual arrays hybridized on different slides.

FIG. 13 is a graph of the mean intensity values (log2) of various probes of the same DNA hybridized on 2 different slides.

### DETAILED DESCRIPTION OF THE INVENTION

A description of preferred embodiments of the invention follows.

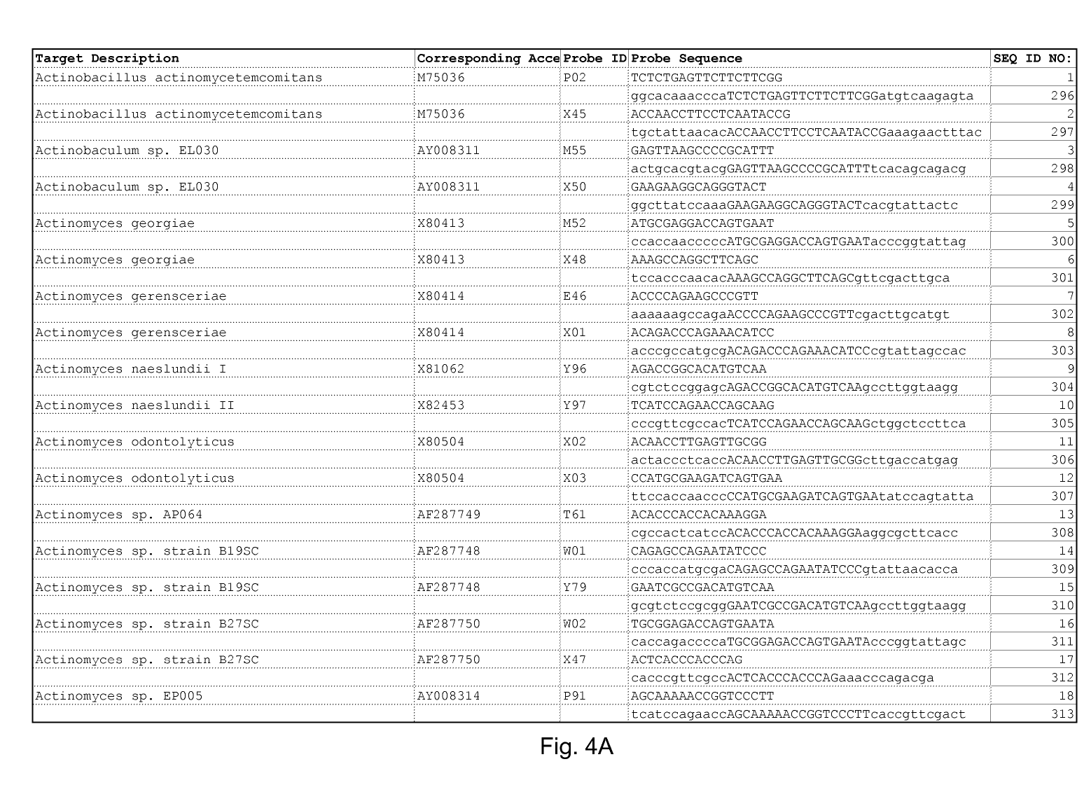

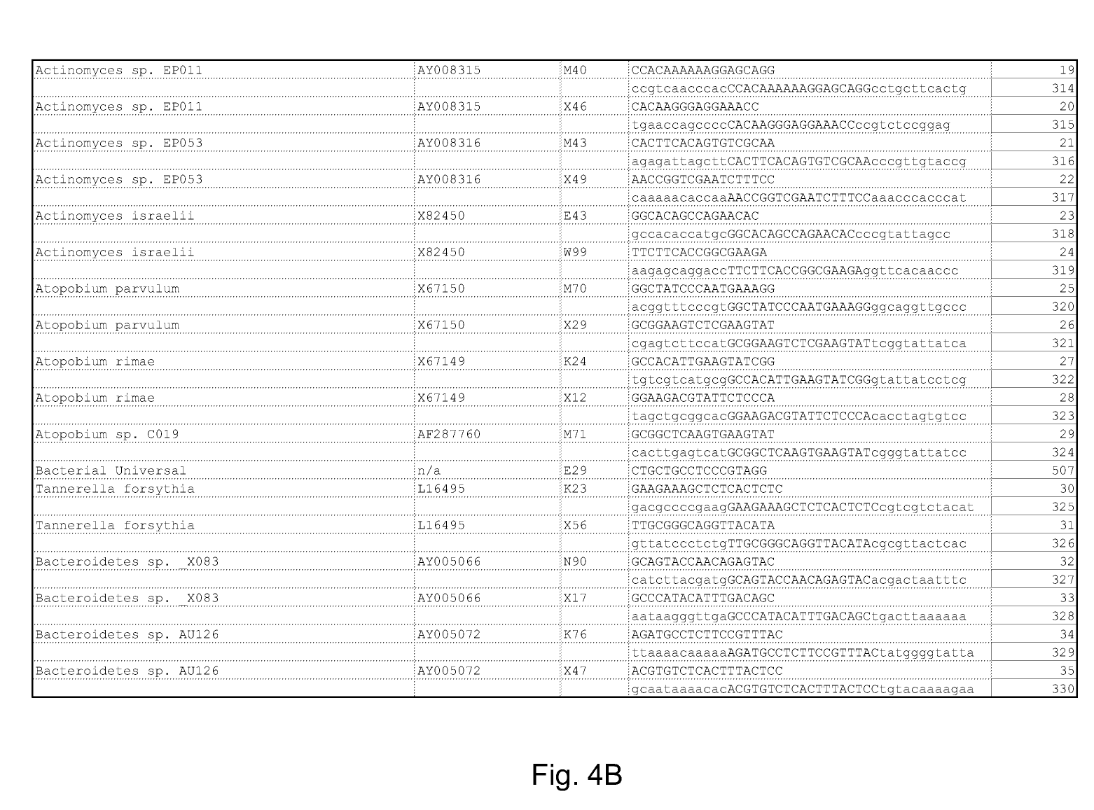

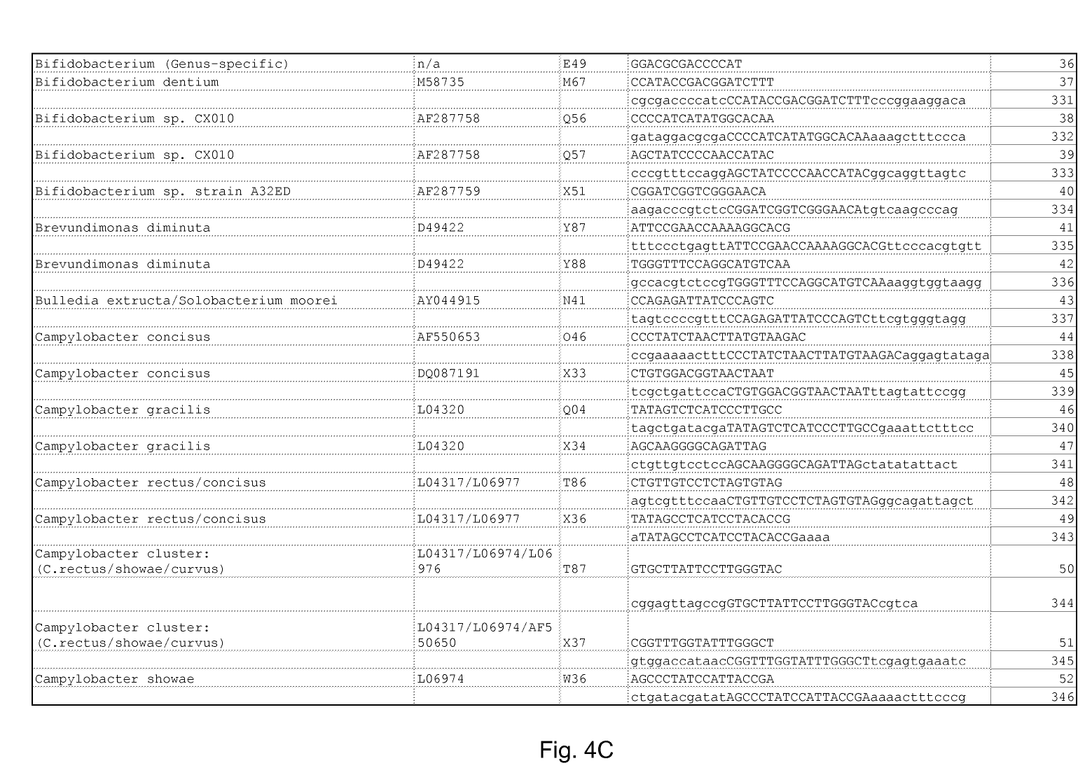

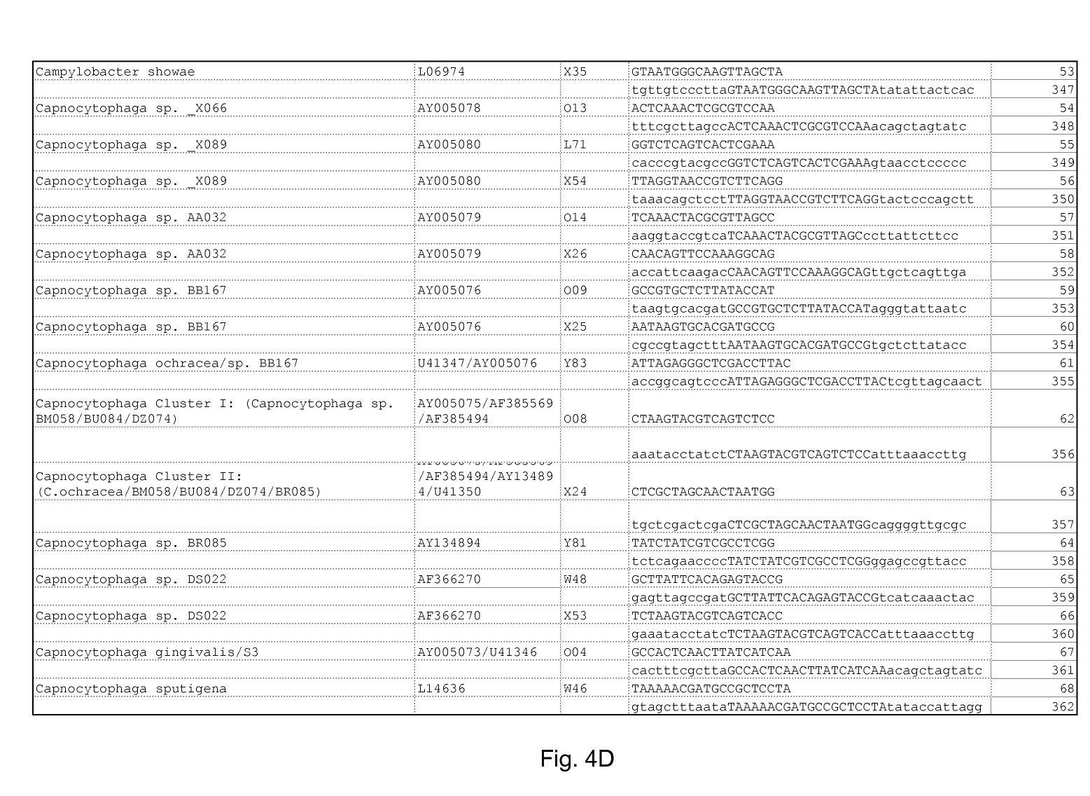

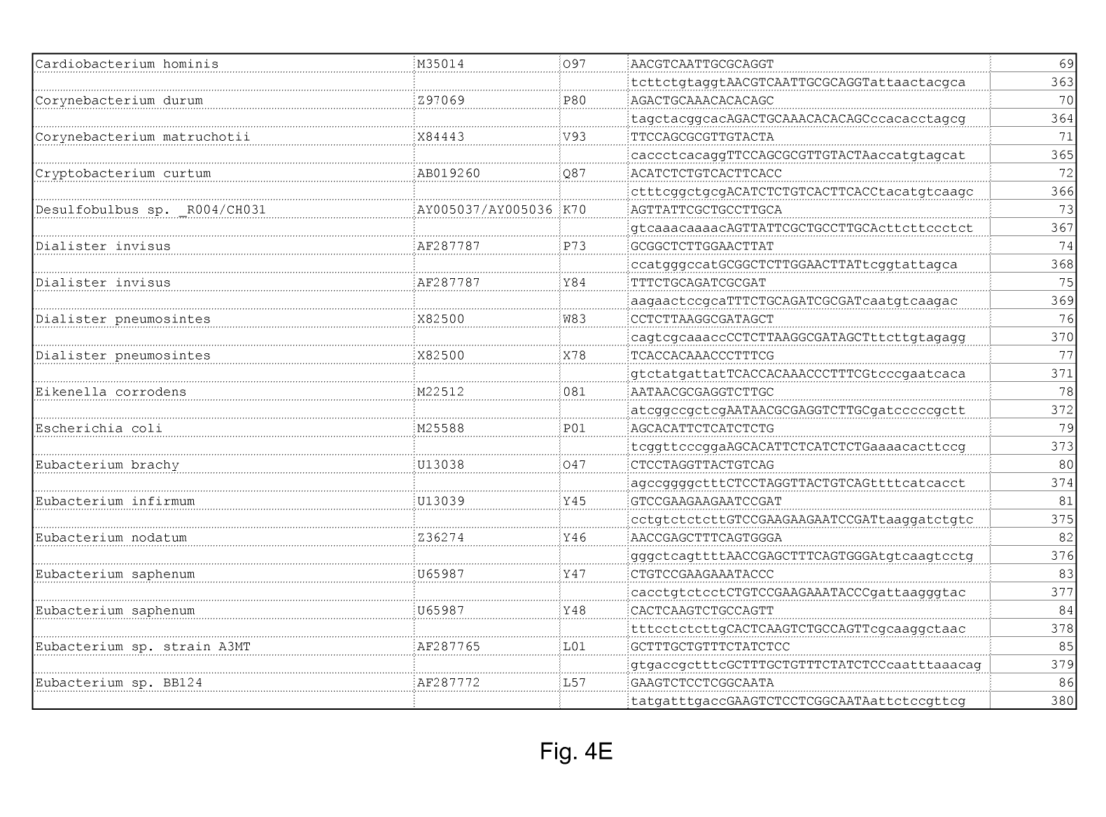

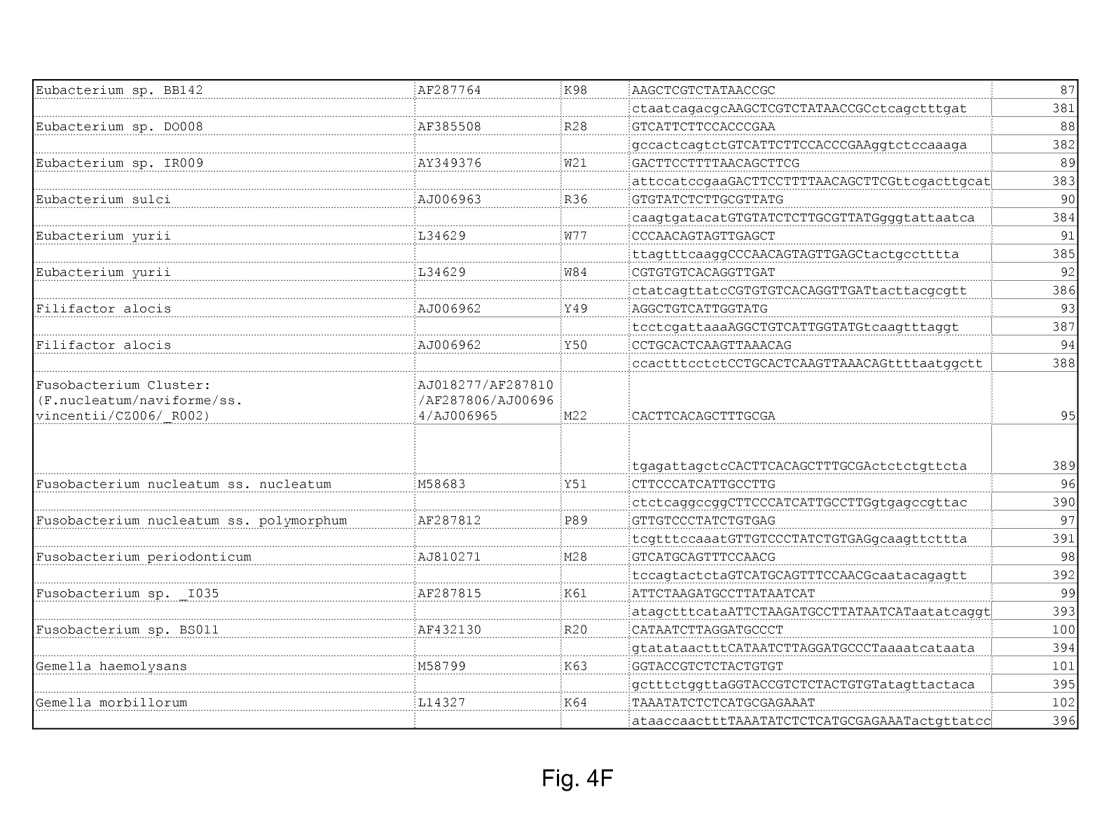

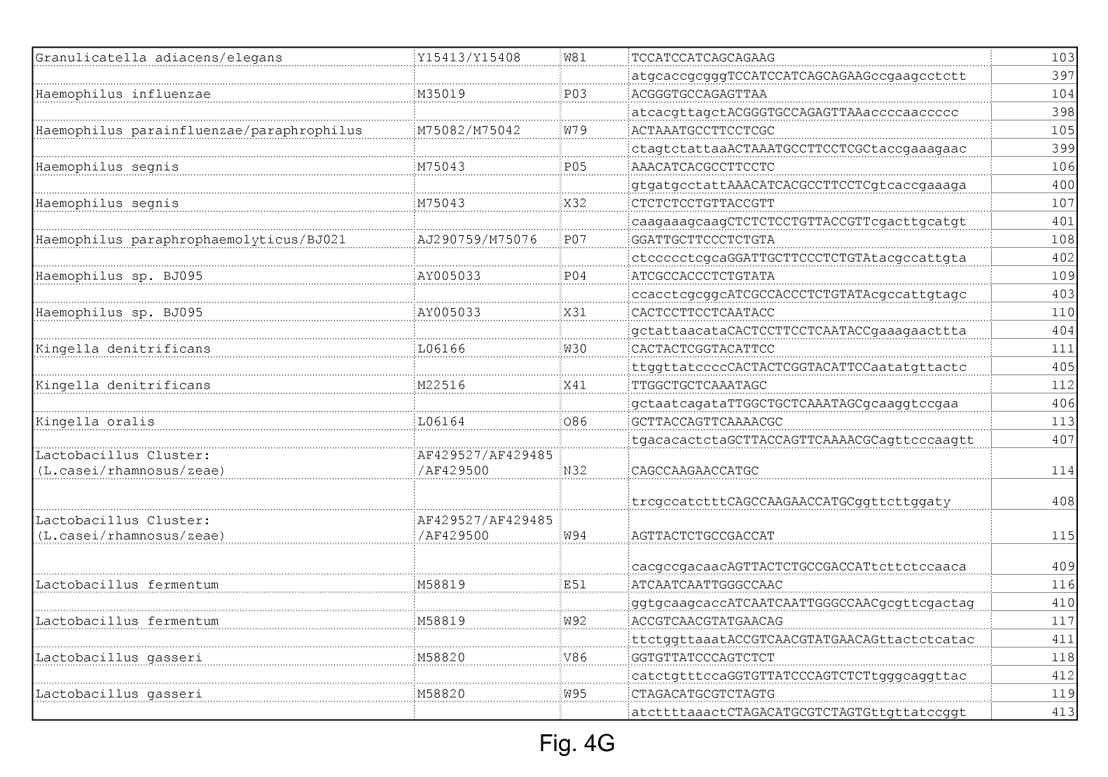

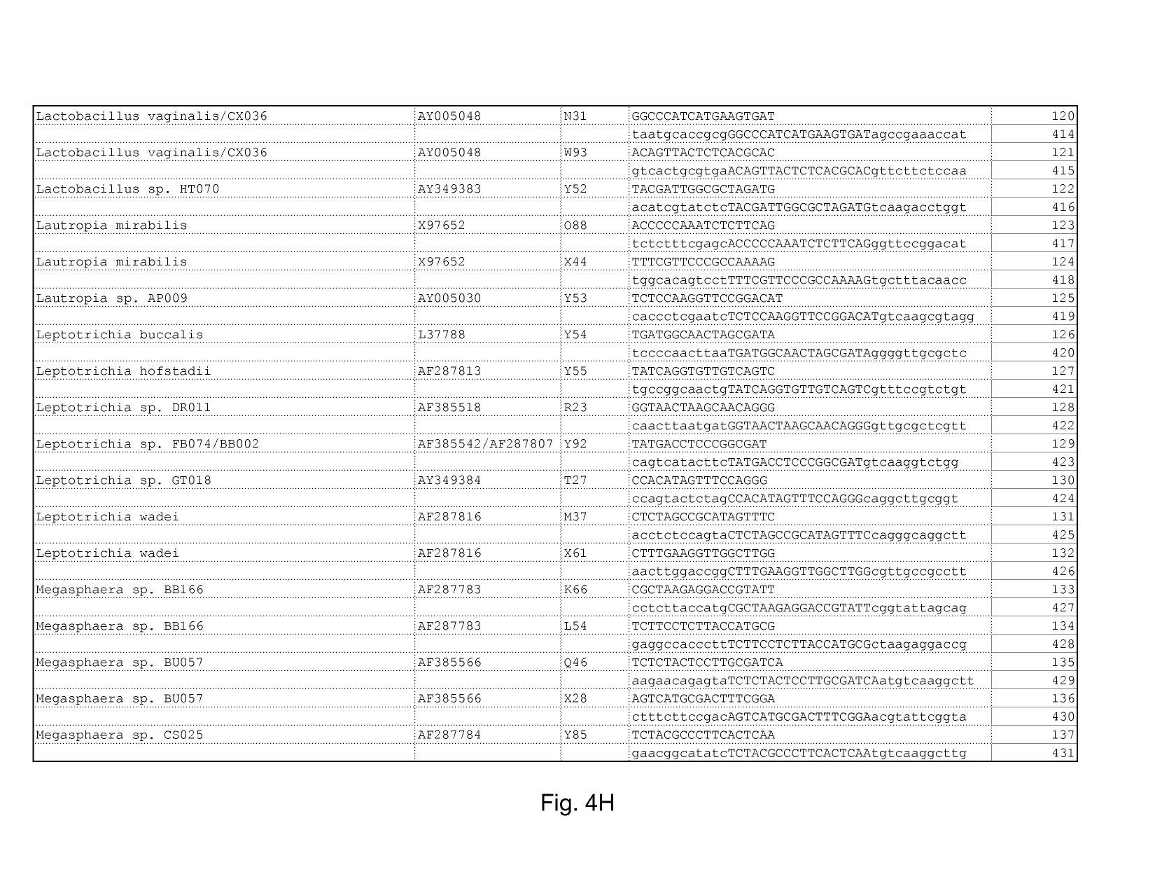

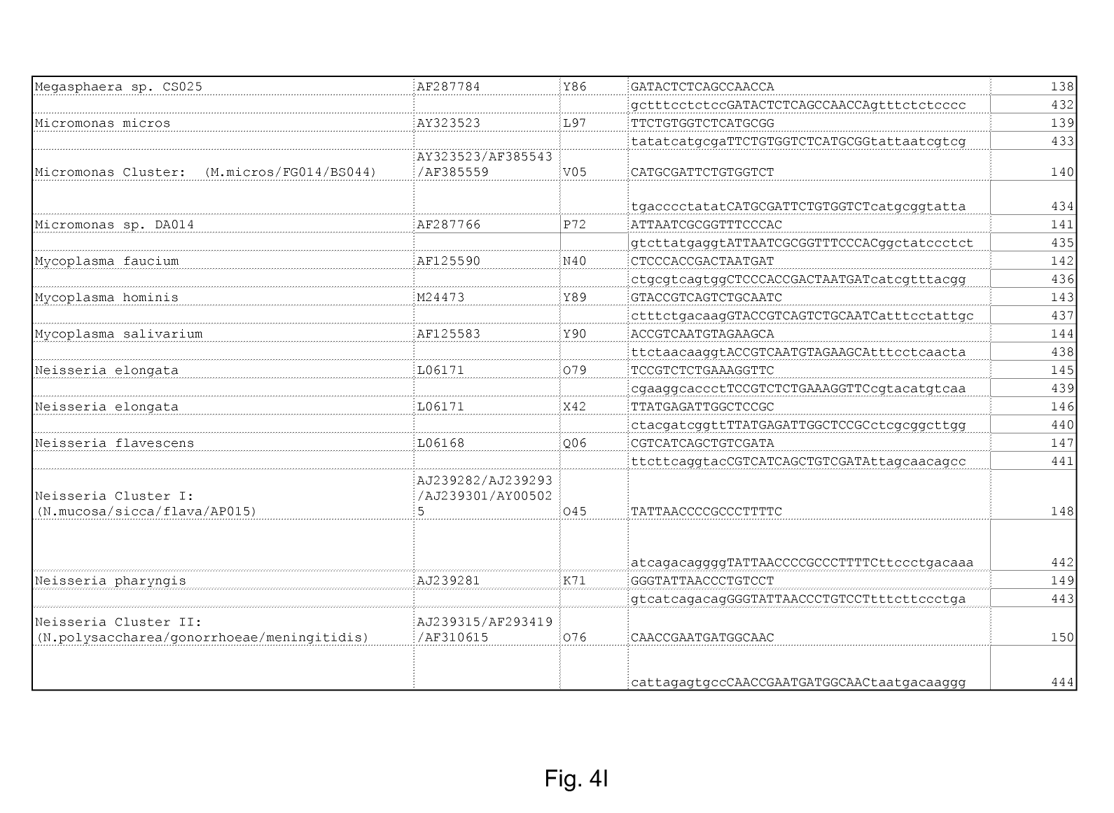

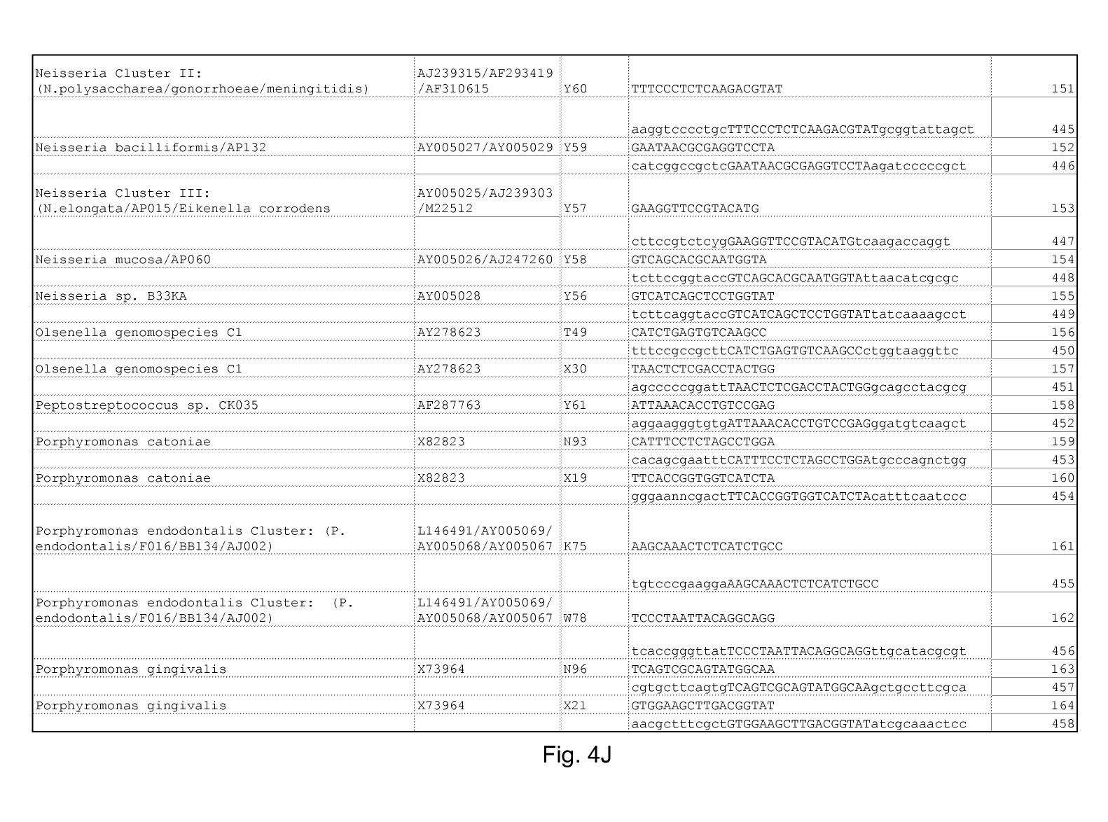

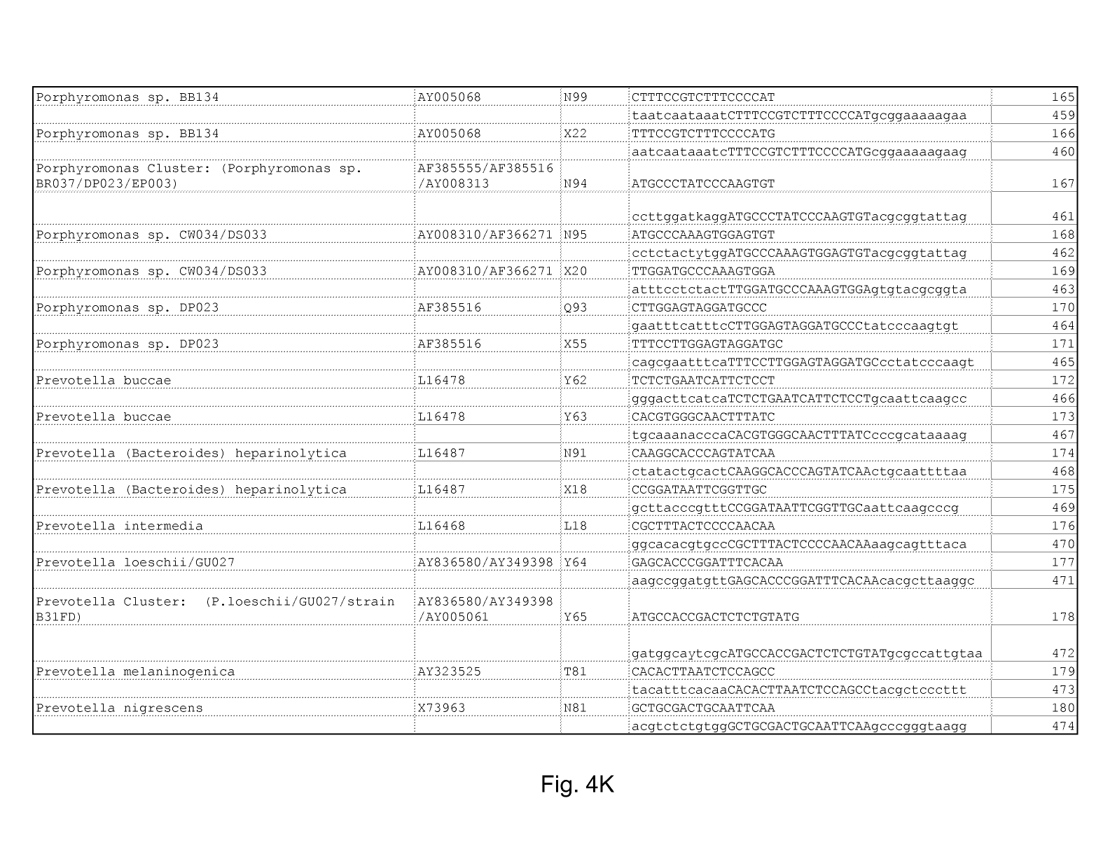

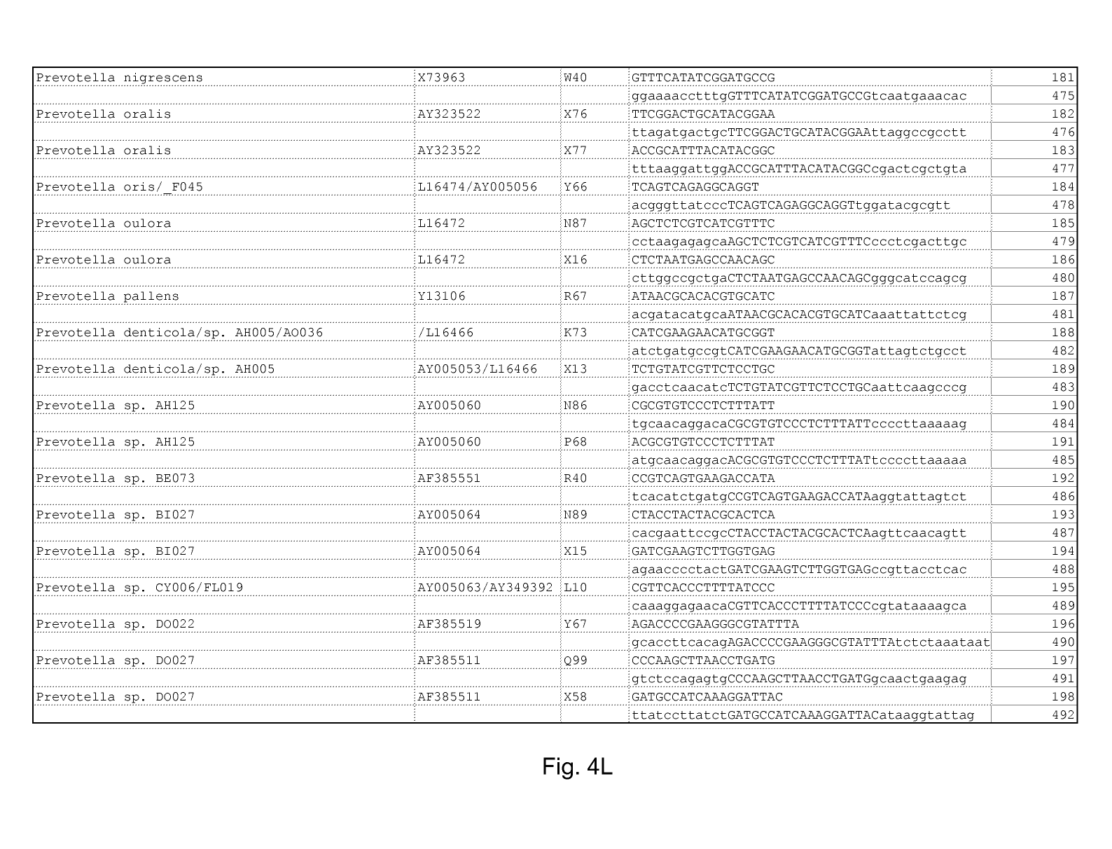

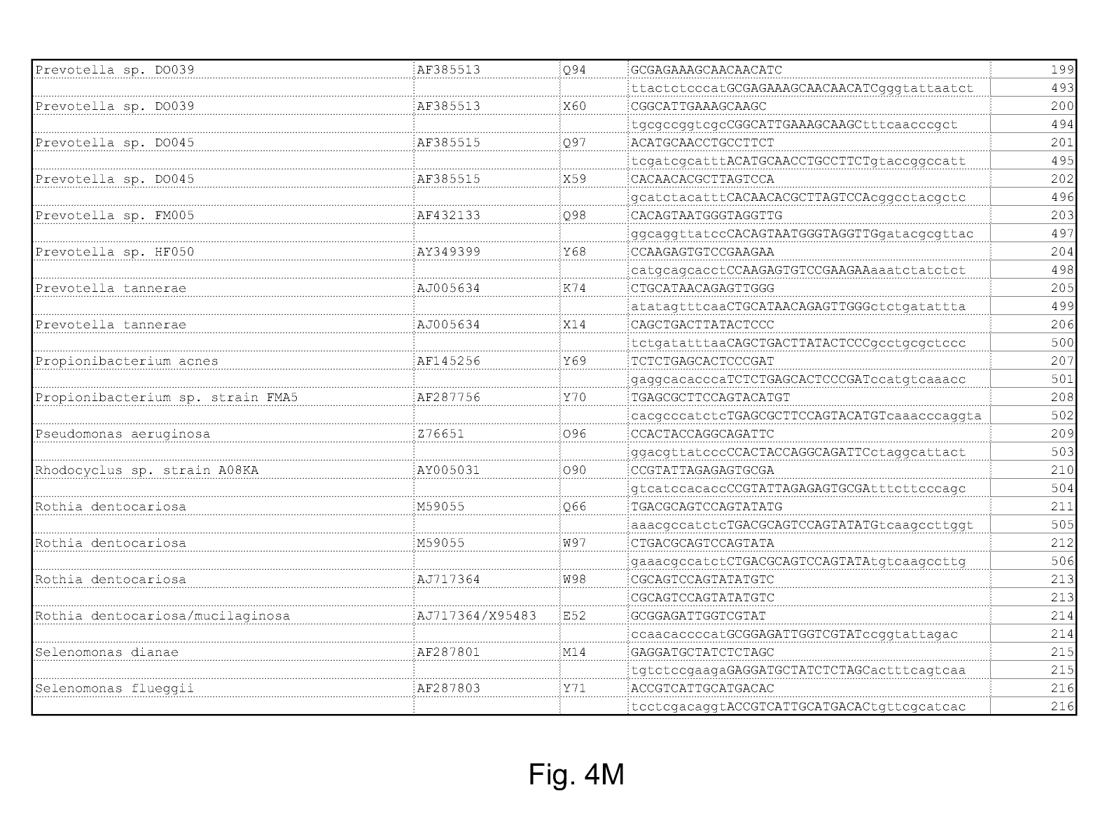

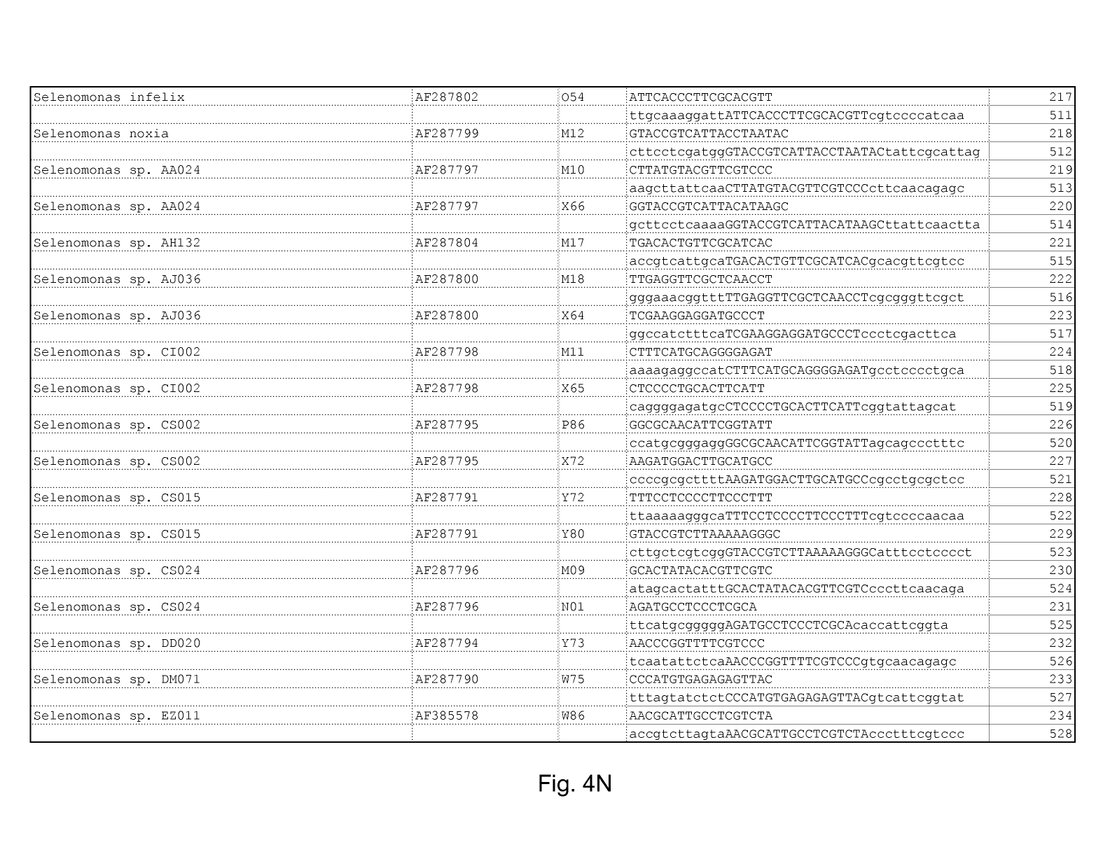

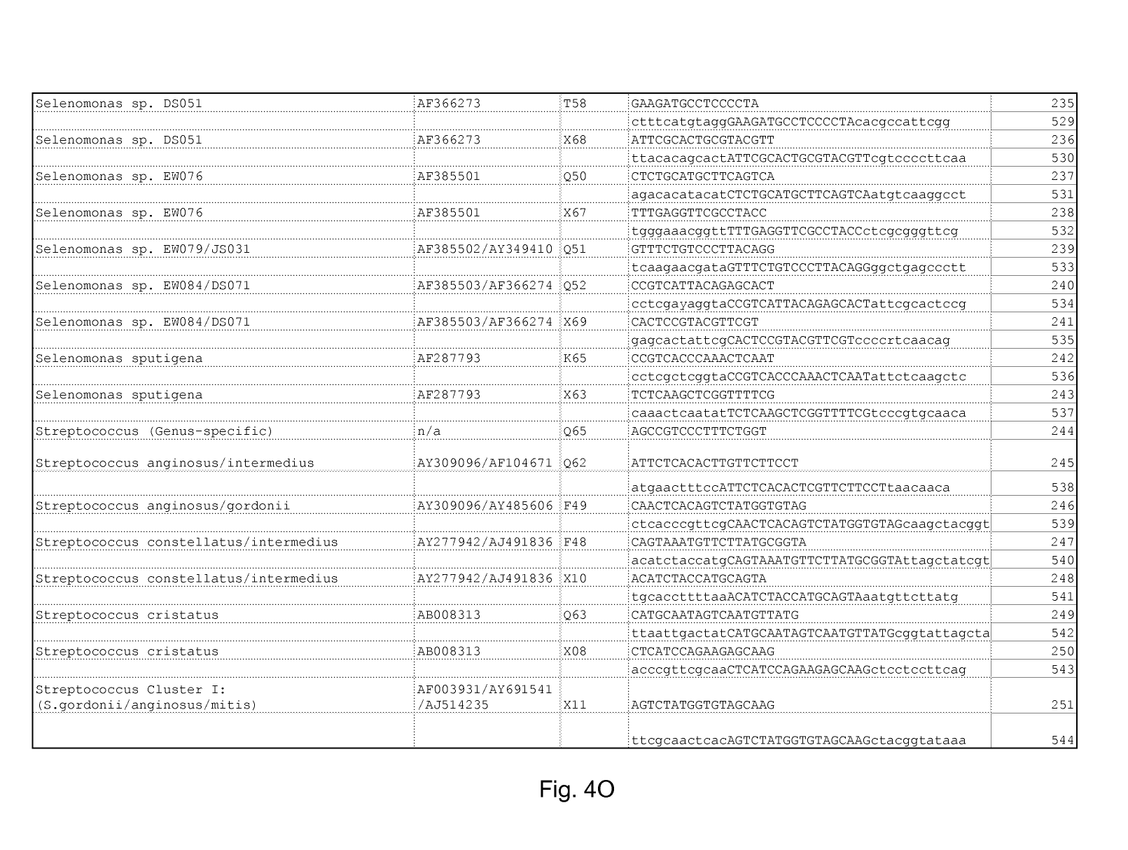

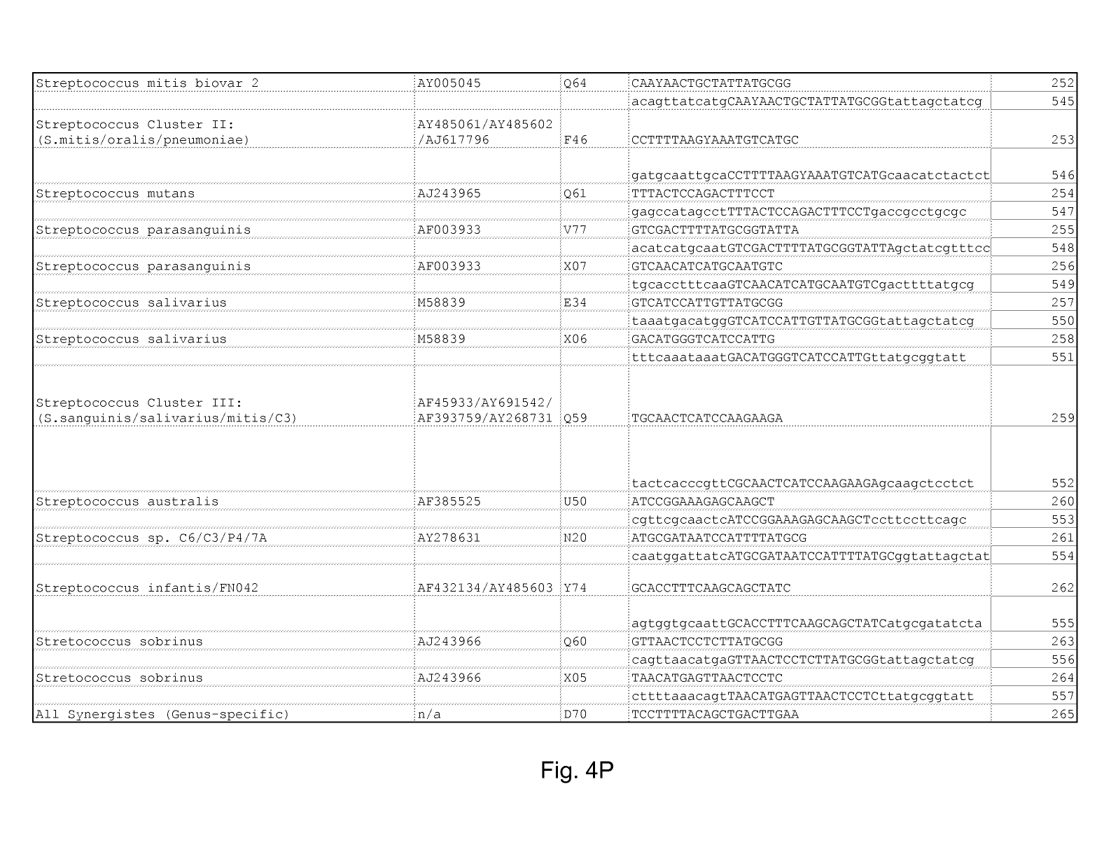

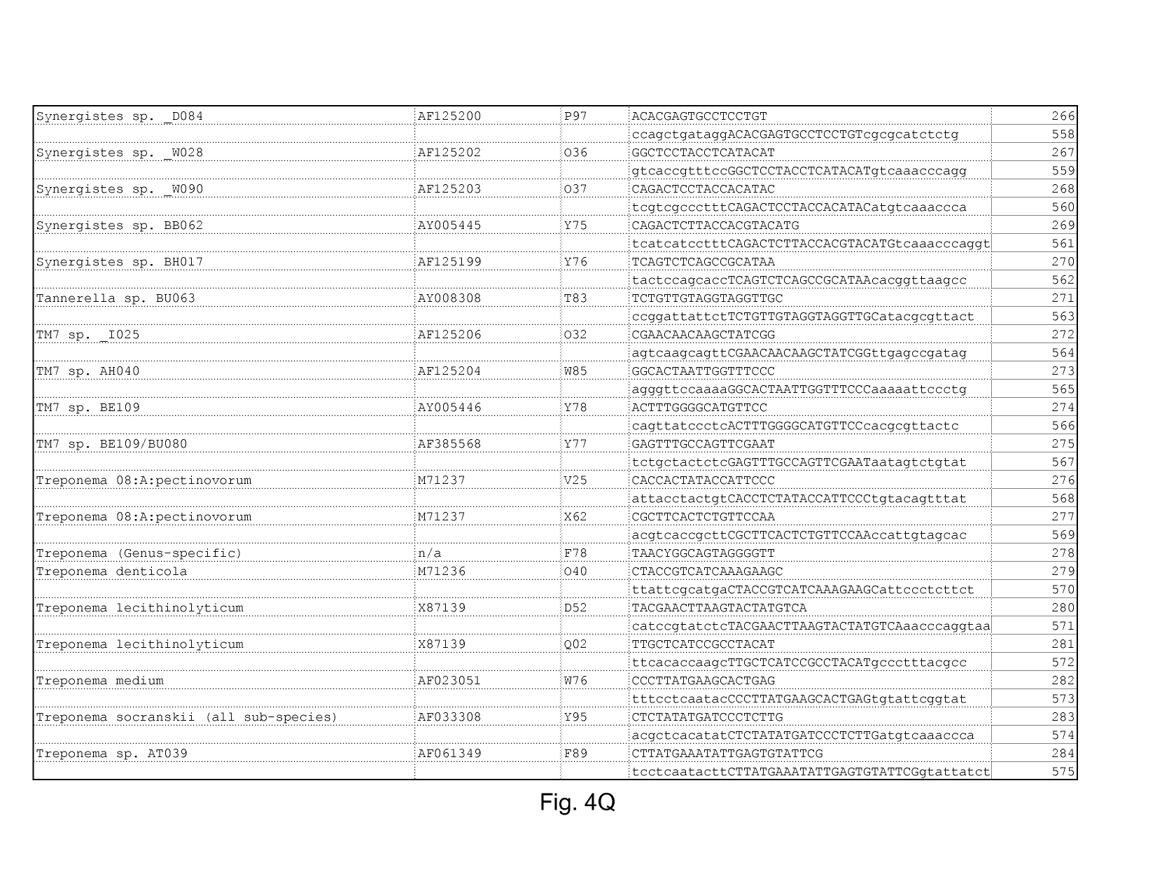

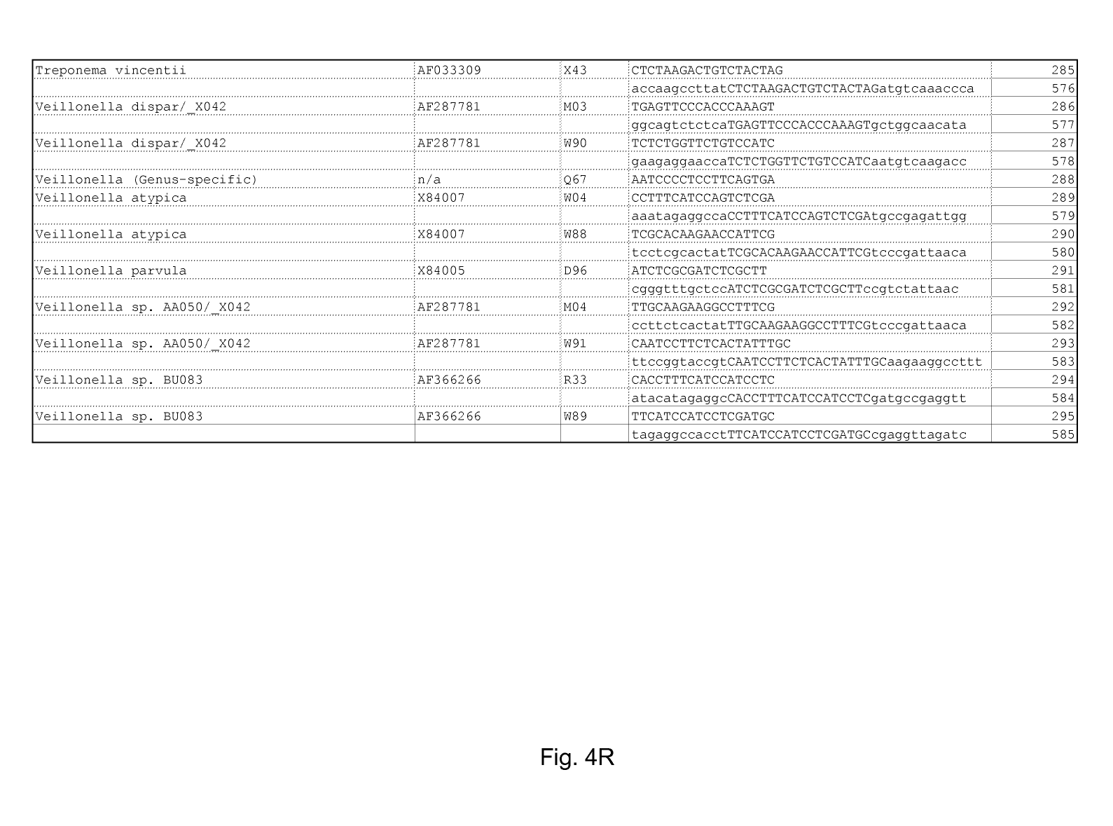

The present invention relates to arrays and methods for identifying a (e.g., one or more) microorganism. The present invention pertains to specific nucleic acid molecules that are useful in identifying organisms, diseases and/or conditions related to human microflora. The nucleic acid sequence of these molecules was determined by studying the divergent regions of the genome of these microorganisms, in particular the 16S rRNA genes, and testing them for their ability to identify the target microorganism. Using these 16S sequences, nucleic acid molecules (e.g., probes) were designed and prepared. (e.g., with a spacer/linker section, as described in more detailed herein) for use in the identification of microorganisms. Specifically, using the protocol described in the Exemplification, about 295 nucleic acid molecules were designed and prepared, and these molecules are used to identify the microorganisms, as shown in FIG. 4, Table 1. In particular, the array identifies bacteria typically found in the oral cavity. Specifically, the array and methods described herein detect one or more microorganisms by detecting nucleic acid molecules in the sample, either bacterial 16S rRNA or 16S rRNA genes. The method or array of the present invention is the first of its kind to have an ability to identify such an extensive number of bacteria of this cavity, and does so in a comprehensive manner so that one can assess the composition of the microflora of the cavity. Arrays for microflora common to other areas (e.g., lungs, blood, skin, vagina, urinary tract, intestinal tract) of the human body are also embodied by the present invention.

The present invention includes methods for assessing the composition of the flora of microorganisms in a sample by assessing the presence or absence of the microorganisms described herein. Specifically, the method includes contacting nucleic acid molecules obtained from a sample with the probes of the present invention. This step occurs under conditions suitable for hybridization to form a complex or hybrid, and the hybrids are detected. The presence of complexes correlate with the microorganisms listed in Table 1, to thereby provide a composition of the microflora of the sample.

Such an analysis is helpful in assessing efficacy or need of oral hygiene products, such as toothpaste, mouthwash, fluoride, breath enhancers, tooth-whitening treatments or floss. For example, one could test the effect of mouthwash on an individual by obtaining samples before and after using the mouthwash and comparing the flora present in each sample (e.g., the number and/or type of bacterial present or absent in the sample). Comparing the compositional flora of each sample allows one to make determinations as to the efficacy of the product. As such, the present invention includes assessing the effect of an oral product on the compositional flora of a sample at one or more time points, and assessing or comparing the presence, absence or both of one or more microorganisms, as described herein.

The methods and arrays of the present invention further embody assessing the efficacy of an oral product independent of the specific microorganism or groups of microorganisms identified. In this embodiment, the probes of the present invention correlate directly to oral health, or to a particular disease or condition, as described further herein. Such a method involves determining the presence, absence, level or percentage of nucleic acid molecules in the sample that hybridize to one or more nucleic acid molecules of the present invention, and comparing or analyzing the presence, absence, level or percentage of the one or more complexes at the one or more time points (e.g., before and after administration of the oral product). Absence is defined herein as the level of a hybrid complex that is below a detectable level or limit. Based on the hybridization that occurs between the probes of the present invention and those found in the sample, a determination of the efficacy of the oral product can be made.

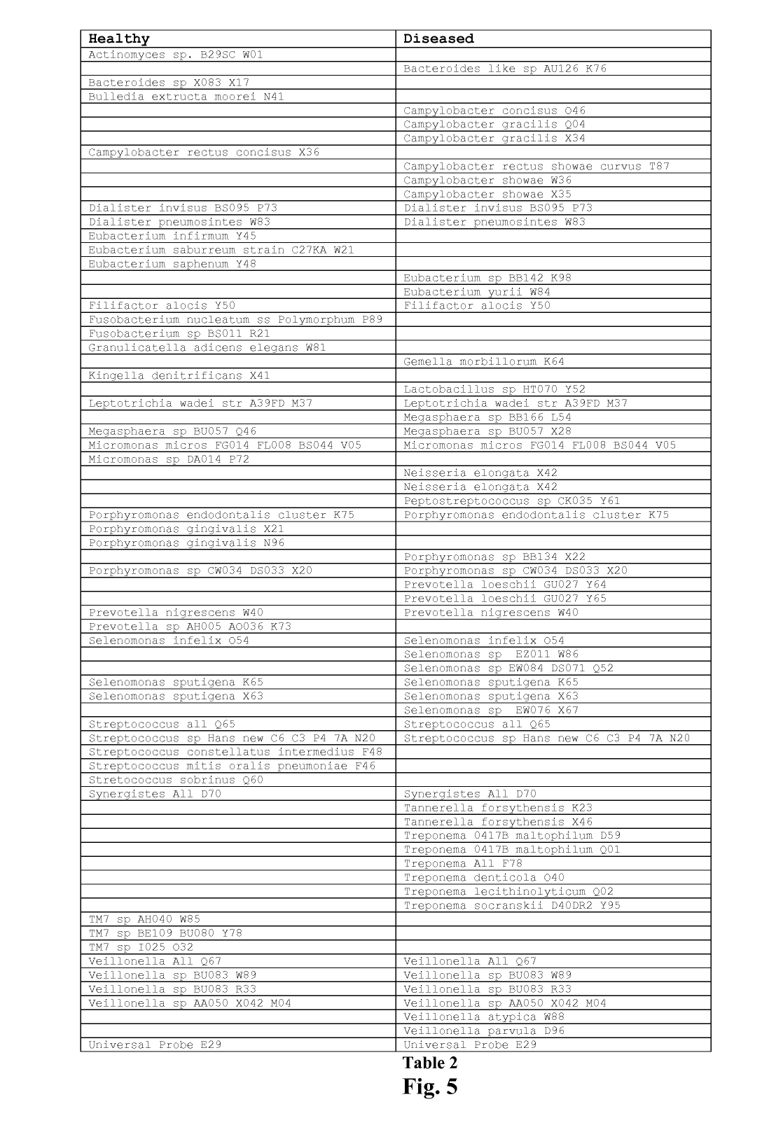

Similarly, the methods of present invention relate to methods of diagnosing patients with a disease or condition, providing a prognosis for a patient, and/or determining the efficacy of treatment. In an embodiment, methods of diagnosing a patient with a disease or condition can be conducted by determining the presence or absence of the microorganism associated with the disease or condition, as described herein. Once the microbe(s) of a particular sample is identified, an individual can be better diagnosed and/or treated for diseases associated with those microbes. For example, FIGS. 3A and B show results from a periodontal patient and a healthy patient. The diseased sample contains a number of disease associated species or phylotypes which are not present in the health sample; namely Bacteroidetes sp. AU126, Campylobacter gracilis, Campylobacter showae, Eubacterium yurii, Eubacterium sp. BB142, Megasphaera sp. BB166, Prevotella loeschii, Tannerella forsythia, Treponema maltophilum, Treponema denticola, Treponema lecithinolyticum, and Treponema socranskii. See FIG. 5, Table 2. In contrast, the healthy sample contains Streptococcus mitis/oralis (e.g., SEQ ID NO: 251, 252, 253, and/or 259) and Streptococcus constellatus/intermedius (e.g., SEQ ID NO: 248 and/or 248), species often associated with health. The results of such a test help a dentist or doctor properly diagnose the disease, and can impact the type of treatment provided to the patient. In yet another embodiment, hybridization of the probes of the present invention can directly correlate with the presence of a disease or condition (e.g., a diagnosis). Such methods include determining the presence or absence of nucleic acid molecules that hybridize to the probes of the present invention, and then determining diseases associated with that pattern (presence and/or absence) of nucleic acid molecules in the sample. Also, in referring to FIGS. 3A and B, nucleic acid molecules in a sample that hybridize with probes having one or more of SEQ ID NO: 30, 31, 34, 35, 46, 47, 52, 53, 86, 87, 91, 92, 133, 134, 177, 178, 278, 279, 280, 281, and/or 283 correlate with periodontal disease, and can be diagnosed in one example on this basis, independent of the specific microorganisms that are present.

Furthermore, the methods of the present invention include monitoring treatment of diseases. For example, the treatment for a periodontal patient above can be monitored after the patient has received the proper treatment with antibiotics, surgery, and/or other dental treatment. As such, one can compare the results of a baseline determination, with one or more determinations made after treatment has begun. In one example, an absence or decrease in the level or percentage (e.g., the level goes from one level to a lower level or even an undetectable level) of certain nucleic acid sequences from the sample that hybridize to nucleic acid sequences of the present invention indicates that treatment is working. Increases in certain levels of hybridization of the nucleic acid molecules of the present invention indicate, in an embodiment, that treatment is not effective. Assessing levels at various stages or time points prior to and/or during the course of treatment provides a physician with information to make better, more informed decisions regarding treatment. As with diagnosis or prognosis of a disease or condition, treatment of the disease or condition can be done on at least two levels: based on the hybridization of the probes of the present invention, or based on the microorganisms or groups thereof that are identified by these probes.

More specifically, the present invention includes, in part, methods for identifying one or more microorganisms through the hybridization of the nucleic acid molecules described herein. Assaying the nucleic acid molecules of the present invention can be conducted using several methods and in one embodiment includes a Southern blot. Briefly, blot techniques include immobilizing or attaching nucleic acid molecules to a solid support, and subjecting or contacting nucleic acid molecules obtained from a sample under conditions for hybridization. Methods for preparing the nucleic acid molecules from the sample are further described herein. In nucleic acid hybridization reactions, the conditions used to achieve a particular level of stringency are described herein and depend on the nature of the nucleic acids being hybridized. For example, the length (e.g., 18-24 mer), degree of complementarity, nucleotide sequence composition (e.g., GC v. AT content), and nucleic acid type (e.g., RNA v. DNA v. PNA) of the hybridizing regions of the nucleic acids can be considered in selecting hybridization conditions.

In addition, amplification of polynucleotide sequence by, for example, the polymerase chain reaction (PCR) technique, further described herein, can serve the same purpose. By properly choosing the primers, one can obtain an amplified product of an expected size after a certain plurality of PCR cycles if the target sequence is present in the extracted sample containing nucleic acids or genetic material. This method offers sensitivity, since a 30-cycle reaction can generate an amplification on the order of 10⁹.

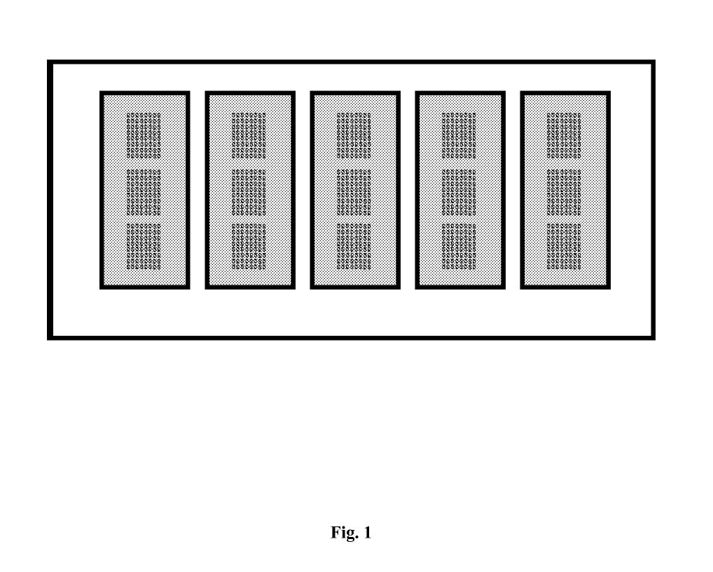

In a preferred embodiment, methods for identifying a nucleic acid sequence involve the use of an array. An “array,” “microarray,” “DNA chip,” “biochip,” or “oligo chip” may be used interchangeably and refers to a grid of spots or droplets of genetic material of known sequences in defined locations or known positions. The advantage of using an array is the ability to test a sample against hundreds of nucleic acid sequences at once. The array of probes can be laid down in rows and columns. As shown in FIG. 1, five arrays (64×64 droplets) are arranged on a glass support, and the same array is repeated three times. The actual physical arrangement of probes on the chip is not essential, provided that the spatial location of each probe in an array is known. When the spatial location of each probe is known, the data from the probes can be collected and processed. In processing the data, the hybridization signals from the respective probes can be reasserted into any conceptual array desired for subsequent data reduction whatever the physical arrangement of probes on the chip. The present invention includes arrays having one or more of the nucleic acid molecules described herein (any one of SEQ ID NOs:1-295; a nucleic acid sequence having between about 80% and about 100% of contiguous nucleotides of any one of SEQ ID NO: 1-295; any one of SEQ ID NOs:296-585; a nucleic acid sequence having between about 15 and about 25 contiguous nucleotides of any one of SEQ ID NO: 296-585; a reverse complement thereof, and any combination thereof) bound thereto.

The present invention encompasses combinations of the nucleic acid molecules described herein arranged in an array. The array can be tailored to identify certain classes of microorganisms, certain genera, or particular clinical diseases or conditions (e.g., a periodontitis array). As such, the present invention includes having a percentage of the nucleic acid molecules having a sequence of SEQ ID NOs:1-585 formatted into an array. For example, the present invention includes an array having at least about 100%, 90%, 80%, 70%, 60%, 50%, 40%, 30% 20% or 10% of the nucleic acid molecules disclosed herein. The present invention also includes having a particular combination of the nucleic acid molecules described herein (e.g., about 550, 500, 450, 400, 350, 300, 250, 200, 180, 160, 140, 120, 100, 80, 60, 40, 20, 10 of the nucleic acid molecules described herein) arranged in an array format.

The genetic material is systematically arranged on a solid support that includes, e.g., glass, silica chips, nylon (polyamide) membrane, polymer, plastic, ceramic, metal, coated on optical fibers, or infused into gel, matrix. Examples of the type of solid support can be a slide, plate, chip, dipstick, or other types known in the art or later developed. The solid support can also be coated to facilitate attachment of the oligonucleotides to the surface of the solid support. Any of a variety of methods known in the art may be used to immobilize oligonucleotides to a solid support. The oligonucleotides can be attached directly to the solid supports by epoxide/amine coupling chemistry. See Eggers et al. Advances in DNA Sequencing Technology, SPIE conference proceedings (1993). Another commonly used method consists of the non-covalent coating of the solid support with avidin or streptavidin and the immobilization of biotinylated oligonucleotide probes. By oligonucleotide probes is meant nucleic acid sequences complementary to a species-specific target sequence. In one embodiment, probes are attached to a glass solid support through aldehyde/amine coupling chemistry, as described in the Exemplification.

Using a solid support having the nucleic acid molecules bound thereto, the method of the present invention involves contacting the nucleic acid molecules described herein with nucleic acid molecules obtained from a sample to be tested under conditions suitable for hybridization with one another. A sample is obtained from the individual to be tested and can consist of saliva, plaque, sputum, aspirate, blood, plasma, cerebrospinal fluid, aspirate, tissue, skin, urine, mucus, or cultured organisms grown in vitro. The sample obtained can be related to the type of array that is being utilized. For example, in the case of an array for the oral cavity, a plaque sample is preferable and can be obtained by scraping the plaque with a sterile instrument. The DNA of the sample can be amplified and labeled so that it is suitable for hybridizing with the nucleic acid molecules of the present invention. The term, “amplifying,” refers to increasing the number of copies of a specific polynucleotide. For example, PCR is a method for amplifying a polynucleotide sequence using a polymerase and two oligonucleotide primers, one complementary to one of two polynucleotide strands at one end of the sequence to be amplified and the other complementary to the other of two polynucleotide strands at the other end. Because the newly synthesized DNA strands can subsequently serve as additional templates for the same primer sequences, successive rounds of primer annealing, strand elongation, and dissociation produce rapid and highly specific amplification of the desired sequence. PCR also can be used to detect the existence of the defined sequence in a DNA sample. The DNA of the sample is amplified or replicated, in one embodiment, with PCR. Methods of PCR are known in the art and are described for example in Mullis, K. B. Scientific American 256:56-65 (1990).

Briefly, PCR is performed with the use of a DNA polymerase enzyme and include, for example, one that is isolated from a genetically engineered bacterium, Thermus aquaticus (Taq). Other DNA polymerases include, e.g., ThermalAce™ high fidelity polymerase (Invitrogen), TthI polymerase, VENT polymerase or Pfu polymerase. The polymerase, along with the primers and a supply of the four nucleotide bases (adenine, guanine, cytosine and thymine) is provided. Under certain conditions (e.g., 95° C. for 30 seconds), the DNA is denatured to allow the strands to separate. As the DNA solution cools, the primers bind to the DNA strands, and then the solution is heated to promote the Taq polymerase to take effect. Mullis, K. B. Scientific American 256:56-65 (1990). Other known methods, or methods developed in the future can be used so long as the DNA of the sample is amplified or replicated.

In an embodiment, a single round of PCR is performed, and then the sample is labeled in a second round of PCR, as described herein. Several labels exist to facilitate detection of a nucleic acid molecule complex. Techniques for labeling and labels, that are known in the art or developed in the future, can be used. In a preferred embodiment, the label is simultaneously incorporated during the amplification step in the preparation of the sample nucleic acids. For example, PCR with labeled primers or labeled nucleotides will provide a labeled amplification product. The nucleic acid (e.g., DNA) is amplified in the presence of labeled deoxynucleotide triphosphates (dNTPs). In a preferred embodiment, transcription amplification, as described above, using a labeled nucleotide (e.g., fluorescein-labeled UTP and/or CTP) incorporates a label into the transcribed nucleic acids.

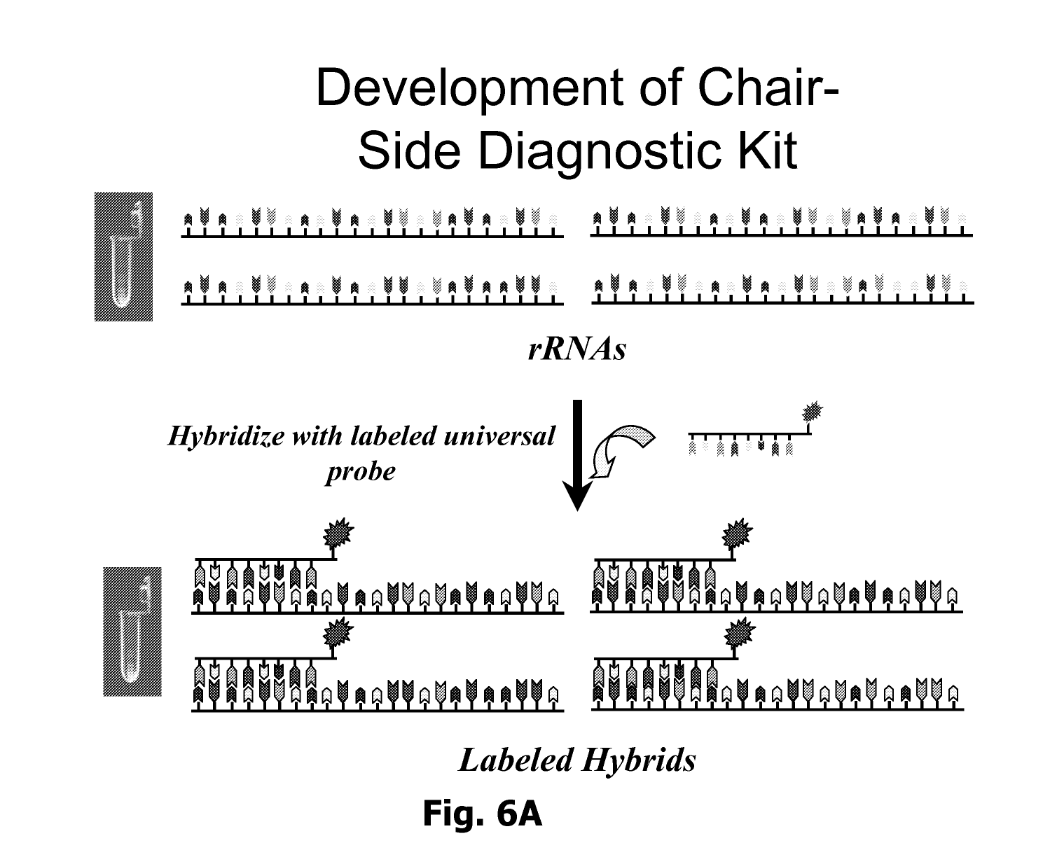

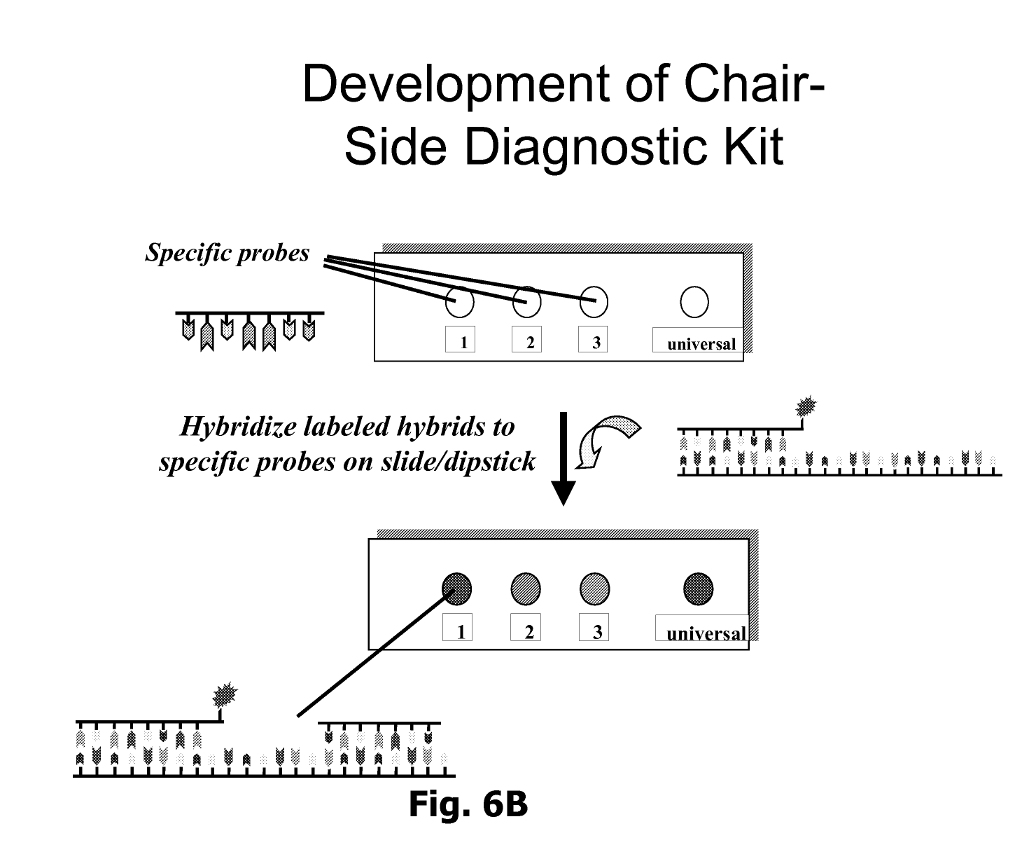

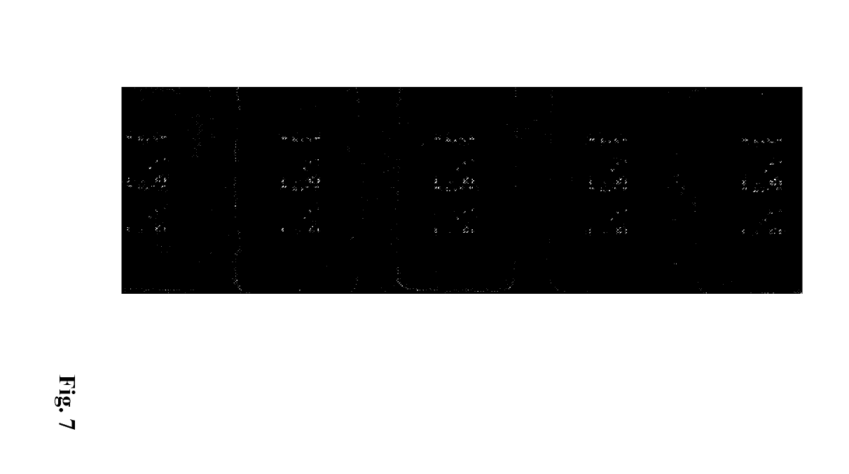

The present invention can be performed with and without PCR amplification. In the embodiment of conducting the methods without PCR amplification, the nucleic acid (e.g., rRNA) can be obtained from a sample and labeled by, e.g., universally labeled probes that hybridize to a portion of the rRNA. The steps of these methods are shown in FIGS. 6A and 6B for a chair-side diagnostic kit. The labeled rRNA is subjected to or contacted with the nucleic acid molecules of the present invention under conditions suitable for hybridization, as further described herein. In this embodiment, the nucleic acid molecules of the present invention hybridize to a portion of the rRNA that is different than the portion to which the universal probe hybridizes. Hence, a complex forms between the rRNA from the sample, the labeled universal probe and the nucleic acid of the present invention (e.g., the probes that identify microorganisms). The complex is then detected as described herein. The types of solid supports are also described herein, but in this embodiment a slide or dipstick is preferable. Additionally, rRNA from the sample can be obtained using methods known in the art and reverse transcribed using a reverse transcriptase (or relaxed polymerase) to make a DNA copy. The DNA can then be amplified and labeled using PCR, as described herein.

Alternatively, a label may be added directly to the original nucleic acid sample (e.g., rRNA or rDNA) or to the amplification product after the amplification is completed. Such labeling can result in the increased yield of amplification products and reduce the time required for the amplification reaction. Means of attaching labels to nucleic acids include, for example nick translation or end-labeling (e.g., with a labeled RNA) by kinasing of the nucleic acid and subsequent attachment (ligation) of a nucleic acid linker joining the sample nucleic acid to a label (e.g., a fluorophore).

Detectable labels suitable for use in the present invention include any composition detectable by spectroscopic, photochemical, biochemical, immunochemical, electrical, optical or chemical means. The most frequently used labels are fluorochromes like Cy3, Cy5 and Cy7 suitable for analyzing an array by using commercially available array scanners (e.g., Axon, General Scanning, and Genetic Microsystem). Other labels that can be used in the present invention include biotin for staining with labeled streptavidin conjugate, magnetic beads (e.g., Dynabeads™), dendrimers, fluorescent proteins and dyes (e.g., fluorescein, texas red, rhodamine, green fluorescent protein, and the like, see, e.g., Molecular Probes, Eugene, Oreg., USA), radioactive labels (e.g., ³H, ¹²⁵I, ³⁵S, ¹⁴C, or ³²P), enzymes (e.g., horse radish peroxidase, alkaline phosphatase and others commonly used in an ELISA), and calorimetric labels such as colloidal gold (e.g., gold particles in the 40-80 nm diameter size range scatter green light with high efficiency) or colored glass or plastic (e.g., polystyrene, polypropylene, latex, etc.) beads. Patents teaching the use of such labels include WO 97/27317, and U.S. Pat. Nos. 3,817,837; 3,850,752; 3,939,350; 3,996,345; 4,277,437; 4,275,149; and 4,366,241.

A fluorescent label is preferred because it provides a very strong signal with low background. It is also optically detectable at high resolution and sensitivity through a quick scanning procedure. The nucleic acid samples can all be labeled with a single label, e.g., a single fluorescent label. Alternatively, in another embodiment, different nucleic acid samples can be simultaneously hybridized where each nucleic acid sample has a different label. For instance, one target could have a green fluorescent label and a second target could have a red fluorescent label. The scanning step will distinguish cites of binding of the red label from those binding the green fluorescent label. Each nucleic acid sample (target nucleic acid) can be analyzed independently from one another.

The sample can be purified to remove unincorporated label or dye. Once the sample is prepared, it can be subjected to the nucleic acid molecules of the present invention for hybridization. Hybridization refers to the association of single strands of polynucleotides through their specific base-pairing properties to form a complementary double-stranded molecule. With respect to the present invention, the labeled DNA of the sample hybridizes with the oligonucleotides on the solid support. Hybridization conditions include variables such as temperature, time, humidity, buffers and reagents added, salt concentration and washing reagents. Preferably, hybridization occurs at high stringency conditions (e.g., 55° C., for 16 hours, 3×SSC). Examples of stringency conditions are described herein. Methods for hybridization are known, and such methods are provided in U.S. Pat. No. 5,837,490, by Jacobs et al. The solid support can then be washed one or more times with buffers to remove unhybridized nucleic acid molecules. Hybridization forms a complex between the nucleic acid of the present invention and nucleic acid of the sample.

Hybridization assay procedures and conditions will vary depending on the application and are selected in accordance with the general binding methods known including those referred to in: Maniatis et al. Molecular Cloning: A Laboratory Manual (2.sup.nd Ed. Cold Spring Harbor, N.Y., 1989); Berger and Kimmel Methods in Enzymology, Vol. 152, Guide to Molecular Cloning Techniques (Academic Press, Inc., San Diego, Calif., 1987); Young and Davism, P.N.A.S, 80: 1194 (1983). Methods and apparatus for carrying out repeated and controlled hybridization reactions have been described in U.S. Pat. Nos. 5,871,928, 5,874,219, 6,045,996 and 6,386,749, 6,391,623.

The complex, which is labeled, can be detected and quantified. Detection of the array can be performed by autoradiography or in real time to determine the presence of hybridized products at particular locations on the solid support. In particular, detection can occur using scanners that emit light from a laser at specific frequency. In one example, an Axon GenePix 4000B microarray scanner set at 532 nM wavelength was used. Scanners and other devices, including those known and later developed, for detecting the labeled hybridized complexes can be used. These measurements are converted to electronic signals that can be analyzed. The raw data optionally are filtered and/or normalized. Filtering refers to removing data from the analysis that does not contribute information to the experimental outcome, e.g., does not contribute to the identification of a microbe. Normalizing data refers to, in one embodiment, a linear transformation to correct for variables within the experimental process.

In addition to detecting the presence or absence (e.g., below a detectable threshold), quantification can also occur and be provided in a level or percentage. While in one embodiment, as shown in the Exemplification, presence or absence of hybridization is demonstrated, signal intensity relative to other probes can also be used for quantification. As a general rule, the more hybridization of complexes that is present (e.g., presence of the microorganism in the sample), the more intense the probe signal. In an embodiment in which PCR amplification occurs, the intensity does not directly reflect absolute numbers, but rather is proportional to a relative amount in the original sample. Such quantification of hybridization complexes can be carried out using methods known in the art.

The data can then be analyzed by a qualified person or computerized system. In an embodiment, the presence of hybridization of the nucleic acid molecules of the present invention correlates to the presence of the corresponding microbe in the sample. One can compare the spot having a detectable hybrid complex, against a table or database containing information about the spots on which the nucleic acid molecules were bound, and with which particular microorganism they correlate. FIG. 4 has a table that lists the microorganisms and the sequence of the probe to which they correlate. After such a comparison, the microorganism can be identified in the sample. One or more nucleic acid molecules can correlate to a particular microorganism, closely related microorganisms, or genus. In some embodiments, at least 2 probes correlate to or identify a microorganism, as defined herein. Having more than one occurrence of hybridization with more than one probe can, in some embodiments, provide for a more accurate identification.

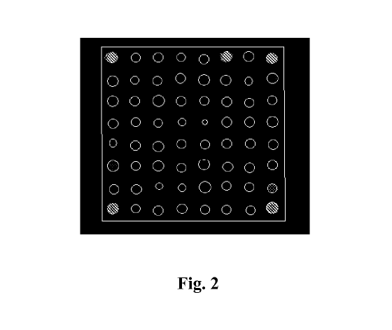

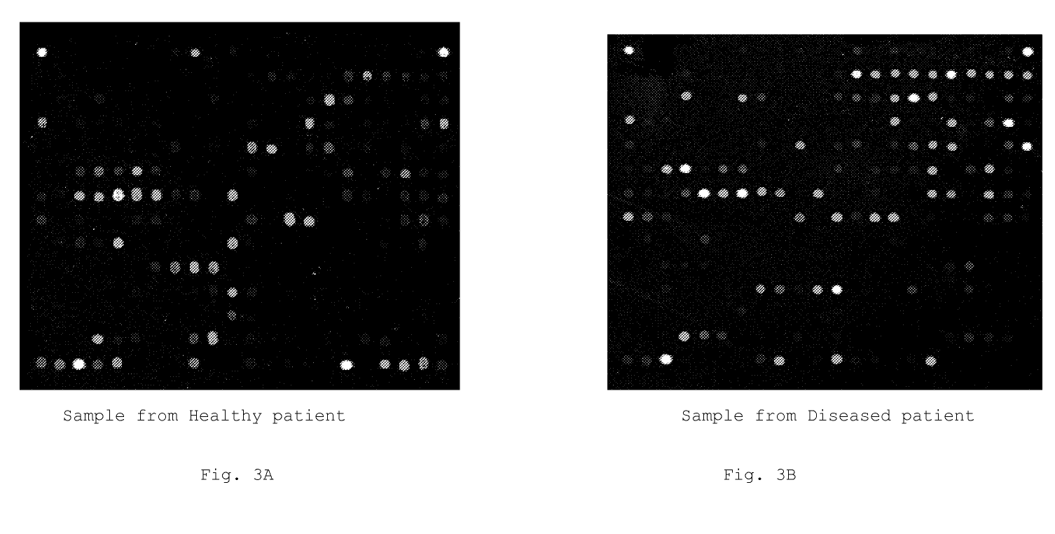

The presence of hybridization, as detected in some embodiments by fluorescence, is compared to controls (e.g., positive and/or negative controls). For example, in one embodiment, and as shown in the Exemplification and in the 4 corners of the array shown in FIGS. 2 and 3, positive controls were used. A universal probe, shown in Table 1, was placed in each corner of the array. The universal probe identifies a section of 16S rRNA that is common in all microorganisms that are being tested in the Exemplification. Such a control not only shows that the hybridization is occurring, but it is occurring in various areas throughout the array. Negative controls can also be used. Negative controls, in an embodiment, include a 16S rRNA capture probe for an organism only found in non-human environments such as acid mine drainage, or hyperthermal ponds. Such controls assist in determining the existence of any background noise (e.g., fluorescence). Array technology, as described herein, allows for the identification of a number (e.g., at least about 50, about 100, about 150, about 200) of microorganisms at one time.

The methods of the present invention also involve determining the level or percentage of a particular microbe in a sample. Data can be generated for mean detection levels or percentage of known quantities of a microorganism, and can be used to compare a sample of unknown quantity to determine the level or percentage of the microorganism in the sample. In one embodiment, threshold levels or percentages (e.g., low, medium and high) of bacteria can be established using known quantities of bacteria, and compared to an unknown level or percentages of bacteria in a sample. Detection of one or more bacteria above the high threshold level signifies high quantities of the particular bacteria, detection of a medium threshold level indicates a mid-level quantity of the bacteria in the sample, and detection of bacterial below the low threshold levels indicate low quantities of the bacteria in the sample.

The terms “microorganism” and “microbe” are used in its broadest sense and include those that are known and named, and those that have not yet been named or cultivated. The term “microorganism” includes single species, phylotypes, closely related species or phylotypes, genus, and higher taxon. As a general rule, bacterial strains of species or phylotypes have less than about a 2% difference in 16S rRNA. Closely related species or phylotypes generally have between about a 2% and about a 4% difference in 16S rRNA, whereas a genus often has between about a 5% and about a 10% difference in 16S rRNA. These are simply general guidelines. The probes identify specific species/phylotypes of microorganisms, closely related species and in some cases a particular genus. As used herein, the phrase “identifying a microorganism” refers to the determination of the genus, closely related microorganisms, as well as the species/phylotype of a microorganism, including those that are known, unnamed or uncultivated (e.g., those known from strains or 16S rRNA clones). Hence, in an embodiment of the invention, the microarray contains four types of probes. The first type is specific for a species or phylotype. The second is specific for two or more species or phylotypes of neighboring taxa. The third type of probe is designed to recognize species in a genera or higher taxonomic level. The fourth type of probe is our bacterial universal probe which recognizes all bacteria recognized by the other three classes of probes. Thus, a single organism can be recognized by multiple, hierarchal probes. However, an embodiment of the invention includes a microarray is useful in diagnosing or assessing diseases or conditions, as described herein, as direct indicators of that disease or condition, independent of the particular microorganism that may be associated with it.

Examples of microorganisms are found in FIG. 4, Table 1, and include Gram negative aerobic bacteria, Gram positive aerobic bacteria, Gram negative microaerophilic bacteria, Gram positive microaerophilic bacteria, Gram negative facultatively anaerobic bacteria, Gram positive facultatively anaerobic bacteria, Gram negative anaerobic bacteria, Gram positive anaerobic bacteria, Gram positive asporogenic bacteria, Actinomycets. Uncultivated or unnamed microorganisms can also be identified by the methods described herein. Uncultivated microorganisms are described by its similarity of the nucleic acid molecules used in the assay of the present invention to the sequence of the microorganism's 16S rDNA in a public sequence database, such as GenBank. Additionally, “microorganism” refers to live, dead, fragmented or lysed organisms.

The present invention includes methods of making an array. The method includes selecting a solid support, as described herein. In one embodiment, aldehyde glass slides were used. The nucleic acid molecules shown in FIG. 4 can be synthesized by standard methods, and spotted onto the solid support, or they can be synthesized directly on the chip (in situ or in silico) through known processes. In one aspect, the nucleic acid molecules of the present invention can be grown on the solid support or integrated on the solid support using flow channels. Methods of forming high density arrays of oligonucleotides that are now known or developed in the future can be used to construct the array of the present invention, namely an array having the nucleic acid molecules described herein. In particular, arrays can be synthesized on a solid substrate by a variety of methods, including, but not limited to, light-directed chemical coupling, and mechanically directed coupling. See Pirrung et al., U.S. Pat. No. 5,143,854 (see also PCT Application No. WO 90/15070) and Fodor et al., PCT Publication Nos. WO 92/10092 and WO 93/09668 which disclose methods of forming vast arrays. See also, Fodor et al., Science,251, 767-77 (1991). One example of synthesizing a polymer array includes the VLSIPSTM approach. Additionally, methods which can be used to generate an array of oligonucleotides on a single substrate can be used. For example, reagents are delivered to the substrate by either (1) flowing within a channel defined on predefined regions or (2) “spotting” on predefined regions. However, other approaches, as well as combinations of spotting and flowing, or other approaches can be employed.

The method further includes preparing the nucleic acid molecules for attachment to the solid support. Optionally, a spacer that provides a space between the support and the capture nucleotide sequences can be used to increase sensitivity of the array. A spacer that can be used with the present invention includes any molecular group that allows the nucleic acid molecule to remain off of or separated from the support. Another example of a spacer is a hexaethylene glycol derivative for the binding of small oligonucleotides upon a membrane. Patent publication No.: EP-0511559. In one embodiment of the invention, the nucleic acid probes of this invention comprise at least two parts, the specific probe, and the spacer/linker section. The specific probe portion comprises about 14-30 nucleic acids or nucleic acid mimetics (e.g., PNAs). The spacer/linker is comprised of anything that positions the specific probe away from the substrate and that adheres or attaches the specific probe to the substrate. Alternatively, probes can be attached to a gel, in which case, a spacer/liner is not necessary.

The nucleic acid molecules of the present invention can also be prepared to promote attachment to the solid support chosen, or to react with a coating placed on the support. The solid support can be coated to promote adherence to the support, and once the nucleic acid molecule is applied, in some cases ultraviolet irradiation allows for DNA fixation. For example, the nucleic acid molecules of the present invention or the solid support can be modified to react with substrates including amine groups, aldehydes or epoxies to promote attachment. As shown in the Exemplification, the 18-24-mer oligonucleotides were synthesized with eight spacer thymidines and a linear C6 aliphatic amine modification on the 5′ end. The nucleic acid molecules of the present invention were then attached to glass through Schiff base formation, which occurs naturally at room temperature. The Schiff base is then reduced with sodium borohydride to form a stable covalent bond. The probes of the present invention can be further prepared by diluting solution containing the probes to the desired concentration, as shown in the Exemplification. Methods, now known or developed later, for promoting attachment of the nucleic acid to the solid support can be used.

The nucleic acid molecules of the present invention can be applied to the solid support with a spotter, a robotic machine that applies the droplets of the nucleic acid molecules of the present invention to a well or spot on the array. Many spotters used ink jet technology or the piezoelectric capillary effect to complete the grid of probe droplets. Spotting the nucleic acid molecules onto the solid support is often referred to as “printing.” The droplets of the nucleic acid molecules can be arranged in a desired format, so long as each sequence is bound to the surface in a different localized area. Multiple arrays can be placed on a single support, and the same array can be repeated more than once. In one example, a single array was printed three times in five separate locations on a slide. This allows five separate clinical samples to be analyzed in triplicate.

The present invention includes kits. Kits can include the array of the present invention, as described herein. Kits can also include reagents that are used to carry out hybridization. Examples of such regents include labeling reagents, primers (labeled and/or unlabeled), buffers and washing solutions. Labeling reagents include labels, as described herein (e.g., fluorescent dyes, streptavidin conjugate, magnetic beads, dendrimers, radiolabels, enzymes, calorimetric labels, nanoparticles, and/or nanocrystals) including Cy3 and Cy5. The kit can also include software use to analyze the results, as described herein.

The present invention, in one embodiment, includes an isolated nucleic acid molecule having a nucleic acid sequence of any one of SEQ ID NOs:1-295; a nucleic acid sequence having between about 80% and about 100% of contiguous nucleotides of any one of SEQ ID NO: 1-295; any one of SEQ ID NOs:296-585; a nucleic acid sequence having between about 15 and about 25 contiguous nucleotides of any one of SEQ ID NO: 296-585; a sequences that hybridizes thereto; a reverse complement thereof, and any combination thereof. The present invention includes sequences as recited in FIG. 4.

As used herein, the terms “DNA molecule” or “nucleic acid molecule” include both sense and anti-sense strands, cDNA, complementary DNA, recombinant DNA, RNA, wholly or partially synthesized nucleic acid molecules, PNA and other synthetic DNA homologs. A nucleotide “variant” is a sequence that differs from the recited nucleotide sequence in having one or more nucleotide deletions, substitutions or additions so long as the molecules binds to the nucleic acid molecules of the present invention including its reverse complement. Such variant nucleotide sequences will generally hybridize to the recited nucleotide sequence under stringent conditions.

As used herein, an “isolated” gene or nucleotide sequence which is not flanked by nucleotide sequences which normally (e.g., in nature) flank the gene or nucleotide sequence (e.g., as in genomic sequences). Thus, an isolated gene or nucleotide sequence can include a nucleotide sequence which is designed, synthesized chemically or by recombinant means.

Also encompassed by the present invention are nucleic acid sequences, DNA or RNA, PNA or other DNA analogues, which are substantially complementary to the DNA sequences and which specifically hybridize with their DNA sequences under conditions of stringency known to those of skill in the art. As defined herein, substantially complementary means that the nucleic acid need not reflect the exact sequence of the sequences of the present invention, but must be sufficiently similar in sequence to permit hybridization with nucleic acid sequence of the present invention under high stringency conditions. For example, non-complementary bases can be interspersed in a nucleotide sequence, or the sequences can be longer or shorter than the nucleic acid sequence of the present invention, provided that the sequence has a sufficient number of bases complementary to the DNA of the microorganism to be identified to allow hybridization therewith.

In another embodiment, the present invention includes molecules that contain at least about 15 to about 25 contiguous nucleotides or longer in length (e.g., 16, 17, 18, 19, 20, 21, 22, 23 or 24) of any nucleic acid molecules described herein, and preferably of SEQ ID NO: 1-295. Alternatively, molecules of the present invention includes nucleic acid sequences having contiguous nucleotides of about 80% and about 100% of the length of any one of the sequences described herein, and preferably of SEQ ID NO: 1-295. The targets (e.g., SEQ ID NO: 295-595) provided herein can be used, but modified slightly by shifting the target in the bacterial rRNA sequence by about 1 to about 12 nucleic acid bases in either direction (3′ or 5′). In such a case, an overlap the target sequence described herein occurs. Shifting the probe's target nucleic acid molecules by a few bases would allow one, in some cases, to still identify the particular microorganism. When shifting of about 1 to about 12 bases of the 16-24 mer polynucleotide occurs, at least about 6 contiguous nucleotides of the sequences shown in FIG. 4 are used. When shifting of about 3 to about 6 bases of the 16-24 mer polynucleotide occurs, at least about 15 contiguous nucleotides of the sequences shown in FIG. 4 are used. Along the same lines, the nucleic acid molecules of the present invention can contain about 6 bases of the probes and up to about 24 bases of adjacent sequence from the 16S rDNA as provided in Table 1. Consequently, the nucleic acid molecules of the present invention can have about 30% or greater (about 40%, 50%, 60%, 70%, 75%, 80%, 85%, 90% or 95%) of contiguous nucleotides of the nucleic acid sequence described herein.

Similarly, the present invention includes nucleic acid probes that comprise the nucleic acid sequence of SEQ ID NO: 1-585 and/or is of sufficient length and complementarity to specifically hybridize to a nucleic acid sequence that identifies the corresponding microorganism. The requirements of sufficient length and complementarity can be determined by one of skill in the art. Suitable hybridization conditions (e.g., high stringency conditions) are also described herein.

Specific hybridization can be detected under high stringency conditions. “Stringency conditions” for hybridization is a term of art which refers to the conditions of temperature and buffer concentration which permit and maintain hybridization of a particular nucleic acid to a second nucleic acid; the first nucleic acid may be perfectly complementary to the second, or the first and second may share some degree of complementarity which is less than perfect. For example, certain high stringency conditions can be used which distinguish perfectly complementary nucleic acids from those of less complementarity. “High stringency conditions” for nucleic acid hybridizations and subsequent washes are explained, e.g., on pages 2.10.1-2.10.16 and pages 6.3.1-6 in Current Protocols in Molecular Biology (Ausubel, et al., In: Current Protocols in Molecular Biology, John Wiley & Sons, (1998)). The exact conditions which determine the stringency of hybridization depend not only on ionic strength, temperature and the concentration of destabilizing agents such as formamide, but also on factors such as the length of the nucleic acid sequence, base composition, percent mismatch between hybridizing sequences and the frequency of occurrence of subsets of that sequence within other non-identical sequences. Thus, high stringency conditions can be determined empirically.

By varying hybridization conditions from a level of stringency at which no hybridization occurs to a level at which hybridization is first observed, conditions which will allow a given sequence to hybridize (e.g., selectively) with the most similar sequences in the sample can be determined. Exemplary conditions are described in the art (Krause, M. H., et al., 1991, Methods Enzymol. 200:546-556). Also, low and moderate stringency conditions for washes are described (Ausubel, et al., In: Current Protocols in Molecular Biology, John Wiley & Sons, (1998)). Washing is the step in which conditions are usually set so as to determine a minimum level of complementarity of the hybrids. Generally, starting from the lowest temperature at which only homologous hybridization occurs, each ° C. by which the final wash temperature is reduced (holding SSC concentration constant) allows an increase by 1% in the maximum extent of mismatching among the sequences that hybridize. Generally, doubling the concentration of SSC results in an increase in Tm of about 17° C. Using these guidelines, the washing temperature can be determined empirically for high stringency, depending on the level of the mismatch sought. In some embodiments, high stringency conditions include those in which nucleic acid with less than a few mismatches does not bind. Specific high stringency conditions used to carrying out the steps of the present invention are described in the Exemplification. High stringency conditions, using these guidelines, lie in a temperature range between about 40° C. and about 60° C., an SSC concentration range between about 1× and about 10× (e.g., about 2×), and a reaction time range of between about 30 seconds and about 36 hours.

### EXEMPLIFICATION

### Example 1

### Human Oral Microbial Identification Microarray

Methods

Ordering of Oligonucleotides for Printing.

18-24mer oligos are synthesized by Sigma with a linear C6 aliphatic amine modification on the 5′ end. This modification allows for attachment to Aldehyde-coated slides by means of a Schiff Base formation. An additional 8T spacer is synthesized as well, to help keep distance between the bound oligonucleotides and the glass substrate for more efficient hybridization.

5′-(C6)AmineModification-TTTTTTTT-oligonucleotide
sequence-3′

Printing of Arrays.

Schott-Nexterion Slides AL (formerly Quantifoil QMT Aldehyde Slides) are used. 200 uM Oligos are plated in v-bottom 384 well plates in a 2:1 solution with Nexterion 2× spotting buffer giving a final spot concentration of 100 uM.

Printing is performed using the GeneMachines OmniGrid Arrayer, at 55% humidity and the array layout is set up using the compatible software. Once the slides have been printed, they are labeled by etching with a diamond pencil, and stored in a labeled slide box.

www.quantifoil.com

http://www.genemachines.com/omnigrid/omnigrid.html

Immobilization.

This step is to be performed immediately following the printing. Slides are incubated for 15 min. in a humid chamber consisting of ddH20 at room temperature, and then baked array side up for 1 hour at 120° C.

Once completed, the slides can be stored in the slide boxes, in a dessicator until ready for use.

Blocking.

Immediately before use, the slides must be blocked to reduce non-reactive primary alcohols and get ride of unreacted Aldehyde groups—to minimize fluorescent background after hybridization. This is done using a solution of sodium borohydride (NaBH₄), 1×PBS and 99% EtOH.

First, the slides are extensively washed to remove un-bound DNA molecules and buffer substances by a series of washing steps carried out in glass Wheaton tanks which are filled with rinsing solutions (10% SDS and H20 or just H20). Then they are placed in a tank filled with the NaBH₄ solution for 15 minutes at room temperature.

Once complete, another set of washing steps is performed, and slides are spun dry at 1200 rpm for 2 minutes and stored once again in the dessicator.

DNA Preparation.

PCR products are prepared from clinical samples. This DNA to be used for hybridization is then purified using the Qiagen PCR purification kit prior to labeling. Each DNA is run on a 2% agarose gel with Invitrogen's Low Mass DNA Ladder to determine its approximate concentration.

PCR and Cy3-dCTP Labeling.

The PCR/labeling step is carried out using Invitrogen's high fidelity ThermalAce DNA Polymerase.

The Master Mix containing the Cy3-dCTP must be kept out of the light, and covered with tin foil.

The protocol is as follows for one reaction:

| | | |
|---|---|---|
| 10× ThermalAce Buffer | 5 | ul |
| 2 mM dATP, dGTP, dTTP | 2.5 | ul |
| 1 mM Cy3-dCTP (Amersham):1 | 5 | ul |
| mM unlabeled dCTP in a 1:10 solution | | |
| 10% TritionX-100 | 0.5 | ul |
| 10 uM 16S Forward primer | 2 | ul |
| 10 uM 16S Reverse primer | 2 | ul |
| ThermalAce Polymerase | 1 | ul |
| 70 ng of DNA | x | ul |

ddH₂0 to 50 ul
95° C. 4 min.
95° C. 45 s, 53° C. 45 s, 72° C. 1:30 (20 cycles)
72° C. 15 min.
4° C. forever

PCR products can be stored at 4° C. wrapped in tin foil until ready for purification.
PCR Purification.

Cy3-labeled PCR products are purified using a supplementary protocol with the QIAquick PCR Purification Kit.

In this protocol, an extra wash is performed using a 35% guanidine hydrochloride aqueous solution, which helps to purify DNA fragments from CyDye-labeled reactions. Samples are eluted in elution buffer and stored at 4° C. wrapped in foil until ready for hybridization.

Hybridization Cocktail.

A hybridization solution is prepared using 8 ul of the purified labeled DNA, 1 ul 20×SSC (2× final conc.), 1 ul yeast tRNA (10 ug/ul), 0.5 ul HEPES pH 7.0 and 0.25 ul 10% SDS (0.1% final). The solution is mixed and spun briefly in a centrifuge, then denatured at 100° C. for 2 min.

Hybridization.

The blocked slide and an Incyte Hybridization cassette are cleaned off using a nitrogen gas line. The slide is then placed in the chamber array side up. 9×18 mm glass ‘Lifterslips’ custom ordered from Erie Scientific are then placed over each of the arrays on the slide using forceps. These cover slips are also cleaned using the nitrogen gas to remove dust.

125 ul of 3×SSC is added to the two wells of the hybridization cassette, to help prevent the slide from drying out during the incubation. The 10 ul of denatured hybridization cocktail is then carefully injected under its respective cover slip using a pipette, until the entire area is filled. The hybridization cassette is sealed, and placed in a 55° C. water bath for 5-16 h.

http://www.eriemicroarray.com/coverglass/lifterslips-st.aspx

Post-Processing.

The array is removed from the water bath. Slides are washed as follows:

Wash 1: 2×SSC+0.2% SDS solution, 55° C., 100 dunks

Wash 2: 2×SSC solution, room temp, 100 dunks

Wash 3: 0.2×SSC solution, room temp., 100 dunks.

The slides are then spun dry in a slide rack at 1200 rpm for 2 minutes then stored in a foil-covered slide box.

Scanning.

A GenePix 4000B microarray scanner and GenePix Pro software are used for scanning the slides.

Slides are placed array-side down into the scanner and scanned one at a time using a wavelength of 532 nm to visualize the Cy3 label. The PMT (photomultiplier tube) is set at 600.

Median pixel intensities for each individual spot can be calculated using the analysis function of the software. The background intensity is subtracted from that score—yielding a “median intensity score” for each individual spot.

This score allows for the determination of the presence or absence of a particular microorganism based on specific criteria set for that individual spot. (The set criteria are established during careful validation studies for each spot's performance).

A DNA probe from a pure culture hybridizes only to its anticipated target as seen in FIG. 2. Hybridization of DNA from healthy and diseased clinical samples are shown FIG. 3.

http://www.axon.com/gn_GenePix4000.html

### Example 2

### Reproducibility Data for Human Oral Microbial Identification Microarray

A pool of bacterial cells were taken, and the DNA was lysed in 1 reaction.

Experiment #1=Reproducibility of data from the arrays within 1 slide:

There were 5 arrays printed on each of the slides. The 5 arrays were printed identically, and 5 different hybridizations per slide were performed by covering each of the 5 separate arrays with its own cover slip, and then injecting 5 separate samples under it's respective coverslip. The 5 arrays on the 1 slide can be seen in FIG. 7. FIG. 7 is a photograph showing an image of the low resolution initial scan of the entire slide (Arrays #1-5, Slide #30), showing five individual arrays printed on the slide. Each was hybridized with labeled product from the same starting template (amplified as 5 separate reactions).

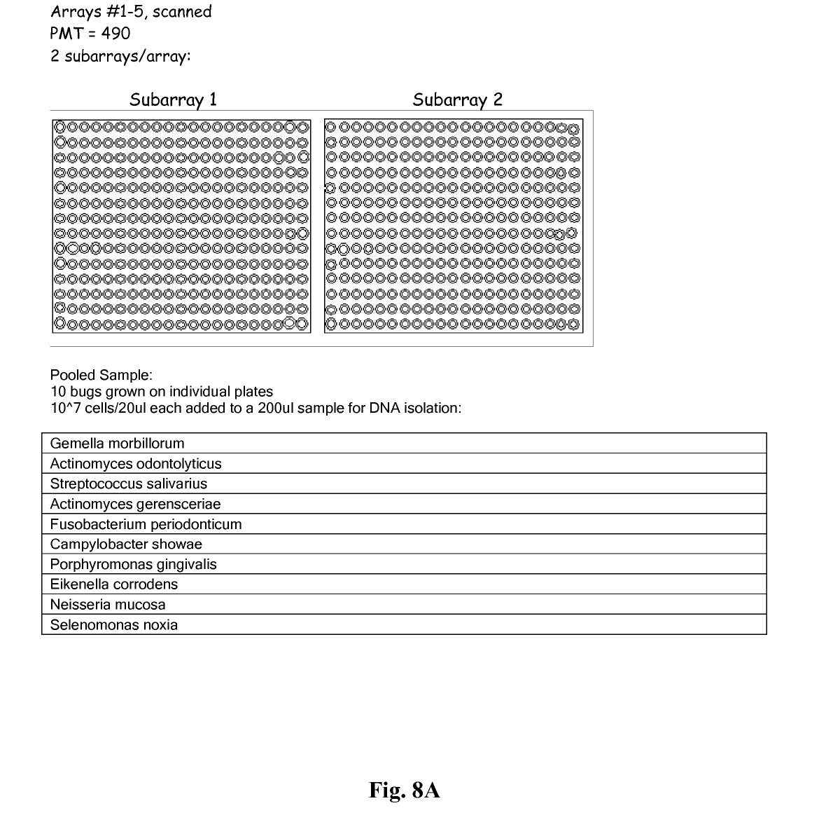

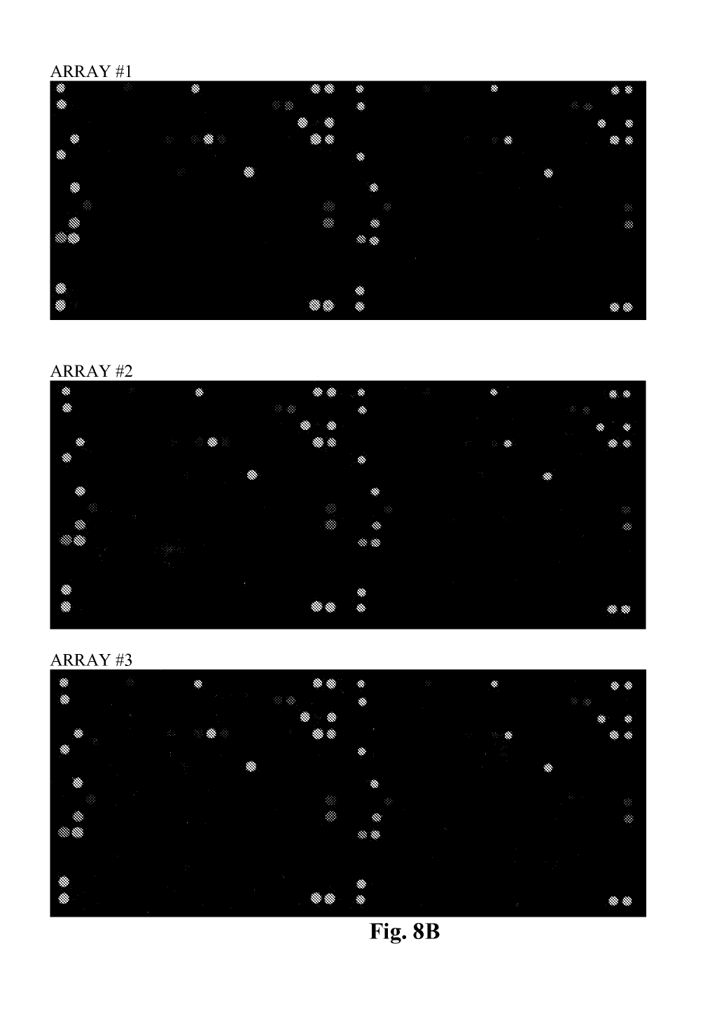

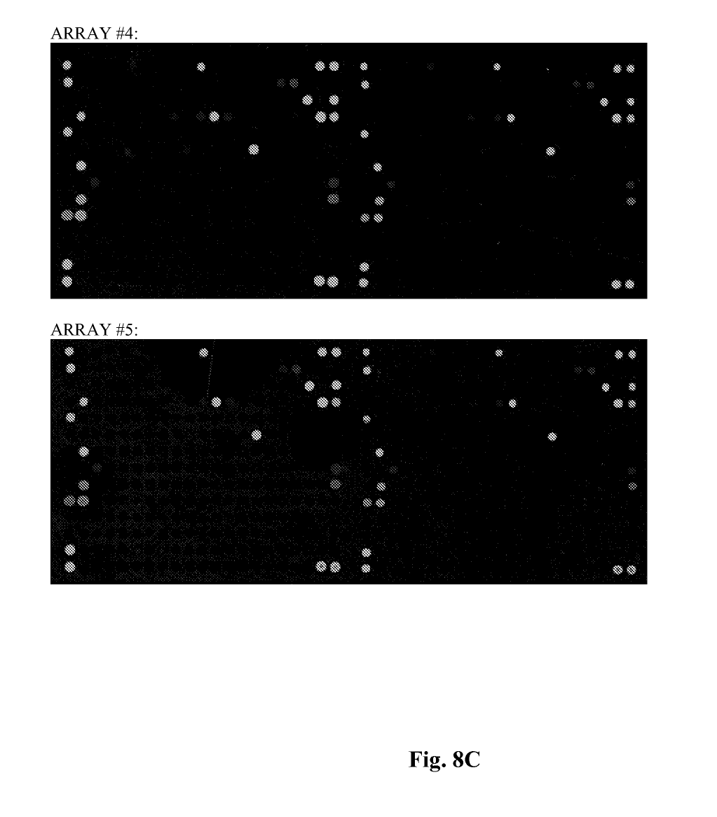

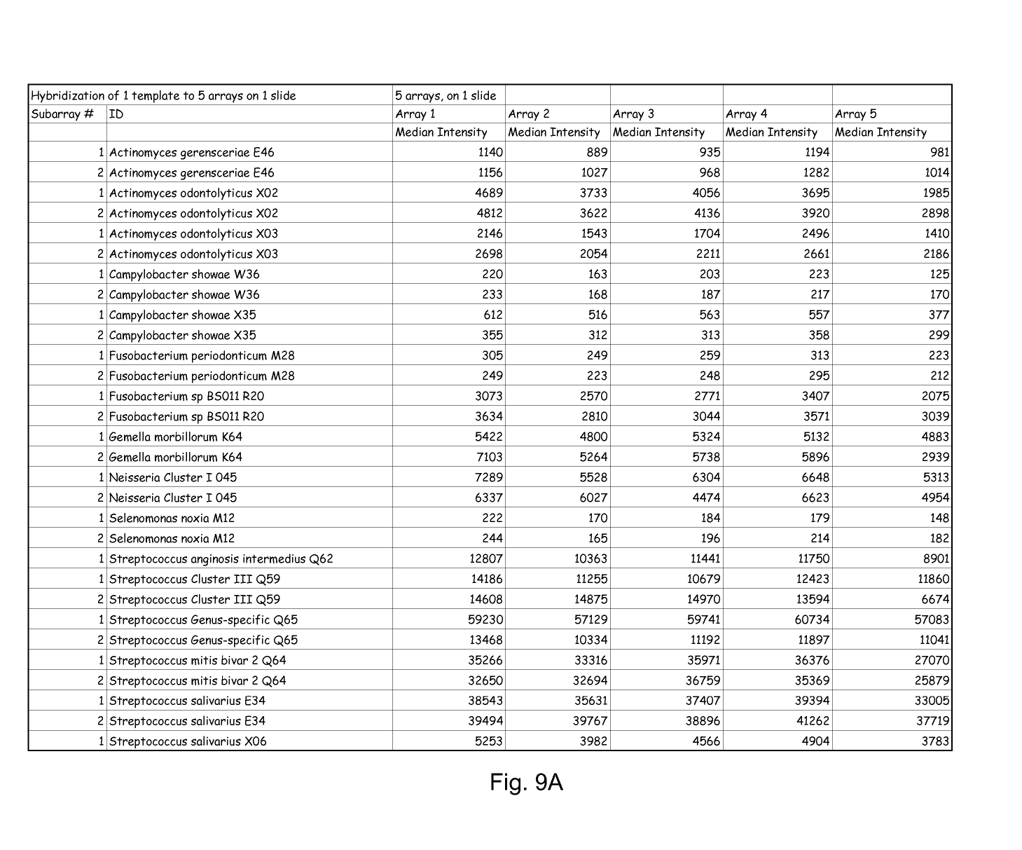

FIG. 8A shows the reproducibility between the 2 sub-arrays for each whole array (2 subarrays underneath 1 coverslip). (Arrays #1-5, scanned; PMT=490; 2 subarrays/array). A pool sample was used and consisted of ten bacteria (Gemella morbillorum, Actinomyces odontolyticus, Streptococcus salivarius, Actinomyces gerensceriae, Fusobacterium periodonticum, Campylobacter showae, Porphyromonas gingivalis, Eikenella corrodens, Neisseria mucosa, and Selenomonas noxia) were grown on individual plates (10⁷ cells/20 ul each added to a 200 ul sample for DNA isolation). FIGS. 8B-8C show a high-resolution scan of 5 individual arrays, each hybridized with labeled product from the same starting template (amplified as 5 separate reactions). FIGS. 8B-8C show that hybridization results for 1 sample are reproducible between arrays on one individual slide and reproducible results are also seen between subarrays under the same coverslip.

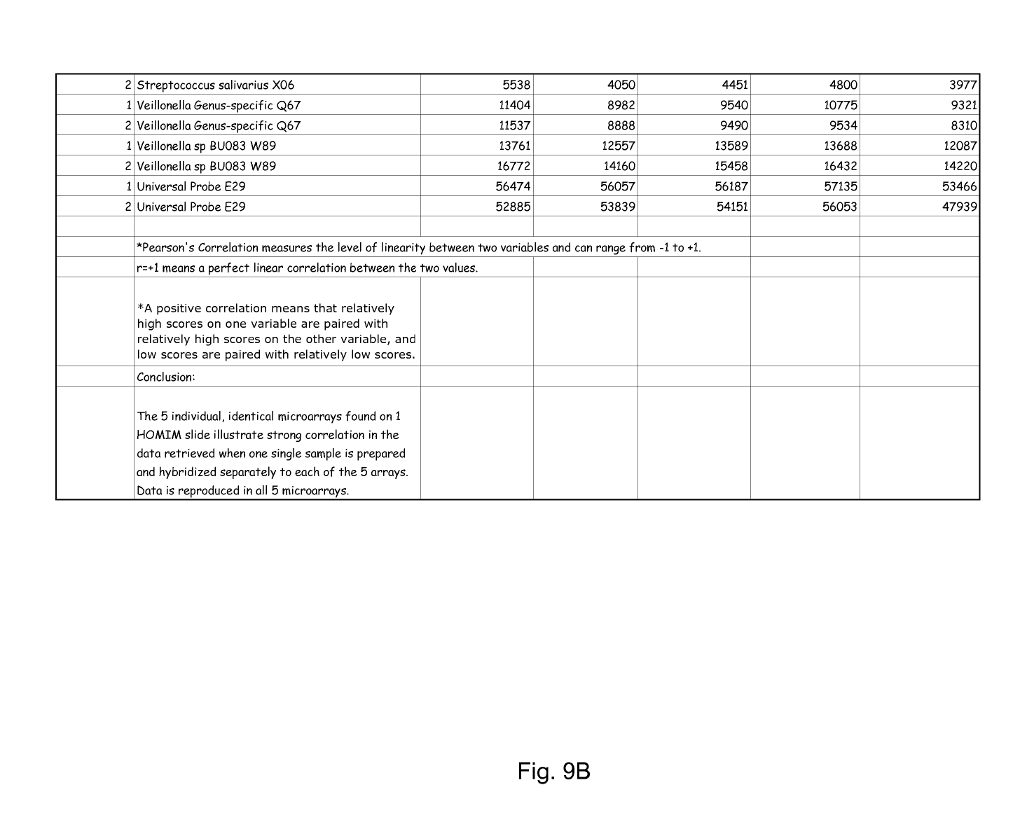

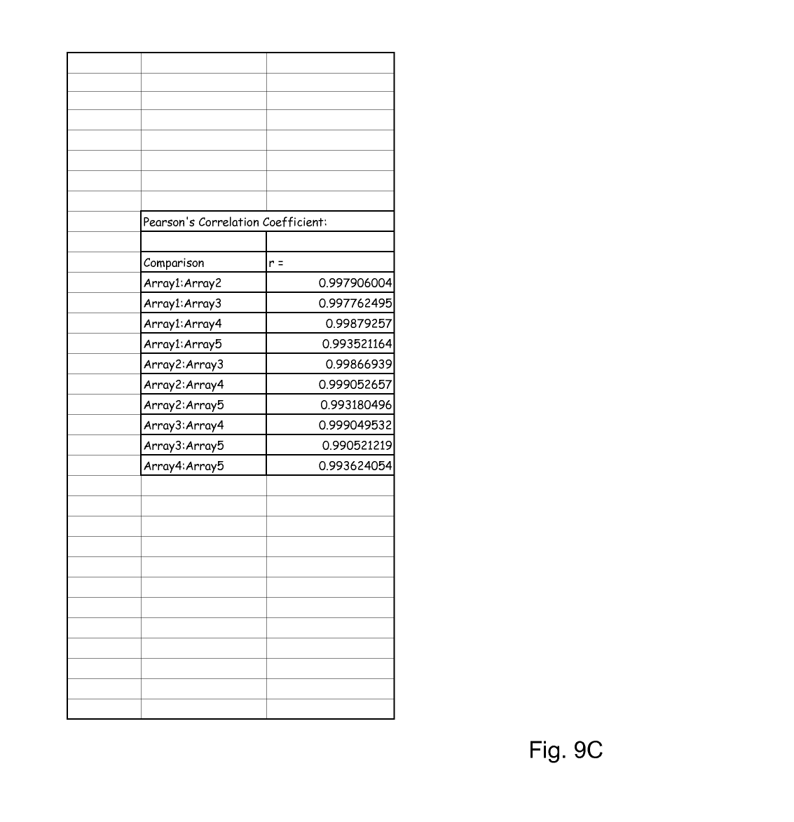

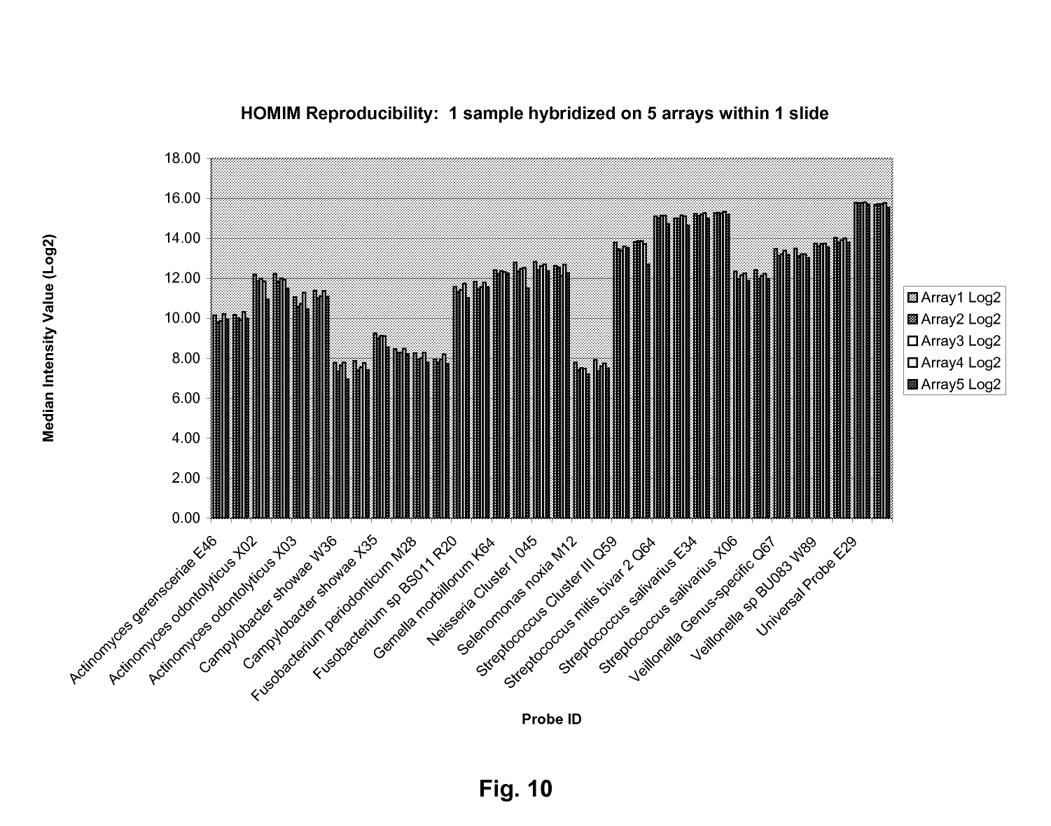

This lysed DNA template, above, was taken, and 5 separate reactions were prepared to yield 5 sets of identical labeled DNA. Each one of the 5 was hybridized to its own array on the 1 slide, and then scanned and analyzed the data. FIG. 9A-C is a table that shows the array to array reproducibility results and lists the intensity values extracted from all 5 arrays. In particular, FIG. 9A-C compares median intensity scores (with background intensity subtraction) for the spots across all 5 individual arrays within 1 slide. FIG. 10 shows the data from FIG. 9A-C in graph form and shows that all 5 arrays on the one slide had reproducibility; the values for each ‘positive’ spot were comparable. Consistency in fluorescent values between arrays hybridized with the same sample on the same slide is shown.

Experiment #2=Reproducibility of Data from Arrays on 2 Different Slides:

The same methods described for Experiment #1 were performed, except 2 labeled samples from identical DNA template were prepared. One sample was hybridized under a coverslip on one slide, and one sample under a cover slip on a second slide.

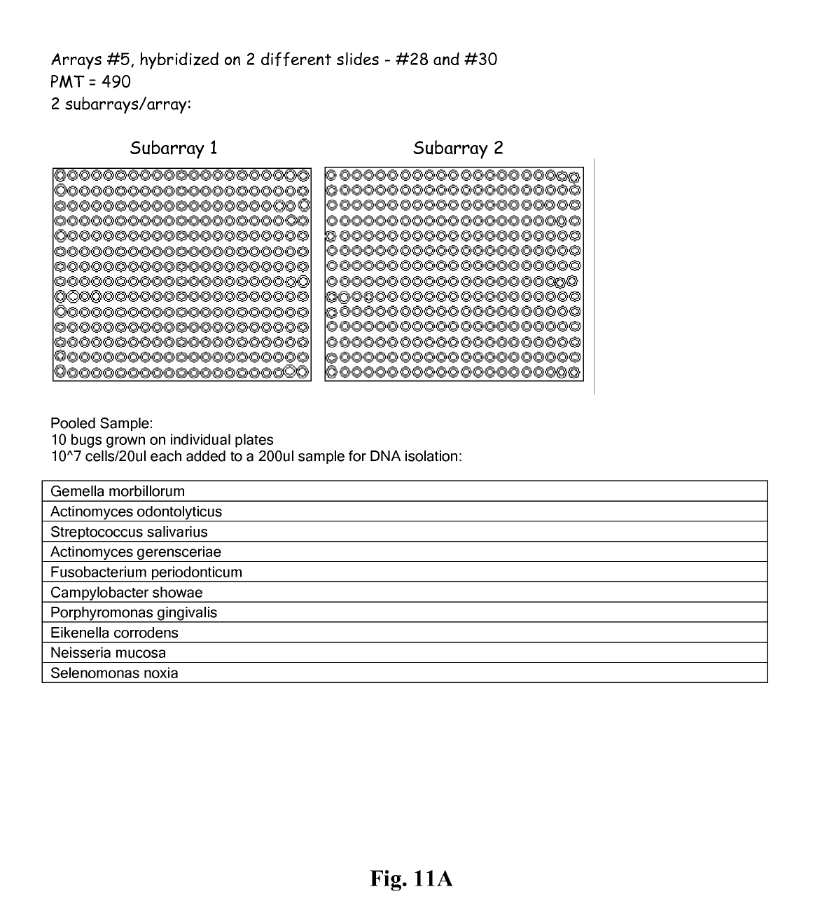

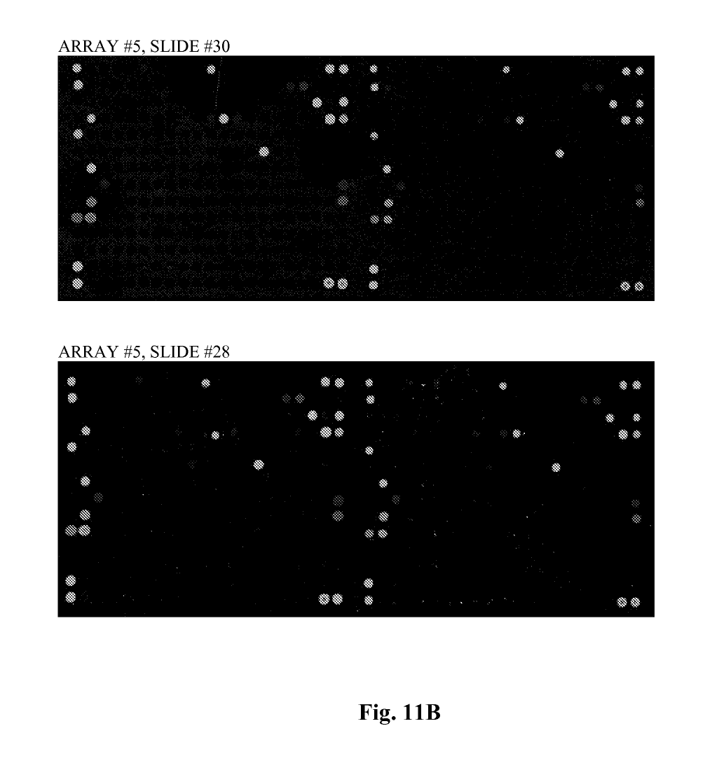

FIG. 11A depicts shows the reproducibility between 2 individual arrays hybridized on different slides with labeled DNA from the same starting template (amplified as 2 separate reactions). (Array #5, hybridized on 2 different slides—#28 and #30; PMT=490; 2 subarrays/array). A pool sample was used and consisted of ten bacteria (Gemella morbillorum, Actinomyces odontolyticus, Streptococcus salivarius, Actinomyces gerensceriae, Fusobacterium periodonticum, Campylobacter showae, Porphyromonas gingivalis, Eikenella corrodens, Neisseria mucosa, and Selenomonas noxia) were grown on individual plates (10⁷ cells/20 ul each added to a 200 ul sample for DNA isolation). FIG. 11B shows show a high-resolution scan of hybridization results for 1 sample between 2 different slides. FIG. 11B shows that the hybridization results are reproducible between 2 different slides, and between subarrays under the same coverslip.

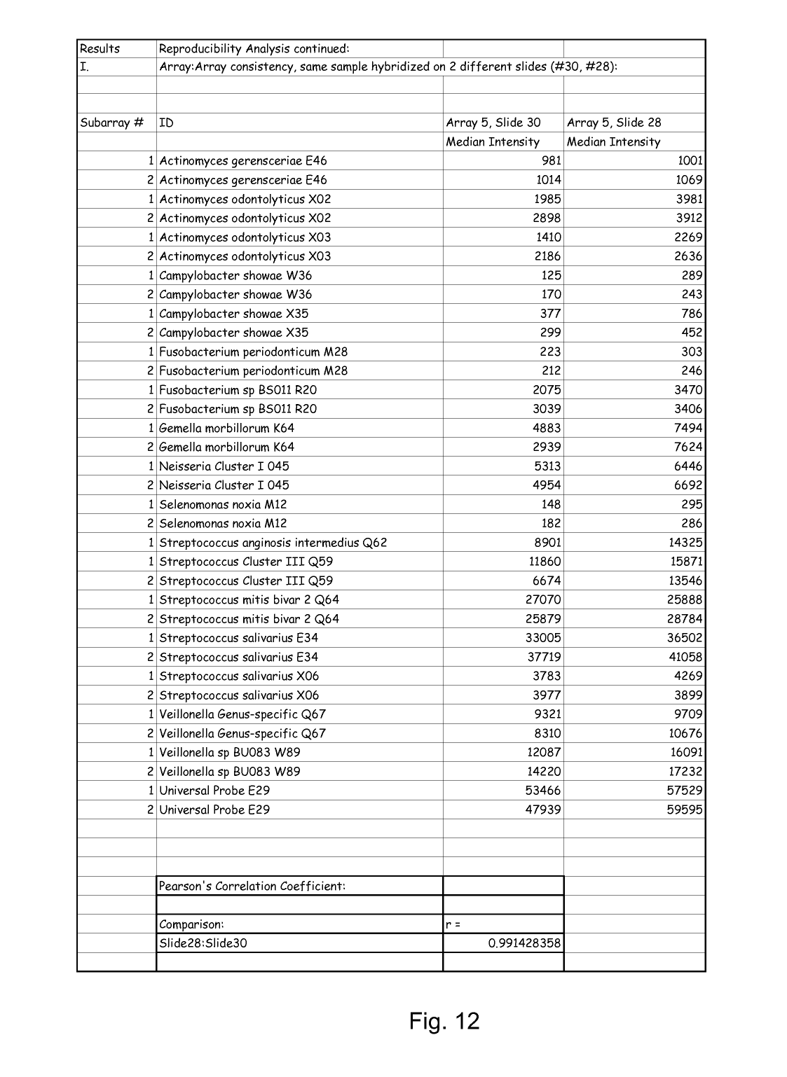

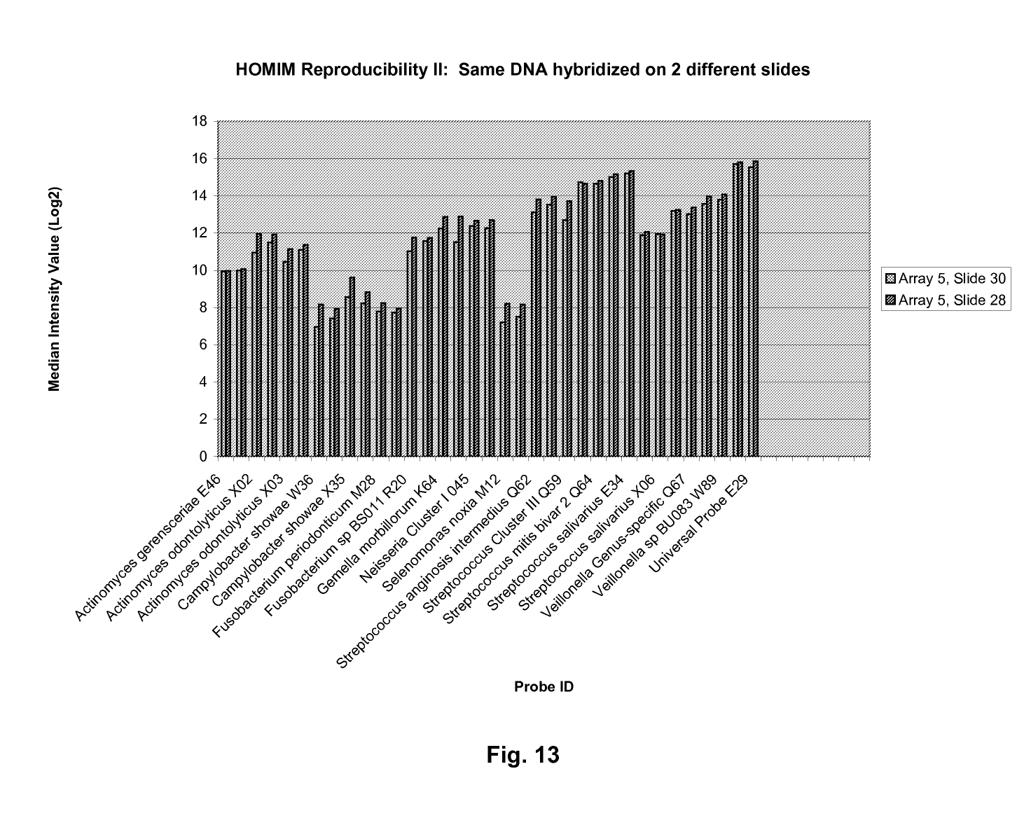

FIG. 12 shows a table of reproducibility data between arrays. FIG. 12 lists the intensity values extracted from the 1 array on one slide, and the other array on the 2nd slide, and shows that even when hybridized on 2 separate slides, the values extracted are comparable. FIG. 13 is a graph of the reproducibility data shown in the table in FIG. 12. FIGS. 12 and 13 compare median intensity scores (with background subtraction) for the 2 arrays illustrated above, and illustrates consistency in fluorescent values between arrays hybridized with the same sample on 2 different slides.

The median intensity score is the calculated median score taken from all pixel intensity scores found within one spot.

Intensity Data:

Each individual spot on the array was treated as a separate entity by the analysis software. Each spot contained around 150 pixels, and the software recorded the fluorescence of each pixel for a particular spot and then calculated the median intensity. The software also calculated a ‘local background’ where it calculated the pixels in a certain area surrounding the 1 particular spot. A median background intensity is also calculated, and this background value is subtracted from the median intensity value of the spot, giving a semi-normalized ‘spot fluorescence value’. This value is referred to herein as the “median intensity value”.

Once this medial intensity value is obtained, a set of criteria to analyzed the data was applied. These criteria determine whether or not a bacteria is “present” or “absent”.

HOMIM Analysis Steps/Criteria:

Consideration:

At least N=4 was used for each experiment if not more (2 duplicate arrays), and all data was analyzed together. This step helps determine which data is real, and which are flukes.

Initial Normalization procedure, which corrects for fluorescent background:

1. GenePix-calculated local median background (B532) was subtracted from the median pixel intensity for each individual spot (F532-B532). This is the value that was utilized as the “Median Intensity Score”.

Since only one-color arrays were used, a ratio R/G normalization was not needed.

Analysis:

2. List was sorted by Normalized Intensity Values. All values that fall below the background median and all flagged spots were removed.

3. All median intensity scores that were ≦2× median background were removed.

4. Signal intensity >2× the standard deviation for the spot=95% intensity confidence was determined because any spots not meeting this criteria may not be reliable. Removed all spots ≦2× SD.

5. Data was sorted by signal to noise ratio, and all those that fall SNR<1.5 were removed. Confidence increases as noise variation (bg signal) decreases.

The signal S that is generated by the photomultiplier tube for each pixel is a function of the number N of incident emission photons times the quantum efficiency Q_{E} of the photomultiplier tube. Therefore S═N×Q_{E}. N is the result of not only an efficient light collection pathway, but also an efficient laser excitation pathway.

Signal-to-noise is used for determining the confidence with which one can quantify a signal peak of a given value, especially a signal near background. The confidence in quantifying the peak increases as the variation in background signal (i.e. the noise) decreases, regardless of the absolute average background.

6. A log2 value was calculated for each intensity value for analysis and graphing purposes.

The relevant teachings of all the references, papers, journal articles, patents and/or patent applications cited herein are incorporated herein by reference in their entirety.

While this invention has been particularly shown and described with references to preferred embodiments thereof, it will be understood by those skilled in the art that various changes in form and details may be made therein without departing from the scope of the invention encompassed by the appended claims.

What is claimed is:

1. A method for identifying one or more microorganisms in a sample from an individual, wherein the method comprises:
a. contacting nucleic acid molecules obtained from the sample with one or more nucleic acid molecules having a nucleic acid sequence selected from the group consisting of:
i. any one of SEQ ID NOs:1-295;
ii. a nucleic acid sequence having between about 80% and about 100% of contiguous nucleotides of any one of SEQ ID NO: 1-295;
iii. any one of SEQ ID NOs:296-585;
iv. a nucleic acid sequence having between about 15 and about 25 contiguous nucleotides of any one of SEQ ID NO: 296-585; and
v. any combination thereof;
under conditions suitable for hybridization to thereby form a complex; and
b. detecting the presence or absence of the complex;
wherein the presence of the complex indicates the presence of the microorganism in the sample and the absence of the complex indicates the absence of the microorganism in the sample, and wherein the microorganism identified is at least one microorganism selected from the group consisting of: Actinobacillus actinomycetemcomitans, Actinobaculum sp. EL030, Actinomyces georgiae, Actinomyces gerensceriae, Actinomyces naeslundii I, Actinomyces naeslundii II, Actinomyces odontolyticus, Actinomyces sp. AP064, Actinomyces sp. B19SC, Actinomyces sp. B27SC, Actinomyces sp. EP005, Actinomyces sp. EP011, Actinomyces sp. EP053, Actinomyces israelii, Atopobium parvulum, Atopobium rimae, Atopobium sp. C019, Tannerella forsythia, Tannerella forsythia, Bacteroidetes sp. _X083, Bacteroidetes sp. AU126, Bifidobacterium (genus-specific), Bifidobacterium dentium, Bifidobacterium sp. strain A32ED, Bifidobacterium sp. CX010, Brevundimonas diminuta, Bulledia extructa/Solobacterium moorei, Campylobacter concisus, Campylobacter gracilis, Campylobacter rectus/concisus, Campylobacter cluster: (C. rectus/showae/curvus), Campylobacter showae, Capnocytophaga ochracea/sp. BB167, Capnocytophaga sp. _X066, Capnocytophaga sp. _X089, Capnocytophaga sp. AA032, Capnocytophaga sp. BB167, Capnocytophaga cluster: (C. ochracea/sp. BM058/BU084/DZ074/BR085, Capnocytophaga sp. BR085, Capnocytophaga sp. DS022, Capnocytophaga gingivalis/sp. S3, Capnocytophaga sputigena, Cardiobacterium hominis, Corynebacterium durum, Corynebacterium matruchotii, Cryptobacterium curtum, Desulfobulbus sp. _R004/CH031, Dialister invisus, Dialister pneumosintes, Eikenella corrodens, Eubacterium brachy, Eubacterium infirmum, Eubacterium nodatum, Eubacterium sp. IR009, Eubacterium saphenum, Eubacterium sp. strain A3MT, Eubacterium sp. BB124, Eubacterium sp. BB142, Eubacterium sp. DO008, Eubacterium sulci, Eubacterium yurii, Filifactor alocis, Fusobacterium sp. _I035, Fusobacterium cluster: (F. nucleatum/CZ006/_R002/ss. vincentii/naviforme), Fusobacterium nucleatum ss. nucleatum, Fusobacterium nucleatum ss. polymorphum, Fusobacterium periodonticum, Fusobacterium sp. BS011, Gemella haemolysans, Gemella morbillorum, Granulicatella adicens/elegans, Haemophilus influenzae, Haemophilus parainfluenzae/paraphrophilus, Haemophilus paraphrophaemolyticus/sp. BJ021, Haemophilus segnis, Haemophilus sp. BJ095, Kingella denitrificans, Kingella oralis, Lactobacillus casei/rhamnosis/zeae, Lactobacillus fermentum, Lactobacillus gasseri, Lactobacillus sp. CX036 vaginalis, Lactobacillus sp. HT070, Lautropia mirabilis, Lautropia sp. AP009, Leptotrichia buccalis, Leptotrichia hofstadii, Leptotrichia sp. DR011, Leptotrichia sp. FB074/BB002, Leptotrichia sp. GT018, Leptotrichia wadei, Megasphaera sp. BB166, Megasphaera sp. BU057, Megasphaera sp. CS025, Micromonas micros, Micromonas cluster: M. micros/FG014/BS044, Micromonas sp. DA014, Mycoplasma faucium, Mycoplasma hominis, Mycoplasma salivarium, Neisseria elongata, Neisseria cluster I: N. elongata/sp. AP015/Eikenella corrodens, Neisseria flavescens, Neisseria cluster II: (N. mucosa/sicca/flava/AP015), Neisseria pharyngis, Neisseria cluster III: (N. polysaccharea/gonorrhoeae/meningitides), Neisseria bacilliformis/sp. AP132, Neisseria mucosa/sp. AP060, Neisseria sp. strain B33KA, Olsenella genomospecies C1, Peptostreptococcus sp. CK035, Porphyromonas catoniae, Porphyromonas endodontalis cluster: (P. endodontalis/F016/BB134/AJ002), Porphyromonas gingivalis, Porphyromonas sp. BB134, Porphyromonas cluster: (sp. BR037/DP023/EP003), Porphyromonas sp. CW034/DS023, Porphyromonas sp. CW034/DS033, Porphyromonas sp. DP023, Prevotella buccae, Prevotella (Bacteroides) heparinolytica, Prevotella intermedia, Prevotella loeschii/GU027, Prevotella cluster I: (P. loeschii/GU027/B31FD, Prevotella melaminogenica, Prevotella nigrescens, Prevotella oralis, Prevotella oris/_F045, Prevotella oulora, Prevotella pallens, Prevotella cluster II: (P. denticola/sp. AH005/AO036), Prevotella denticola/sp. AH005, Prevotella sp. AH125, Prevotella sp. BE073, Prevotella sp. BI027, Prevotella sp. CY006/FL019, Prevotella sp. DO027, Prevotella sp. DO039, Prevotella sp. DO045, Prevotella sp. DO022, Prevotella sp. FM005, Prevotella sp. HF050, Prevotella tannerae, Propionibacterium acnes, Propionibacterium sp. strain FMA5, Pseudomonas aeruginosa, Rhodocyclus sp. strain A08KA, Rothia dentocariosa, Rothia dentocariosa/mucilaginosa, Selenomonas dianae, Selenomonas flueggii, Selenomonas infelix, Selenomonas noxia, Selenomonas sp. AA024, Selenomonas sp. AH132, Selenomonas sp. AJ036, Selenomonas sp. CI002, Selenomonas sp. CS002, Selenomonas sp. CS015, Selenomonas sp. CS024, Selenomonas sp. DD020, Selenomonas sp. DM071, Selenomonas sp. EZ011, Selenomonas sp. DS051, Selenomonas sp. EW076, Selenomonas sp. EW079/JS031, Selenomonas sp. EW084/DS071, Selenomonas sputigena, Streptococcus (genus-specific), Streptococcus anginosus/gordonii, Streptococcus anginosus/intermedius, Streptococcus constellatus/intermedius, Streptococcus cristatus, Streptococcus cluster I: (S. gordonii/anginosus/mitis, Streptococcus infantis/sp.FN042, Streptococcus mitis biovar 2, Streptococcus cluster II: (S. mitis/oralis/pneumoniae), Streptococcus mutans, Streptococcus parasanguinis, Streptococcus salivarius, Streptococcus cluster III: (S. sanguinis/salivarius/mitis/C3, Streptococcus australis, Streptococcus cluster IV: sp. C6/C3/P4/7A, Stretococcus sobrinus, Synergistes (Phylum-specific, Synergistes sp. _D084, Synergistes sp. _W028, Synergistes sp. _W090, Synergistes sp. BB062, Synergistes sp. BH017, Tannerella sp. BU063, TM7 sp. _I025, TM7 sp. AH040, TM7 sp. BE109, TM7 sp. BE109/BU080, Treponema 08:A:pectinovorum, Treponema (genus specific), Treponema denticola, Treponema lecithinolyticum, Treponema medium, Treponema socranskii (all subspecies), Treponema sp. AT039, Treponema vincentii, Veillonella dispar/_X042/, Veillonella (genus-specific), Veillonella atypica, Veillonella parvula, Veillonella sp. AA050/_X042, Veillonella sp. BU083, and Escherichia coli.

2. The method of claim 1, wherein identifying one or more microorganisms includes detecting nucleic acid of the microorganism.

3. The method of claim 2, wherein detecting nucleic acid of the microorganism includes detecting 16S rRNA of the microorganism.

4. The method of claim 1, wherein the sample from the individual is obtained from the group consisting of the oral cavity, sinus, esophagus, respiratory tract, lungs, sputum, pharynx, eustachian tube, middle ear, vagina, blood, pus, spinal fluid, and gastrointestinal tract.

5. The method of claim 1, wherein the presence of a single microorganism is identified by the presence of at least two different complexes between the nucleic acid molecule and the sample.

6. The method of claim 1, further comprising labeling the nucleic acid molecules of the sample with a detectable label.

7. The method of claim 6, wherein the detectable label is selected from the group consisting of fluorescent dyes, streptavidin conjugate, magnetic beads, dendrimers, radiolabels, enzymes, calorimetric labels, nanoparticles, and nanocrystals.

8. The method of claim 1, wherein the nucleic acid are bound to a solid support.

9. The method of claim 8, wherein the solid support is selected from the group consisting of glass, silica chips, nylon membrane, polymer, plastic, ceramic, metal, and optical fiber.

10. A method of assessing the compositional flora of microorganisms from a sample of an individual; the method comprises:
a. contacting nucleic acid molecules obtained from the sample with one or more nucleic acid molecules having a nucleic acid sequence selected from the group consisting of:
i. any one of SEQ ID NOs:1-295;
ii. a nucleic acid sequence having between about 80% and about 100% of contiguous nucleotides of any one of SEQ ID NO: 1-295;
iii. any one of SEQ ID NOs:296-585;
iv. a nucleic acid sequence having between about 15 and about 25 contiguous nucleotides of any one of SEQ ID NO: 296-585; and
v. any combination thereof;
under conditions suitable for hybridization to thereby form a complex; and
b. detecting the presence or absence of the complex; wherein the presence of one or more complexes indicates the presence of one or more microorganisms recited in claim 1 and the absence of one or more complexes indicates the absence of one or more microorganisms recited in claim 1;
wherein the compositional flora is composed of the presence, absence, or both of one or more microorganisms.

11. A method for diagnosing an individual having a disease or condition, the method comprises:
determining the presence, absence, level or percentage of one or more nucleic acid molecules from a sample from the individual that hybridize to one or more nucleic acid molecules having a nucleic acid sequence selected from the group consisting of:
i. any one of SEQ ID NOs:1-295;
ii. a nucleic acid sequence having between about 80% and about 100% of contiguous nucleotides of any one of SEQ ID NO: 1-295;
iii. any one of SEQ ID NOs:296-585;
iv. a nucleic acid sequence having between about 15 and about 25 contiguous nucleotides of any one of SEQ ID NO: 296-585; and
v. any combination thereof;
wherein the presence, absence, level or percentage of one or more complexes indicates the presence, absence, or severity of the disease or condition.

12. A method for diagnosing an individual having a disease or condition, the method comprises:
a. contacting nucleic acid molecules obtained from the sample with one or more nucleic acid molecules having a nucleic acid sequence selected from the group consisting of:
i. any one of SEQ ID NOs:1-295;
ii. a nucleic acid sequence having between about 80% and about 100% of contiguous nucleotides of any one of SEQ ID NO: 1-295;
iii. any one of SEQ ID NOs:296-585;
iv. a nucleic acid sequence having between about 15 and about 25 contiguous nucleotides of any one of SEQ ID NO: 296-585; and
v. any combination thereof;
under conditions suitable for hybridization to thereby form a complex; and
b. detecting the presence or absence of the complex, wherein the presence of one or more complexes indicates the presence of one or more microorganisms recited in claim 1 and the absence of one or more complexes indicates the absence of one or more microorganisms recited in claim 1;
wherein the presence or absence of one or more microorganisms indicates the presence or absence of the disease or condition.

13. The method of claim 12, wherein the disease or condition is periodontal disease.

14. A method for providing a prognosis for an individual having a disease or condition, the method comprises:
determining the presence, absence, level or percentage of one or more nucleic acid molecules from a sample from the individual that hybridize to one or more nucleic acid molecules having a nucleic acid sequence selected from the group consisting of:
i. any one of SEQ ID NOs:1-295;
ii. a nucleic acid sequence having between about 80% and about 100% of contiguous nucleotides of any one of SEQ ID NO: 1-295;
iii. any one of SEQ ID NOs:296-585;
iv. a nucleic acid sequence having between about 15 and about 25 contiguous nucleotides of any one of SEQ ID NO: 296-585; and
v. any combination thereof;
wherein the presence, absence, level or percentage of one or more complexes indicates the prognosis of the disease or condition.

15. The method of claim 14, wherein the disease or condition is selected from the group consisting of periodontal disease, alveolar osteoitis, caries, oral cancer, diabetes, AIDS, Sjögren's syndrome, smoking and alcoholism.

16. A method for monitoring treatment or efficacy of therapy for an individual having a disease or condition, the method comprises:
a. determining the presence, absence, level or percentage of one or more nucleic acid molecules from a sample from the individual that hybridize to one or more nucleic acid molecules having a nucleic acid sequence selected from the group consisting of:
i. any one of SEQ ID NOs:1-295;
ii. a nucleic acid sequence having between about 80% and about 100% of contiguous nucleotides of any one of SEQ ID NO: 1-295;
iii. any one of SEQ ID NOs:296-585;
iv. a nucleic acid sequence having between about 15 and about 25 contiguous nucleotides of any one of SEQ ID NO: 296-585; and
v. any combination thereof;
at one or more time points; and
b. comparing or analyzing the presence, absence, level or percentage of the one or more complexes at the one or more time points;
wherein said comparison or analysis indicates the efficacy of therapy.

17. The method of claim 16, wherein the therapy is selected from the group consisting of antibiotic therapy, surgery, and administration of medication.

18. A method for monitoring the effect of an oral product on the oral microflora an individual, the method comprises:
a. determining the presence, absence, level or percentage of one or more nucleic acid molecules from a sample from the individual that hybridize to one or more nucleic acid molecules having a nucleic acid sequence selected from the group consisting of:
i. any one of SEQ ID NOs:1-295;
ii. a nucleic acid sequence having between about 80% and about 100% of contiguous nucleotides of any one of SEQ ID NO: 1-295;
iii. any one of SEQ ID NOs:296-585;
iv. a nucleic acid sequence having between about 15 and about 25 contiguous nucleotides of any one of SEQ ID NO: 296-585; and
v. any combination thereof;
at one or more time points, wherein at least time point occurs after administration of said oral product; and
b. comparing or analyzing the presence, absence, level or percentage of the one or more complexes at the one or more time points;
wherein said comparison or analysis indicates the efficacy of the oral product.

19. The method of claim 18, wherein the oral product is selected from the group consisting of toothpaste, mouthwash, fluoride, breath enhancers, tooth-whitening treatments, floss, and the like.

20. The method of claim 18, wherein the individual has an oral or extraoral systemic disease or condition.

21. The method of determining the presence or absence of one or more microorganisms recited in claim 1, the method comprises detecting the presence of one or more nucleic acid molecules having a nucleic acid sequence selected from the group consisting of:
a. any one of SEQ ID NOs:1-295;
b. a nucleic acid sequence having between about 80% and about 100% of contiguous nucleotides of any one of SEQ ID NO: 1-295;
c. any one of SEQ ID NOs:296-585;
d. a nucleic acid sequence having between about 15 and about 25 contiguous nucleotides of any one of SEQ ID NO: 296-585;
e. a reverse complement of a-d; and
f. any combination thereof;
wherein the presence of the nucleic acid molecules indicates the presence of the microorganism, and the absence of the nucleic acid molecules indicates the absence of the microorganism.

22. A method for identifying one or more unnamed or uncultivated microorganisms in a sample from an individual, the method comprises:
a. contacting nucleic acid molecules obtained from the sample with one or more nucleic acid molecules having a nucleic acid sequence selected from the group consisting of:
i. any one of SEQ ID NOs:1-295;
ii. a nucleic acid sequence having between about 80% and about 100% of contiguous nucleotides of any one of SEQ ID NO: 1-295;
iii. any one of SEQ ID NOs:296-585;
iv. a nucleic acid sequence having between about 15 and about 25 contiguous nucleotides of any one of SEQ ID NO: 296-585; and
v. any combination thereof;
under conditions suitable for hybridization to thereby form a complex; and
b. detecting the presence or absence of the complex;
wherein the presence of the complex indicates the presence of the microorganism in the sample and the absence of the complex indicates the absence of the microorganism in the sample, and wherein the nucleic acid molecules having at least one of the sequence of SEQ ID Nos:1-585 is identical to a non-conserved region of nucleic acid sequence from the unnamed or uncultivated microorganism.

23. A method for identifying a microorganism in a sample from an individual, the method comprises:
a. amplifying and labeling DNA from the sample with a detectable label using a Polymerase Chain Reaction (PCR);
b. contacting nucleic acid molecules obtained from the sample with one or more nucleic acid molecules having a nucleic acid sequence selected from the group consisting of:
i. any one of SEQ ID NOs:1-295;
ii. a nucleic acid sequence having between about 80% and about 100% of contiguous nucleotides of any one of SEQ ID NO: 1-295;
iii. any one of SEQ ID NOs:296-585;
iv. a nucleic acid sequence having between about 15 and about 25 contiguous nucleotides of any one of SEQ ID NO: 296-585; and
v. any combination thereof;
under conditions suitable for hybridization to thereby form a complex; and
c. detecting the presence or absence of the complex;
wherein the presence of a complex indicates the presence of the microorganism recited in claim 1 and the absence a complex indicates the absence of the microorganism recited in claim 1.

24. A method for identifying a microorganism in a sample from an individual, the method comprises:
a. reverse transcribing RNA obtained from the sample to thereby obtain DNA;
b. optionally amplifying the DNA by PCR;
c. labeling the DNA;
d. contacting DNA obtained from the sample with one or more nucleic acid molecules having a nucleic acid sequence selected from the group consisting of:
i. any one of SEQ ID NOs:1-295;
ii. a nucleic acid sequence having between about 80% and about 100% of contiguous nucleotides of any one of SEQ ID NO: 1-295;
iii. any one of SEQ ID NOs:296-585;
iv. a nucleic acid sequence having between about 15 and about 25 contiguous nucleotides of any one of SEQ ID NO: 296-585; and
v. any combination thereof;
under conditions suitable for hybridization to thereby form a complex; and
e. detecting the presence or absence of the complex;
wherein the presence of a complex indicates the presence of the microorganism recited in claim 1 and the absence a complex indicates the absence of the microorganism recited in claim 1.

25. A method for identifying a microorganism in a sample from an individual, the method comprises:
a. labeling rRNA obtained from the sample to thereby obtain labeled rRNA;
b. contacting rRNA obtained from the sample with one or more nucleic acid molecules having a nucleic acid sequence selected from the group consisting of:
i. any one of SEQ ID NOs:1-295;
ii. a nucleic acid sequence having between about 80% and about 100% of contiguous nucleotides of any one of SEQ ID NO: 1-295;
iii. any one of SEQ ID NOs:296-585;
iv. a nucleic acid sequence having between about 15 and about 25 contiguous nucleotides of any one of SEQ ID NO: 296-585; and
v. any combination thereof;
under conditions suitable for hybridization to thereby form a complex; and
c. detecting the presence or absence of the complex;
wherein the presence of a complex indicates the presence of the microorganism recited in claim 1 and the absence a complex indicates the absence of the microorganism recited in claim 1.

26. The method of claim 25, wherein the label is attached to a universal probe, and the universal probe and the nucleic acid molecules hybridize to different portions of the RNA from the sample.

27. An array for the identification of one or more microorganisms, wherein the array comprises one or more nucleic acid molecules having a nucleic acid sequence selected from the group consisting of:
a. any one of SEQ ID NOs:1-295;
b. a nucleic acid sequence having between about 80% and about 100% of contiguous nucleotides of any one of SEQ ID NO: 1-295;
c. any one of SEQ ID NOs:296-585;
d. a nucleic acid sequence having between about 15 and about 25 contiguous nucleotides of any one of SEQ ID NO: 296-585;
e. a reverse complement of a-d; and
f. any combination thereof;
wherein each molecule is bound to the surface of a solid support in a different localized area.

28. The array of claim 27, wherein the solid support is selected from the group consisting of glass, silica chips, nylon membrane, polymer, plastic, ceramic, metal, and optical fiber.

29. The array of claim 27, wherein the solid support has between about 1 and about 8 different arrays.

30. The array of claim 29, wherein the solid support has about 5 different arrays.

31. The array of claim 27, wherein the same array is duplicated 2 or more times.

32. The array of claim 27, wherein the nucleic acid molecules having SEQ ID NOs: 1-585 are derived from 16S DNA sequence from the microorganism to be identified.

33. The array of claim 27, wherein one or more two nucleic acid molecules are used to identify one microorganism.

34. An array for the identification of one or more microorganisms, wherein the array comprises at least about 10% of the nucleic acid molecules having a nucleic acid sequence selected from the group consisting of:
a. any one of SEQ ID NOs:1-295;
b. a nucleic acid sequence having between about 80% and about 100% of contiguous nucleotides of any one of SEQ ID NO: 1-295;
c. any one of SEQ ID NOs:296-585;
d. a nucleic acid sequence having between about 15 and about 25 contiguous nucleotides of any one of SEQ ID NO: 296-585;
e. a reverse complement of a-d; and
f. any combination thereof;
wherein each sequence is bound to the surface of a solid support in a different localized area.

35. An array for the identification of one or more microorganisms, wherein the array comprises at least about 20% of the nucleic acid molecules having a nucleic acid sequence selected from the group consisting of:
a. any one of SEQ ID NOs:1-295;
b. a nucleic acid sequence having between about 80% and about 100% of contiguous nucleotides of any one of SEQ ID NO: 1-295;
c. any one of SEQ ID NOs:296-585;
d. a nucleic acid sequence having between about 15 and about 25 contiguous nucleotides of any one of SEQ ID NO: 296-585;
e. a reverse complement of a-d; and
f. any combination thereof;
wherein each sequence is bound to the surface of a solid support in a different localized area.

36. A kit that comprises:
a. one or more arrays for the identification of one or more microorganisms, wherein the array comprises one or more nucleic acid molecules having a nucleic acid sequence selected from the group consisting of:
i. any one of SEQ ID NOs:1-295;
ii. a nucleic acid sequence having between about 80% and about 100% of contiguous nucleotides of any one of SEQ ID NO: 1-295;
iii. any one of SEQ ID NOs:296-585;
iv. a nucleic acid sequence having between about 15 and about 25 contiguous nucleotides of any one of SEQ ID NO: 296-585;
v. a reverse complement of a-d; and
vi. any combination thereof;
wherein each sequence is bound to the surface of a solid support in a different localized area; and
b. one or more reagents used for carrying out a nucleic acid hybridization assay.

37. The kit of claim 36, wherein the regents include compounds used to detect hybridization; unlabeled primers, labeled primers, washing solutions; and buffers.

38. An isolated nucleic acid molecule from a bacterium isolated from a human oral cavity, sinus, esophagus, respiratory tract, lungs, pharynx, eustachian tube, or middle ear having a nucleic acid sequence selected from the group consisting of:
a. any one of SEQ ID NOs:1-295;
b. a nucleic acid sequence having between about 80% and about 100% of contiguous nucleotides of any one of SEQ ID NO: 1-295;
c. any one of SEQ ID NOs:296-585;
d. a nucleic acid sequence having between about 15 and about 25 contiguous nucleotides of any one of SEQ ID NO: 296-585;
e. a reverse complement of a-d; and
f. any combination thereof;
wherein the isolated nucleic acid molecule is used to identify one or more of the microorganisms recited in claim 1.

39. The isolated nucleic acid molecule of claim 38, wherein the nucleic acid molecule is an DNA or RNA molecule.

40. A probe for identifying one or more microorganisms, wherein the probe has a nucleic acid sequence selected from the group consisting of:
a. any one of SEQ ID NOs:1-295;
b. a nucleic acid sequence having between about 80% and about 100% of contiguous nucleotides of any one of SEQ ID NO: 1-295;
c. any one of SEQ ID NOs:296-585;
d. a nucleic acid sequence having between about 15 and about 25 contiguous nucleotides of any one of SEQ ID NO: 296-585;
e. a reverse complement of a-d; and
f. any combination thereof;
wherein the isolated nucleic acid molecule is used to identify one or more of the microorganisms recited in claim 1.

41. A method of making an array for the identification of a microorganism; the method comprises attaching to a solid support one or more nucleic acid molecules having a nucleic acid sequence selected from the group consisting of:
a. any one of SEQ ID NOs:1-295;
b. a nucleic acid sequence having between about 80% and about 100% of contiguous nucleotides of any one of SEQ ID NO: 1-295;
c. any one of SEQ ID NOs:296-585;
d. a nucleic acid sequence having between about 15 and about 25 contiguous nucleotides of any one of SEQ ID NO: 296-585;
e. a reverse complement of a-d; and
f. any combination thereof;
wherein each molecule is attached to the surface of a solid support in a different localized area.

42. The method of claim 41, wherein the nucleic acid molecules are from a solution having a concentration of between about 30 μM and 200 μM.

43. The method of claim 42, wherein the nucleic acid molecules are from a solution having a concentration of about 100 μM.

44. The method of claim 41, wherein between about 1 and about 8 arrays are printed on one glass slide.

45. The method of claim 44, wherein about 5 arrays are printed on one glass slide.

46. The method of claim 45, wherein the same array is duplicated 2 or more times.

47. A method of making an array for the identification of a microorganism; the method comprises:
a. synthesizing a capture probe having a nucleic acid sequence selected from the group consisting of:
i. any one of SEQ ID NOs:1-295;
ii. a nucleic acid sequence having between about 80% and about 100% of contiguous nucleotides of any one of SEQ ID NO: 1-295;
iii. any one of SEQ ID NOs:296-585;
iv. a nucleic acid sequence having between about 15 and about 25 contiguous nucleotides of any one of SEQ ID NO: 296-585;
v. a reverse complement of i-iv; and
vi. any combination thereof; and
b. attaching one or more nucleic acid molecules to a solid support, wherein each molecule is attached to the surface of a solid support in a different localized area.

48. The method of claim 47, wherein the step of synthesizing the capture probe further includes attaching a spacer to the probe.

49. The method of claim 48, wherein a spacer includes a plurality of thymidines.

50. The method of claim 48, wherein the step of synthesizing the capture probe further includes attaching a molecule to the probe that reacts with the solid support.

51. The method of claim 50, wherein a molecule that reacts with the solid support includes an amine group.

52. A method of making an array for the identification of a microorganism; the method comprises inserting or integrating within a solid support one or more nucleic acid molecules having a nucleic acid sequence selected from the group consisting of:
a. any one of SEQ ID NOs:1-295;
b. a nucleic acid sequence having between about 80% and about 100% of contiguous nucleotides of any one of SEQ ID NO: 1-295;
c. any one of SEQ ID NOs:296-585;
d. a nucleic acid sequence having between about 15 and about 25 contiguous nucleotides of any one of SEQ ID NO: 296-585;
e. a reverse complement of a-d; and
f. any combination thereof;
wherein each molecule is inserted within the surface of a solid support in a different localized area.


SEQUENCE LISTING (585 sequences)

SEQ ID NO: 1 - DNA, 19 bp, Actinobacillus actinomycetemcomitans
tctctgagttcttcttcgg

SEQ ID NO: 2 - DNA, 20 bp, Actinobacillus actinomycetemcomitans
accaaccttcctcaataccg

SEQ ID NO: 3 - DNA, 18 bp, Actinobaculum sp. EL030
gagttaagccccgcattt

SEQ ID NO: 4 - DNA, 17 bp, Actinobaculum sp. EL030
gaagaaggcagggtact

SEQ ID NO: 5 - DNA, 18 bp, Actinomyces georgiae
atgcgaggaccagtgaat

SEQ ID NO: 6 - DNA, 16 bp, Actinomyces georgiae
aaagccaggcttcagc

SEQ ID NO: 7 - DNA, 16 bp, Actinomyces gerensceriae
accccagaagcccgtt

SEQ ID NO: 8 - DNA, 18 bp, Actinomyces gerensceriae
acagacccagaaacatcc

SEQ ID NO: 9 - DNA, 17 bp, Actinomyces naeslundii I
agaccggcacatgtcaa

SEQ ID NO: 10 - DNA, 18 bp, Actinomyces naeslundii II
tcatccagaaccagcaag

SEQ ID NO: 11 - DNA, 17 bp, Actinomyces odontolyticus
acaaccttgagttgcgg

SEQ ID NO: 12 - DNA, 19 bp, Actinomyces odontolyticus
ccatgcgaagatcagtgaa

SEQ ID NO: 13 - DNA, 17 bp, Actinomyces sp. AP064
acacccaccacaaagga

SEQ ID NO: 14 - DNA, 17 bp, Actinomyces sp. strain B19SC
cagagccagaatatccc

SEQ ID NO: 15 - DNA, 18 bp, Actinomyces sp. strain B19SC
gaatcgccgacatgtcaa

SEQ ID NO: 16 - DNA, 18 bp, Actinomyces sp. strain B27SC
tgcggagaccagtgaata

SEQ ID NO: 17 - DNA, 14 bp, Actinomyces sp. strain B27SC
actcacccacccag

SEQ ID NO: 18 - DNA, 18 bp, Actinomyces sp. EP005
agcaaaaaccggtccctt

SEQ ID NO: 19 - DNA, 18 bp, Actinomyces sp. EP011
ccacaaaaaaggagcagg

SEQ ID NO: 20 - DNA, 16 bp, Actinomyces sp. EP011
cacaagggaggaaacc

SEQ ID NO: 21 - DNA, 18 bp, Actinomyces sp. EP053
cacttcacagtgtcgcaa

SEQ ID NO: 22 - DNA, 18 bp, Actinomyces sp. EP053
aaccggtcgaatctttcc

SEQ ID NO: 23 - DNA, 16 bp, Actinomyces israelii
ggcacagccagaacac

SEQ ID NO: 24 - DNA, 17 bp, Actinomyces israelii
ttcttcaccggcgaaga

SEQ ID NO: 25 - DNA, 18 bp, Atopobium parvulum
ggctatcccaatgaaagg

SEQ ID NO: 26 - DNA, 18 bp, Atopobium parvulum
gcggaagtctcgaagtat

SEQ ID NO: 27 - DNA, 18 bp, Atopobium rimae
gccacattgaagtatcgg

SEQ ID NO: 28 - DNA, 18 bp, Atopobium rimae
ggaagacgtattctccca

SEQ ID NO: 29 - DNA, 18 bp, Atopobium sp. C019
gcggctcaagtgaagtat

SEQ ID NO: 30 - DNA, 19 bp, Tannerella forsythia
gaagaaagctctcactctc

SEQ ID NO: 31 - DNA, 18 bp, Tannerella forsythia
ttgcgggcaggttacata

SEQ ID NO: 32 - DNA, 18 bp, Bacteroidetes sp. _X083
gcagtaccaacagagtac

SEQ ID NO: 33 - DNA, 18 bp, Bacteroidetes sp. _X083
gcccatacatttgacagc

SEQ ID NO: 34 - DNA, 19 bp, Bacteroidetes sp. AU126
agatgcctcttccgtttac

SEQ ID NO: 35 - DNA, 19 bp, Bacteroidetes sp. AU126
acgtgtctcactttactcc

SEQ ID NO: 36 - DNA, 14 bp, Bifidobacterium (Genus-specific)
ggacgcgaccccat

SEQ ID NO: 37 - DNA, 18 bp, Bifidobacterium dentium
ccataccgacggatcttt

SEQ ID NO: 38 - DNA, 18 bp, Bifidobacterium sp. CX010
ccccatcatatggcacaa

SEQ ID NO: 39 - DNA, 18 bp, Bifidobacterium sp. CX010
agctatccccaaccatac

SEQ ID NO: 40 - DNA, 17 bp, Bifidobacterium sp. strain A32ED
cggatcggtcgggaaca

SEQ ID NO: 41 - DNA, 20 bp, Brevundimonas diminuta
attccgaaccaaaaggcacg

SEQ ID NO: 42 - DNA, 20 bp, Brevundimonas diminuta
tgggtttccaggcatgtcaa

SEQ ID NO: 43 - DNA, 18 bp, Bulledia extructa/Solobacterium moorei
ccagagattatcccagtc

SEQ ID NO: 44 - DNA, 22 bp, Campylobacter concisus
ccctatctaacttatgtaagac

SEQ ID NO: 45 - DNA, 18 bp, Campylobacter concisus
ctgtggacggtaactaat

SEQ ID NO: 46 - DNA, 19 bp, Campylobacter gracilis
tatagtctcatcccttgcc

SEQ ID NO: 47 - DNA, 17 bp, Campylobacter gracilis
agcaaggggcagattag

SEQ ID NO: 48 - DNA, 19 bp, Campylobacter rectus/concisus
ctgttgtcctctagtgtag

SEQ ID NO: 49 - DNA, 20 bp, Campylobacter rectus/concisus
tatagcctcatcctacaccg

SEQ ID NO: 50 - DNA, 19 bp, Campylobacter cluster: (C.rectus/showae/curvus)
gtgcttattccttgggtac

SEQ ID NO: 51 - DNA, 18 bp, Campylobacter cluster: (C.rectus/showae/curvus)
cggtttggtatttgggct

SEQ ID NO: 52 - DNA, 18 bp, Campylobacter showae
agccctatccattaccga

SEQ ID NO: 53 - DNA, 19 bp, Campylobacter showae
gtaatgggcaagttagcta

SEQ ID NO: 54 - DNA, 18 bp, Capnocytophaga sp. _X066
actcaaactcgcgtccaa

SEQ ID NO: 55 - DNA, 18 bp, Capnocytophaga sp. _X089
ggtctcagtcactcgaaa

SEQ ID NO: 56 - DNA, 19 bp, Capnocytophaga sp. _X089
ttaggtaaccgtcttcagg

SEQ ID NO: 57 - DNA, 18 bp, Capnocytophaga sp. AA032
tcaaactacgcgttagcc

SEQ ID NO: 58 - DNA, 18 bp, Capnocytophaga sp. AA032
caacagttccaaaggcag

SEQ ID NO: 59 - DNA, 18 bp, Capnocytophaga sp. BB167
gccgtgctcttataccat

SEQ ID NO: 60 - DNA, 18 bp, Capnocytophaga sp. BB167
aataagtgcacgatgccg

SEQ ID NO: 61 - DNA, 20 bp, Capnocytophaga ochracea/sp. BB167
attagagggctcgaccttac

SEQ ID NO: 62 - DNA, 18 bp, Capnocytophaga Cluster I: (Capnocytophaga sp. BM058/BU084/DZ074)
ctaagtacgtcagtctcc

SEQ ID NO: 63 - DNA, 18 bp, Capnocytophaga Cluster II:  (C.ochracea/BM058/BU084/ DZ074/BR085)
ctcgctagcaactaatgg

SEQ ID NO: 64 - DNA, 18 bp, Capnocytophaga sp. BR085
tatctatcgtcgcctcgg

SEQ ID NO: 65 - DNA, 19 bp, Capnocytophaga sp. DS022
gcttattcacagagtaccg

SEQ ID NO: 66 - DNA, 19 bp, Capnocytophaga sp. DS022
tctaagtacgtcagtcacc

SEQ ID NO: 67 - DNA, 20 bp, Capnocytophaga gingivalis/S3
gccactcaacttatcatcaa

SEQ ID NO: 68 - DNA, 20 bp, Capnocytophaga sputigena
taaaaacgatgccgctccta

SEQ ID NO: 69 - DNA, 18 bp, Cardiobacterium hominis
aacgtcaattgcgcaggt

SEQ ID NO: 70 - DNA, 18 bp, Corynebacterium durum
agactgcaaacacacagc

SEQ ID NO: 71 - DNA, 18 bp, Corynebacterium matruchotii
ttccagcgcgttgtacta

SEQ ID NO: 72 - DNA, 19 bp, Cryptobacterium curtum
acatctctgtcacttcacc

SEQ ID NO: 73 - DNA, 19 bp, Desulfobulbus sp. _R004/CH031
agttattcgctgccttgca

SEQ ID NO: 74 - DNA, 18 bp, Dialister invisus
gcggctcttggaacttat

SEQ ID NO: 75 - DNA, 17 bp, Dialister invisus
tttctgcagatcgcgat

SEQ ID NO: 76 - DNA, 18 bp, Dialister pneumosintes
cctcttaaggcgatagct

SEQ ID NO: 77 - DNA, 18 bp, Dialister pneumosintes
tcaccacaaaccctttcg

SEQ ID NO: 78 - DNA, 18 bp, Eikenella corrodens
aataacgcgaggtcttgc

SEQ ID NO: 79 - DNA, 18 bp, Escherichia coli
agcacattctcatctctg

SEQ ID NO: 80 - DNA, 18 bp, Eubacterium brachy
ctcctaggttactgtcag

SEQ ID NO: 81 - DNA, 19 bp, Eubacterium infirmum
gtccgaagaagaatccgat

SEQ ID NO: 82 - DNA, 19 bp, Eubacterium nodatum
aaccgagctttcagtggga

SEQ ID NO: 83 - DNA, 18 bp, Eubacterium saphenum
ctgtccgaagaaataccc

SEQ ID NO: 84 - DNA, 18 bp, Eubacterium saphenum
cactcaagtctgccagtt

SEQ ID NO: 85 - DNA, 20 bp, Eubacterium sp. strain A3MT
gctttgctgtttctatctcc

SEQ ID NO: 86 - DNA, 18 bp, Eubacterium sp. BB124
gaagtctcctcggcaata

SEQ ID NO: 87 - DNA, 18 bp, Eubacterium sp. BB142
aagctcgtctataaccgc

SEQ ID NO: 88 - DNA, 18 bp, Eubacterium sp. DO008
gtcattcttccacccgaa

SEQ ID NO: 89 - DNA, 21 bp, Eubacterium sp. IR009
gacttccttttaacagcttcg

SEQ ID NO: 90 - DNA, 19 bp, Eubacterium sulci
gtgtatctcttgcgttatg

SEQ ID NO: 91 - DNA, 18 bp, Eubacterium yurii
cccaacagtagttgagct

SEQ ID NO: 92 - DNA, 18 bp, Eubacterium yurii
cgtgtgtcacaggttgat

SEQ ID NO: 93 - DNA, 17 bp, Filifactor alocis
aggctgtcattggtatg

SEQ ID NO: 94 - DNA, 20 bp, Filifactor alocis
cctgcactcaagttaaacag

SEQ ID NO: 95 - DNA, 18 bp, Fusobacterium Cluster: (F.nucleatum/naviforme/ss. vincentii/CZ006/_R002)
cacttcacagctttgcga

SEQ ID NO: 96 - DNA, 18 bp, Fusobacterium nucleatum ss. nucleatum
cttcccatcattgccttg

SEQ ID NO: 97 - DNA, 18 bp, Fusobacterium nucleatum ss. polymorphum
gttgtccctatctgtgag

SEQ ID NO: 98 - DNA, 18 bp, Fusobacterium periodonticum
gtcatgcagtttccaacg

SEQ ID NO: 99 - DNA, 23 bp, Fusobacterium sp. _I035
attctaagatgccttataatcat

SEQ ID NO: 100 - DNA, 19 bp, Fusobacterium sp. BS011
cataatcttaggatgccct

SEQ ID NO: 101 - DNA, 19 bp, Gemella haemolysans
ggtaccgtctctactgtgt

SEQ ID NO: 102 - DNA, 23 bp, Gemella morbillorum
taaatatctctcatgcgagaaat

SEQ ID NO: 103 - DNA, 18 bp, Granulicatella adiacens/elegans
tccatccatcagcagaag

SEQ ID NO: 104 - DNA, 17 bp, Haemophilus influenzae
acgggtgccagagttaa

SEQ ID NO: 105 - DNA, 18 bp, Haemophilus parainfluenzae/paraphrophilus
actaaatgccttcctcgc

SEQ ID NO: 106 - DNA, 18 bp, Haemophilus segnis
aaacatcacgccttcctc

SEQ ID NO: 107 - DNA, 18 bp, Haemophilus segnis
ctctctcctgttaccgtt

SEQ ID NO: 108 - DNA, 18 bp, Haemophilus paraphrophaemolyticus/BJ021
ggattgcttccctctgta

SEQ ID NO: 109 - DNA, 18 bp, Haemophilus sp. BJ095
atcgccaccctctgtata

SEQ ID NO: 110 - DNA, 18 bp, Haemophilus sp. BJ095
cactccttcctcaatacc

SEQ ID NO: 111 - DNA, 18 bp, Kingella denitrificans
cactactcggtacattcc

SEQ ID NO: 112 - DNA, 17 bp, Kingella denitrificans
ttggctgctcaaatagc

SEQ ID NO: 113 - DNA, 19 bp, Kingella oralis
gcttaccagttcaaaacgc

SEQ ID NO: 114 - DNA, 16 bp, Lactobacillus Cluster:  (L.casei/rhamnosus/zeae)
cagccaagaaccatgc

SEQ ID NO: 115 - DNA, 18 bp, Lactobacillus Cluster:  (L.casei/rhamnosus/zeae)
agttactctgccgaccat

SEQ ID NO: 116 - DNA, 19 bp, Lactobacillus fermentum
atcaatcaattgggccaac

SEQ ID NO: 117 - DNA, 19 bp, Lactobacillus fermentum
accgtcaacgtatgaacag

SEQ ID NO: 118 - DNA, 18 bp, Lactobacillus gasseri
ggtgttatcccagtctct

SEQ ID NO: 119 - DNA, 18 bp, Lactobacillus gasseri
ctagacatgcgtctagtg

SEQ ID NO: 120 - DNA, 18 bp, Lactobacillus vaginalis/CX036
ggcccatcatgaagtgat

SEQ ID NO: 121 - DNA, 18 bp, Lactobacillus vaginalis/CX036
acagttactctcacgcac

SEQ ID NO: 122 - DNA, 18 bp, Lactobacillus sp. HT070
tacgattggcgctagatg

SEQ ID NO: 123 - DNA, 18 bp, Lautropia mirabilis
acccccaaatctcttcag

SEQ ID NO: 124 - DNA, 18 bp, Lautropia mirabilis
tttcgttcccgccaaaag

SEQ ID NO: 125 - DNA, 19 bp, Lautropia sp. AP009
tctccaaggttccggacat

SEQ ID NO: 126 - DNA, 18 bp, Leptotrichia buccalis
tgatggcaactagcgata

SEQ ID NO: 127 - DNA, 18 bp, Leptotrichia hofstadii
tatcaggtgttgtcagtc

SEQ ID NO: 128 - DNA, 18 bp, Leptotrichia sp. DR011
ggtaactaagcaacaggg

SEQ ID NO: 129 - DNA, 17 bp, Leptotrichia sp. FB074/BB002
tatgacctcccggcgat

SEQ ID NO: 130 - DNA, 17 bp, Leptotrichia sp. GT018
ccacatagtttccaggg

SEQ ID NO: 131 - DNA, 18 bp, Leptotrichia wadei
ctctagccgcatagtttc

SEQ ID NO: 132 - DNA, 18 bp, Leptotrichia wadei
ctttgaaggttggcttgg

SEQ ID NO: 133 - DNA, 18 bp, Megasphaera sp. BB166
cgctaagaggaccgtatt

SEQ ID NO: 134 - DNA, 18 bp, Megasphaera sp. BB166
tcttcctcttaccatgcg

SEQ ID NO: 135 - DNA, 19 bp, Megasphaera sp. BU057
tctctactccttgcgatca

SEQ ID NO: 136 - DNA, 18 bp, Megasphaera sp. BU057
agtcatgcgactttcgga

SEQ ID NO: 137 - DNA, 18 bp, Megasphaera sp. CS025
tctacgcccttcactcaa

SEQ ID NO: 138 - DNA, 18 bp, Megasphaera sp. CS025
gatactctcagccaacca

SEQ ID NO: 139 - DNA, 18 bp, Micromonas micros
ttctgtggtctcatgcgg

SEQ ID NO: 140 - DNA, 18 bp, Micromonas Cluster:  (M.micros/FG014/BS044)
catgcgattctgtggtct

SEQ ID NO: 141 - DNA, 19 bp, Micromonas sp. DA014
attaatcgcggtttcccac

SEQ ID NO: 142 - DNA, 18 bp, Mycoplasma faucium
ctcccaccgactaatgat

SEQ ID NO: 143 - DNA, 19 bp, Mycoplasma hominis
gtaccgtcagtctgcaatc

SEQ ID NO: 144 - DNA, 18 bp, Mycoplasma salivarium
accgtcaatgtagaagca

SEQ ID NO: 145 - DNA, 18 bp, Neisseria elongata
tccgtctctgaaaggttc

SEQ ID NO: 146 - DNA, 18 bp, Neisseria elongata
ttatgagattggctccgc

SEQ ID NO: 147 - DNA, 18 bp, Neisseria flavescens
cgtcatcagctgtcgata

SEQ ID NO: 148 - DNA, 19 bp, Neisseria Cluster I:  (N.mucosa/sicca/flava/AP015)
tattaaccccgcccttttc

SEQ ID NO: 149 - DNA, 18 bp, Neisseria pharyngis
gggtattaaccctgtcct

SEQ ID NO: 150 - DNA, 18 bp, Neisseria Cluster II:  (N.polysaccharea/gonorrhoeae/ meningitidis)
caaccgaatgatggcaac

SEQ ID NO: 151 - DNA, 19 bp, Neisseria Cluster II:  (N.polysaccharea/gonorrhoeae/ meningitidis)
tttccctctcaagacgtat

SEQ ID NO: 152 - DNA, 18 bp, Neisseria bacilliformis/AP132
gaataacgcgaggtccta

SEQ ID NO: 153 - DNA, 16 bp, Neisseria Cluster III:  (N.elongata/AP015/Eikenella corrodens
gaaggttccgtacatg

SEQ ID NO: 154 - DNA, 17 bp, Neisseria mucosa/AP060
gtcagcacgcaatggta

SEQ ID NO: 155 - DNA, 18 bp, Neisseria sp. B33KA
gtcatcagctcctggtat

SEQ ID NO: 156 - DNA, 17 bp, Olsenella genomospecies C1
catctgagtgtcaagcc

SEQ ID NO: 157 - DNA, 18 bp, Olsenella genomospecies C1
taactctcgacctactgg

SEQ ID NO: 158 - DNA, 18 bp, Peptostreptococcus sp. CK035
attaaacacctgtccgag

SEQ ID NO: 159 - DNA, 18 bp, Porphyromonas catoniae
catttcctctagcctgga

SEQ ID NO: 160 - DNA, 18 bp, Porphyromonas catoniae
ttcaccggtggtcatcta

SEQ ID NO: 161 - DNA, 19 bp, Porphyromonas endodontalis Cluster: (P. endodontalis/ F016/BB134/AJ002)
aagcaaactctcatctgcc

SEQ ID NO: 162 - DNA, 18 bp, Porphyromonas endodontalis Cluster:  (P. endodontalis/ F016/BB134/AJ002)
tccctaattacaggcagg

SEQ ID NO: 163 - DNA, 18 bp, Porphyromonas gingivalis
tcagtcgcagtatggcaa

SEQ ID NO: 164 - DNA, 18 bp, Porphyromonas gingivalis
gtggaagcttgacggtat

SEQ ID NO: 165 - DNA, 18 bp, Porphyromonas sp. BB134
ctttccgtctttccccat

SEQ ID NO: 166 - DNA, 18 bp, Porphyromonas sp. BB134
tttccgtctttccccatg

SEQ ID NO: 167 - DNA, 18 bp, Porphyromonas Cluster: (Porphyromonas sp. BR037/ DP023/EP003)
atgccctatcccaagtgt

SEQ ID NO: 168 - DNA, 18 bp, Porphyromonas sp. CW034/DS033
atgcccaaagtggagtgt

SEQ ID NO: 169 - DNA, 18 bp, Porphyromonas sp. CW034/DS033
ttggatgcccaaagtgga

SEQ ID NO: 170 - DNA, 17 bp, Porphyromonas sp. DP023
cttggagtaggatgccc

SEQ ID NO: 171 - DNA, 19 bp, Porphyromonas sp. DP023
tttccttggagtaggatgc

SEQ ID NO: 172 - DNA, 18 bp, Prevotella buccae
tctctgaatcattctcct

SEQ ID NO: 173 - DNA, 18 bp, Prevotella buccae
cacgtgggcaactttatc

SEQ ID NO: 174 - DNA, 18 bp, Prevotella (Bacteroides) heparinolytica
caaggcacccagtatcaa

SEQ ID NO: 175 - DNA, 17 bp, Prevotella (Bacteroides) heparinolytica
ccggataattcggttgc

SEQ ID NO: 176 - DNA, 18 bp, Prevotella intermedia
cgctttactccccaacaa

SEQ ID NO: 177 - DNA, 19 bp, Prevotella loeschii/GU027
gagcacccggatttcacaa

SEQ ID NO: 178 - DNA, 21 bp, Prevotella Cluster:  (P.loeschii/GU027/strain B31FD)
atgccaccgactctctgtatg

SEQ ID NO: 179 - DNA, 18 bp, Prevotella melaninogenica
cacacttaatctccagcc

SEQ ID NO: 180 - DNA, 18 bp, Prevotella nigrescens
gctgcgactgcaattcaa

SEQ ID NO: 181 - DNA, 18 bp, Prevotella nigrescens
gtttcatatcggatgccg

SEQ ID NO: 182 - DNA, 18 bp, Prevotella oralis
ttcggactgcatacggaa

SEQ ID NO: 183 - DNA, 18 bp, Prevotella oralis
accgcatttacatacggc

SEQ ID NO: 184 - DNA, 16 bp, Prevotella oris/_F045
tcagtcagaggcaggt

SEQ ID NO: 185 - DNA, 18 bp, Prevotella oulora
agctctcgtcatcgtttc

SEQ ID NO: 186 - DNA, 18 bp, Prevotella oulora
ctctaatgagccaacagc

SEQ ID NO: 187 - DNA, 18 bp, Prevotella pallens
ataacgcacacgtgcatc

SEQ ID NO: 188 - DNA, 18 bp, Prevotella denticola/sp. AH005/AO036
catcgaagaacatgcggt

SEQ ID NO: 189 - DNA, 18 bp, Prevotella denticola/sp. AH005
tctgtatcgttctcctgc

SEQ ID NO: 190 - DNA, 18 bp, Prevotella sp. AH125
cgcgtgtccctctttatt

SEQ ID NO: 191 - DNA, 18 bp, Prevotella sp. AH125
acgcgtgtccctctttat

SEQ ID NO: 192 - DNA, 18 bp, Prevotella sp. BE073
ccgtcagtgaagaccata

SEQ ID NO: 193 - DNA, 18 bp, Prevotella sp. BI027
ctacctactacgcactca

SEQ ID NO: 194 - DNA, 18 bp, Prevotella sp. BI027
gatcgaagtcttggtgag

SEQ ID NO: 195 - DNA, 18 bp, Prevotella sp. CY006/FL019
cgttcacccttttatccc

SEQ ID NO: 196 - DNA, 21 bp, Prevotella sp. DO022
agaccccgaagggcgtattta

SEQ ID NO: 197 - DNA, 18 bp, Prevotella sp. DO027
cccaagcttaacctgatg

SEQ ID NO: 198 - DNA, 19 bp, Prevotella sp. DO027
gatgccatcaaaggattac

SEQ ID NO: 199 - DNA, 19 bp, Prevotella sp. DO039
gcgagaaagcaacaacatc

SEQ ID NO: 200 - DNA, 17 bp, Prevotella sp. DO039
cggcattgaaagcaagc

SEQ ID NO: 201 - DNA, 18 bp, Prevotella sp. DO045
acatgcaacctgccttct

SEQ ID NO: 202 - DNA, 18 bp, Prevotella sp. DO045
cacaacacgcttagtcca

SEQ ID NO: 203 - DNA, 19 bp, Prevotella sp. FM005
cacagtaatgggtaggttg

SEQ ID NO: 204 - DNA, 18 bp, Prevotella sp. HF050
ccaagagtgtccgaagaa

SEQ ID NO: 205 - DNA, 18 bp, Prevotella tannerae
ctgcataacagagttggg

SEQ ID NO: 206 - DNA, 18 bp, Prevotella tannerae
cagctgacttatactccc

SEQ ID NO: 207 - DNA, 18 bp, Propionibacterium acnes
tctctgagcactcccgat

SEQ ID NO: 208 - DNA, 20 bp, Propionibacterium sp. strain FMA5
tgagcgcttccagtacatgt

SEQ ID NO: 209 - DNA, 18 bp, Pseudomonas aeruginosa
ccactaccaggcagattc

SEQ ID NO: 210 - DNA, 18 bp, Rhodocyclus sp. strain A08KA
ccgtattagagagtgcga

SEQ ID NO: 211 - DNA, 19 bp, Rothia dentocariosa
tgacgcagtccagtatatg

SEQ ID NO: 212 - DNA, 18 bp, Rothia dentocariosa
ctgacgcagtccagtata

SEQ ID NO: 213 - DNA, 18 bp, Rothia dentocariosa
cgcagtccagtatatgtc

SEQ ID NO: 214 - DNA, 17 bp, Rothia dentocariosa/mucilaginosa
gcggagattggtcgtat

SEQ ID NO: 215 - DNA, 18 bp, Selenomonas dianae
gaggatgctatctctagc

SEQ ID NO: 216 - DNA, 18 bp, Selenomonas flueggii
accgtcattgcatgacac

SEQ ID NO: 217 - DNA, 18 bp, Selenomonas infelix
attcacccttcgcacgtt

SEQ ID NO: 218 - DNA, 20 bp, Selenomonas noxia
gtaccgtcattacctaatac

SEQ ID NO: 219 - DNA, 18 bp, Selenomonas sp. AA024
cttatgtacgttcgtccc

SEQ ID NO: 220 - DNA, 20 bp, Selenomonas sp. AA024
ggtaccgtcattacataagc

SEQ ID NO: 221 - DNA, 18 bp, Selenomonas sp. AH132
tgacactgttcgcatcac

SEQ ID NO: 222 - DNA, 18 bp, Selenomonas sp. AJ036
ttgaggttcgctcaacct

SEQ ID NO: 223 - DNA, 17 bp, Selenomonas sp. AJ036
tcgaaggaggatgccct

SEQ ID NO: 224 - DNA, 18 bp, Selenomonas sp. CI002
ctttcatgcaggggagat

SEQ ID NO: 225 - DNA, 17 bp, Selenomonas sp. CI002
ctcccctgcacttcatt

SEQ ID NO: 226 - DNA, 18 bp, Selenomonas sp. CS002
ggcgcaacattcggtatt

SEQ ID NO: 227 - DNA, 18 bp, Selenomonas sp. CS002
aagatggacttgcatgcc

SEQ ID NO: 228 - DNA, 18 bp, Selenomonas sp. CS015
tttcctccccttcccttt

SEQ ID NO: 229 - DNA, 19 bp, Selenomonas sp. CS015
gtaccgtcttaaaaagggc

SEQ ID NO: 230 - DNA, 18 bp, Selenomonas sp. CS024
gcactatacacgttcgtc

SEQ ID NO: 231 - DNA, 16 bp, Selenomonas sp. CS024
agatgcctccctcgca

SEQ ID NO: 232 - DNA, 17 bp, Selenomonas sp. DD020
aacccggttttcgtccc

SEQ ID NO: 233 - DNA, 18 bp, Selenomonas sp. DM071
cccatgtgagagagttac

SEQ ID NO: 234 - DNA, 18 bp, Selenomonas sp. EZ011
aacgcattgcctcgtcta

SEQ ID NO: 235 - DNA, 16 bp, Selenomonas sp. DS051
gaagatgcctccccta

SEQ ID NO: 236 - DNA, 18 bp, Selenomonas sp. DS051
attcgcactgcgtacgtt

SEQ ID NO: 237 - DNA, 18 bp, Selenomonas sp. EW076
ctctgcatgcttcagtca

SEQ ID NO: 238 - DNA, 17 bp, Selenomonas sp. EW076
tttgaggttcgcctacc

SEQ ID NO: 239 - DNA, 18 bp, Selenomonas sp. EW079/JS031
gtttctgtcccttacagg

SEQ ID NO: 240 - DNA, 18 bp, Selenomonas sp. EW084/DS071
ccgtcattacagagcact

SEQ ID NO: 241 - DNA, 16 bp, Selenomonas sp. EW084/DS071
cactccgtacgttcgt

SEQ ID NO: 242 - DNA, 18 bp, Selenomonas sputigena
ccgtcacccaaactcaat

SEQ ID NO: 243 - DNA, 18 bp, Selenomonas sputigena
tctcaagctcggttttcg

SEQ ID NO: 244 - DNA, 17 bp, Streptococcus (Genus-specific)
agccgtccctttctggt

SEQ ID NO: 245 - DNA, 21 bp, Streptococcus anginosus/intermedius
attctcacacttgttcttcct

SEQ ID NO: 246 - DNA, 22 bp, Streptococcus anginosus/gordonii
caactcacagtctatggtgtag

SEQ ID NO: 247 - DNA, 22 bp, Streptococcus constellatus/intermedius
cagtaaatgttcttatgcggta

SEQ ID NO: 248 - DNA, 17 bp, Streptococcus constellatus/intermedius
acatctaccatgcagta

SEQ ID NO: 249 - DNA, 21 bp, Streptococcus cristatus
catgcaatagtcaatgttatg

SEQ ID NO: 250 - DNA, 18 bp, Streptococcus cristatus
ctcatccagaagagcaag

SEQ ID NO: 251 - DNA, 18 bp, Streptococcus Cluster I:  (S.gordonii/anginosus/mitis)
agtctatggtgtagcaag

SEQ ID NO: 252 - DNA, 20 bp, Streptococcus mitis biovar 2
caayaactgctattatgcgg

SEQ ID NO: 253 - DNA, 21 bp, Streptococcus Cluster II:   (S.mitis/oralis/pneumoniae)
ccttttaagyaaatgtcatgc

SEQ ID NO: 254 - DNA, 18 bp, Streptococcus mutans
tttactccagactttcct

SEQ ID NO: 255 - DNA, 21 bp, Streptococcus parasanguinis
gtcgacttttatgcggtatta

SEQ ID NO: 256 - DNA, 19 bp, Streptococcus parasanguinis
gtcaacatcatgcaatgtc

SEQ ID NO: 257 - DNA, 19 bp, Streptococcus salivarius
gtcatccattgttatgcgg

SEQ ID NO: 258 - DNA, 18 bp, Streptococcus salivarius
gacatgggtcatccattg

SEQ ID NO: 259 - DNA, 19 bp, Streptococcus Cluster III:  (S.sanguinis/salivarius/ mitis/C3)
tgcaactcatccaagaaga

SEQ ID NO: 260 - DNA, 18 bp, Streptococcus australis
atccggaaagagcaagct

SEQ ID NO: 261 - DNA, 22 bp, Streptococcus sp. C6/C3/P4/7A
atgcgataatccattttatgcg

SEQ ID NO: 262 - DNA, 20 bp, Streptococcus infantis/FN042
gcacctttcaagcagctatc

SEQ ID NO: 263 - DNA, 19 bp, Stretococcus sobrinus
gttaactcctcttatgcgg

SEQ ID NO: 264 - DNA, 19 bp, Stretococcus sobrinus
taacatgagttaactcctc

SEQ ID NO: 265 - DNA, 21 bp, All Synergistes (Genus-specific)
tccttttacagctgacttgaa

SEQ ID NO: 266 - DNA, 17 bp, Synergistes sp. _D084
acacgagtgcctcctgt

SEQ ID NO: 267 - DNA, 18 bp, Synergistes sp. _W028
ggctcctacctcatacat

SEQ ID NO: 268 - DNA, 18 bp, Synergistes sp. _W090
cagactcctaccacatac

SEQ ID NO: 269 - DNA, 21 bp, Synergistes sp. BB062
cagactcttaccacgtacatg

SEQ ID NO: 270 - DNA, 18 bp, Synergistes sp. BH017
tcagtctcagccgcataa

SEQ ID NO: 271 - DNA, 19 bp, Tannerella sp. BU063
tctgttgtaggtaggttgc

SEQ ID NO: 272 - DNA, 18 bp, TM7 sp. _I025
cgaacaacaagctatcgg

SEQ ID NO: 273 - DNA, 18 bp, TM7 sp. AH040
ggcactaattggtttccc

SEQ ID NO: 274 - DNA, 17 bp, TM7 sp. BE109
actttggggcatgttcc

SEQ ID NO: 275 - DNA, 18 bp, TM7 sp. BE109/BU080
gagtttgccagttcgaat

SEQ ID NO: 276 - DNA, 18 bp, Treponema 08:A:pectinovorum
caccactataccattccc

SEQ ID NO: 277 - DNA, 18 bp, Treponema 08:A:pectinovorum
cgcttcactctgttccaa

SEQ ID NO: 278 - DNA, 18 bp, Treponema (Genus-specific)
taacyggcagtaggggtt

SEQ ID NO: 279 - DNA, 19 bp, Treponema denticola
ctaccgtcatcaaagaagc

SEQ ID NO: 280 - DNA, 22 bp, Treponema lecithinolyticum
tacgaacttaagtactatgtca

SEQ ID NO: 281 - DNA, 18 bp, Treponema lecithinolyticum
ttgctcatccgcctacat

SEQ ID NO: 282 - DNA, 18 bp, Treponema medium
cccttatgaagcactgag

SEQ ID NO: 283 - DNA, 19 bp, Treponema socranskii (all sub-species)
ctctatatgatccctcttg

SEQ ID NO: 284 - DNA, 24 bp, Treponema sp. AT039
cttatgaaatattgagtgtattcg

SEQ ID NO: 285 - DNA, 19 bp, Treponema vincentii
ctctaagactgtctactag

SEQ ID NO: 286 - DNA, 18 bp, Veillonella dispar/_X042
tgagttcccacccaaagt

SEQ ID NO: 287 - DNA, 18 bp, Veillonella dispar/_X042
tctctggttctgtccatc

SEQ ID NO: 288 - DNA, 18 bp, Veillonella (Genus-specific)
aatcccctccttcagtga

SEQ ID NO: 289 - DNA, 18 bp, Veillonella atypica
cctttcatccagtctcga

SEQ ID NO: 290 - DNA, 18 bp, Veillonella atypica
tcgcacaagaaccattcg

SEQ ID NO: 291 - DNA, 17 bp, Veillonella parvula
atctcgcgatctcgctt

SEQ ID NO: 292 - DNA, 18 bp, Veillonella sp. AA050/_X042
ttgcaagaaggcctttcg

SEQ ID NO: 293 - DNA, 20 bp, Veillonella sp. AA050/_X042
caatccttctcactatttgc

SEQ ID NO: 294 - DNA, 18 bp, Veillonella sp. BU083
cacctttcatccatcctc

SEQ ID NO: 295 - DNA, 18 bp, Bacterial Universal
ttcatccatcctcgatgc

SEQ ID NO: 296 - DNA, 43 bp, Actinobacillus actinomycetemcomitans
ggcacaaacccatctctgagttcttcttcggatgtcaagagta

SEQ ID NO: 297 - DNA, 44 bp, Actinobacillus actinomycetemcomitans
tgctattaacacaccaaccttcctcaataccgaaagaactttac

SEQ ID NO: 298 - DNA, 42 bp, Actinobaculum sp. EL030
actgcacgtacggagttaagccccgcattttcacagcagacg

SEQ ID NO: 299 - DNA, 41 bp, Actinobaculum sp. EL030
ggcttatccaaagaagaaggcagggtactcacgtattactc

SEQ ID NO: 300 - DNA, 42 bp, Actinomyces georgiae
ccaccaacccccatgcgaggaccagtgaatacccggtattag

SEQ ID NO: 301 - DNA, 40 bp, Actinomyces georgiae
tccacccaacacaaagccaggcttcagcgttcgacttgca

SEQ ID NO: 302 - DNA, 40 bp, Actinomyces gerensceriae
aaaaaagccagaaccccagaagcccgttcgacttgcatgt

SEQ ID NO: 303 - DNA, 42 bp, Actinomyces gerensceriae
acccgccatgcgacagacccagaaacatcccgtattagccac

SEQ ID NO: 304 - DNA, 41 bp, Actinomyces naeslundii I
cgtctccggagcagaccggcacatgtcaagccttggtaagg

SEQ ID NO: 305 - DNA, 42 bp, Actinomyces naeslundii II
cccgttcgccactcatccagaaccagcaagctggctccttca

SEQ ID NO: 306 - DNA, 41 bp, Actinomyces odontolyticus
actaccctcaccacaaccttgagttgcggcttgaccatgag

SEQ ID NO: 307 - DNA, 43 bp, Actinomyces odontolyticus
ttccaccaacccccatgcgaagatcagtgaatatccagtatta

SEQ ID NO: 308 - DNA, 41 bp, Actinomyces sp. AP064
cgccactcatccacacccaccacaaaggaaggcgcttcacc

SEQ ID NO: 309 - DNA, 41 bp, Actinomyces sp. strain B19SC
cccaccatgcgacagagccagaatatcccgtattaacacca

SEQ ID NO: 310 - DNA, 42 bp, Actinomyces sp. strain B19SC
gcgtctccgcgggaatcgccgacatgtcaagccttggtaagg

SEQ ID NO: 311 - DNA, 42 bp, Actinomyces sp. strain B27SC
caccagaccccatgcggagaccagtgaatacccggtattagc

SEQ ID NO: 312 - DNA, 38 bp, Actinomyces sp. strain B27SC
cacccgttcgccactcacccacccagaaacccagacga

SEQ ID NO: 313 - DNA, 42 bp, Actinomyces sp. EP005
tcatccagaaccagcaaaaaccggtcccttcaccgttcgact

SEQ ID NO: 314 - DNA, 42 bp, Actinomyces sp. EP011
ccgtcaacccacccacaaaaaaggagcaggcctgcttcactg

SEQ ID NO: 315 - DNA, 40 bp, Actinomyces sp. EP011
tgaaccagcccccacaagggaggaaaccccgtctccggag

SEQ ID NO: 316 - DNA, 42 bp, Actinomyces sp. EP053
agagattagcttcacttcacagtgtcgcaacccgttgtaccg

SEQ ID NO: 317 - DNA, 42 bp, Actinomyces sp. EP053
caaaaacaccaaaaccggtcgaatctttccaaacccacccat

SEQ ID NO: 318 - DNA, 40 bp, Actinomyces israelii
gccacaccatgcggcacagccagaacaccccgtattagcc

SEQ ID NO: 319 - DNA, 41 bp, Actinomyces israelii
aagagcaggaccttcttcaccggcgaagaggttcacaaccc

SEQ ID NO: 320 - DNA, 42 bp, Atopobium parvulum
acggtttcccgtggctatcccaatgaaaggggcaggttgccc

SEQ ID NO: 321 - DNA, 42 bp, Atopobium parvulum
cgagtcttccatgcggaagtctcgaagtattcggtattatca

SEQ ID NO: 322 - DNA, 42 bp, Atopobium rimae
tgtcgtcatgcggccacattgaagtatcgggtattatcctcg

SEQ ID NO: 323 - DNA, 42 bp, Atopobium rimae
tagctgcggcacggaagacgtattctcccacacctagtgtcc

SEQ ID NO: 324 - DNA, 42 bp, Atopobium sp. C019
cacttgagtcatgcggctcaagtgaagtatcgggtattatcc

SEQ ID NO: 325 - DNA, 43 bp, Tannerella forsythia
gacgccccgaaggaagaaagctctcactctccgtcgtctacat

SEQ ID NO: 326 - DNA, 42 bp, Tannerella forsythia
gttatccctctgttgcgggcaggttacatacgcgttactcac

SEQ ID NO: 327 - DNA, 42 bp, Bacteroidetes sp. _X083
catcttacgatggcagtaccaacagagtacacgactaatttc

SEQ ID NO: 328 - DNA, 42 bp, Bacteroidetes sp. _X083
aataagggttgagcccatacatttgacagctgacttaaaaaa

SEQ ID NO: 329 - DNA, 43 bp, Bacteroidetes sp. AU126
ttaaaacaaaaaagatgcctcttccgtttactatggggtatta

SEQ ID NO: 330 - DNA, 43 bp, Bacteroidetes sp. AU126
gcaataaaacacacgtgtctcactttactcctgtacaaaagaa

SEQ ID NO: 331 - DNA, 42 bp, Bifidobacterium dentium
cgcgaccccatcccataccgacggatctttcccggaaggaca

SEQ ID NO: 332 - DNA, 42 bp, Bifidobacterium sp. CX010
gataggacgcgaccccatcatatggcacaaaaagctttccca

SEQ ID NO: 333 - DNA, 42 bp, Bifidobacterium sp. CX010
cccgtttccaggagctatccccaaccatacggcaggttagtc

SEQ ID NO: 334 - DNA, 41 bp, Bifidobacterium sp. strain A32ED
aagacccgtctccggatcggtcgggaacatgtcaagcccag

SEQ ID NO: 335 - DNA, 44 bp, Brevundimonas diminuta
tttccctgagttattccgaaccaaaaggcacgttcccacgtgtt

SEQ ID NO: 336 - DNA, 44 bp, Brevundimonas diminuta
gccacgtctccgtgggtttccaggcatgtcaaaaggtggtaagg

SEQ ID NO: 337 - DNA, 42 bp, Bulledia extructa/Solobacterium moorei
tagtccccgtttccagagattatcccagtcttcgtgggtagg

SEQ ID NO: 338 - DNA, 46 bp, Campylobacter concisus
ccgaaaaactttccctatctaacttatgtaagacaggagtatagag

SEQ ID NO: 339 - DNA, 42 bp, Campylobacter concisus
tcgctgattccactgtggacggtaactaatttagtattccgg

SEQ ID NO: 340 - DNA, 43 bp, Campylobacter gracilis
tagctgatacgatatagtctcatcccttgccgaaattctttcc

SEQ ID NO: 341 - DNA, 41 bp, Campylobacter gracilis
ctgttgtcctccagcaaggggcagattagctatatattact

SEQ ID NO: 342 - DNA, 43 bp, Campylobacter rectus/concisus
agtcgtttccaactgttgtcctctagtgtagggcagattagct

SEQ ID NO: 343 - DNA, 25 bp, Campylobacter rectus/concisus
atatagcctcatcctacaccgaaaa

SEQ ID NO: 344 - DNA, 36 bp, Campylobacter cluster: (C.rectus/showae/curvus)
cggagttagccggtgcttattccttgggtaccgtca

SEQ ID NO: 345 - DNA, 42 bp, Campylobacter cluster: (C.rectus/showae/curvus)
gtggaccataaccggtttggtatttgggcttcgagtgaaatc

SEQ ID NO: 346 - DNA, 42 bp, Campylobacter showae
ctgatacgatatagccctatccattaccgaaaaactttcccg

SEQ ID NO: 347 - DNA, 43 bp, Campylobacter showae
tgttgtcccttagtaatgggcaagttagctatatattactcac

SEQ ID NO: 348 - DNA, 42 bp, Capnocytophaga sp. _X066
tttcgcttagccactcaaactcgcgtccaaacagctagtatc

SEQ ID NO: 349 - DNA, 42 bp, Capnocytophaga sp. _X089
cacccgtacgccggtctcagtcactcgaaagtaacctccccc

SEQ ID NO: 350 - DNA, 43 bp, Capnocytophaga sp. _X089
taaacagctcctttaggtaaccgtcttcaggtactcccagctt

SEQ ID NO: 351 - DNA, 41 bp, Capnocytophaga sp. AA032
aaggtaccgtcatcaaactacgcgttagcccttattcttcc

SEQ ID NO: 352 - DNA, 42 bp, Capnocytophaga sp. AA032
accattcaagaccaacagttccaaaggcagttgctcagttga

SEQ ID NO: 353 - DNA, 42 bp, Capnocytophaga sp. BB167
taagtgcacgatgccgtgctcttataccatagggtattaatc

SEQ ID NO: 354 - DNA, 42 bp, Capnocytophaga sp. BB167
cgccgtagctttaataagtgcacgatgccgtgctcttatacc

SEQ ID NO: 355 - DNA, 44 bp, Capnocytophaga ochracea/sp. BB167
accggcagtcccattagagggctcgaccttactcgttagcaact

SEQ ID NO: 356 - DNA, 42 bp, Capnocytophaga Cluster I: (Capnocytophaga sp. BM058/ BU084/DZ074)
aaatacctatctctaagtacgtcagtctccatttaaaccttg

SEQ ID NO: 357 - DNA, 42 bp, Capnocytophaga Cluster II:  (C.ochracea/BM058/BU084/ DZ074/BR085)
tgctcgactcgactcgctagcaactaatggcaggggttgcgc

SEQ ID NO: 358 - DNA, 42 bp, Capnocytophaga sp. BR085
tctcagaacccctatctatcgtcgcctcggggagccgttacc

SEQ ID NO: 359 - DNA, 43 bp, Capnocytophaga sp. DS022
gagttagccgatgcttattcacagagtaccgtcatcaaactac

SEQ ID NO: 360 - DNA, 43 bp, Capnocytophaga sp. DS022
gaaatacctatctctaagtacgtcagtcaccatttaaaccttg

SEQ ID NO: 361 - DNA, 44 bp, Capnocytophaga gingivalis/S3
cactttcgcttagccactcaacttatcatcaaacagctagtatc

SEQ ID NO: 362 - DNA, 44 bp, Capnocytophaga sputigena
gtagctttaatataaaaacgatgccgctcctatataccattagg

SEQ ID NO: 363 - DNA, 42 bp, Cardiobacterium hominis
tcttctgtaggtaacgtcaattgcgcaggtattaactacgca

SEQ ID NO: 364 - DNA, 42 bp, Corynebacterium durum
tagctacggcacagactgcaaacacacagcccacacctagcg

SEQ ID NO: 365 - DNA, 42 bp, Corynebacterium matruchotii
caccctcacaggttccagcgcgttgtactaaccatgtagcat

SEQ ID NO: 366 - DNA, 43 bp, Cryptobacterium curtum
ctttcggctgcgacatctctgtcacttcacctacatgtcaagc

SEQ ID NO: 367 - DNA, 43 bp, Desulfobulbus sp. _R004/CH031
gtcaaacaaaacagttattcgctgccttgcacttcttccctct

SEQ ID NO: 368 - DNA, 41 bp, Dialister invisus
ccatgggccatgcggctcttggaacttattcggtattagca

SEQ ID NO: 369 - DNA, 41 bp, Dialister invisus
aagaactccgcatttctgcagatcgcgatcaatgtcaagac

SEQ ID NO: 370 - DNA, 42 bp, Dialister pneumosintes
cagtcgcaaacccctcttaaggcgatagctttcttgtagagg

SEQ ID NO: 371 - DNA, 42 bp, Dialister pneumosintes
gtctatgattattcaccacaaaccctttcgtcccgaatcaca

SEQ ID NO: 372 - DNA, 42 bp, Eikenella corrodens
atcggccgctcgaataacgcgaggtcttgcgatcccccgctt

SEQ ID NO: 373 - DNA, 42 bp, Escherichia coli
tcggttcccggaagcacattctcatctctgaaaacacttccg

SEQ ID NO: 374 - DNA, 42 bp, Eubacterium brachy
agccggggctttctcctaggttactgtcagttttcatcacct

SEQ ID NO: 375 - DNA, 43 bp, Eubacterium infirmum
cctgtctctcttgtccgaagaagaatccgattaaggatctgtc

SEQ ID NO: 376 - DNA, 43 bp, Eubacterium nodatum
gggctcagttttaaccgagctttcagtgggatgtcaagtcctg

SEQ ID NO: 377 - DNA, 42 bp, Eubacterium saphenum
cacctgtctcctctgtccgaagaaatacccgattaagggtac

SEQ ID NO: 378 - DNA, 42 bp, Eubacterium saphenum
tttcctctcttgcactcaagtctgccagttcgcaaggctaac

SEQ ID NO: 379 - DNA, 44 bp, Eubacterium sp. strain A3MT
gtgaccgctttcgctttgctgtttctatctcccaatttaaacag

SEQ ID NO: 380 - DNA, 42 bp, Eubacterium sp. BB124
tatgatttgaccgaagtctcctcggcaataattctccgttcg

SEQ ID NO: 381 - DNA, 42 bp, Eubacterium sp. BB124
ctaatcagacgcaagctcgtctataaccgcctcagctttgat

SEQ ID NO: 382 - DNA, 42 bp, Eubacterium sp. DO008
gccactcagtctgtcattcttccacccgaaggtctccaaaga

SEQ ID NO: 383 - DNA, 45 bp, Eubacterium sp. IR009
attccatccgaagacttccttttaacagcttcgttcgacttgcat

SEQ ID NO: 384 - DNA, 43 bp, Eubacterium sulci
caagtgatacatgtgtatctcttgcgttatggggtattaatca

SEQ ID NO: 385 - DNA, 41 bp, Eubacterium yurii
ttagtttcaaggcccaacagtagttgagctactgcctttta

SEQ ID NO: 386 - DNA, 42 bp, Eubacterium yurii
ctatcagttatccgtgtgtcacaggttgattacttacgcgtt

SEQ ID NO: 387 - DNA, 41 bp, Filifactor alocis
tcctcgattaaaaggctgtcattggtatgtcaagtttaggt

SEQ ID NO: 388 - DNA, 44 bp, Filifactor alocis
ccactttcctctcctgcactcaagttaaacagttttaatggctt

SEQ ID NO: 389 - DNA, 42 bp, Fusobacterium Cluster: (F.nucleatum/naviforme/ss. vincentii/CZ006/_R002)
tgagattagctccacttcacagctttgcgactctctgttcta

SEQ ID NO: 390 - DNA, 42 bp, Fusobacterium nucleatum ss. nucleatum
ctctcaggccggcttcccatcattgccttggtgagccgttac

SEQ ID NO: 391 - DNA, 42 bp, Fusobacterium nucleatum ss. polymorphum
tcgtttccaaatgttgtccctatctgtgaggcaagttcttta

SEQ ID NO: 392 - DNA, 42 bp, Fusobacterium periodonticum
tccagtactctagtcatgcagtttccaacgcaatacagagtt

SEQ ID NO: 393 - DNA, 47 bp, Fusobacterium sp. _I035
atagctttcataattctaagatgccttataatcataatatcaggtat

SEQ ID NO: 394 - DNA, 43 bp, Fusobacterium sp. BS011
gtatataactttcataatcttaggatgccctaaaatcataata

SEQ ID NO: 395 - DNA, 43 bp, Gemella haemolysans
gctttctggttaggtaccgtctctactgtgtatagttactaca

SEQ ID NO: 396 - DNA, 47 bp, Gemella morbillorum
ataaccaacttttaaatatctctcatgcgagaaatactgttatccgg

SEQ ID NO: 397 - DNA, 42 bp, Granulicatella adiacens/elegans
atgcaccgcgggtccatccatcagcagaagccgaagcctctt

SEQ ID NO: 398 - DNA, 41 bp, Haemophilus influenzae
atcacgttagctacgggtgccagagttaaaccccaaccccc

SEQ ID NO: 399 - DNA, 42 bp, Haemophilus parainfluenzae/paraphrophilus
ctagtctattaaactaaatgccttcctcgctaccgaaagaac

SEQ ID NO: 400 - DNA, 42 bp, Haemophilus segnis
gtgatgcctattaaacatcacgccttcctcgtcaccgaaaga

SEQ ID NO: 401 - DNA, 42 bp, Haemophilus segnis
caagaaagcaagctctctcctgttaccgttcgacttgcatgt

SEQ ID NO: 402 - DNA, 42 bp, Haemophilus paraphrophaemolyticus/BJ021
ctccccctcgcaggattgcttccctctgtatacgccattgta

SEQ ID NO: 403 - DNA, 42 bp, Haemophilus sp. BJ095
ccacctcgcggcatcgccaccctctgtatacgccattgtagc

SEQ ID NO: 404 - DNA, 42 bp, Haemophilus sp. BJ095
gctattaacatacactccttcctcaataccgaaagaacttta

SEQ ID NO: 405 - DNA, 42 bp, Kingella denitrificans
ttggttatcccccactactcggtacattccaatatgttactc

SEQ ID NO: 406 - DNA, 41 bp, Kingella denitrificans
gctaatcagatattggctgctcaaatagcgcaaggtccgaa

SEQ ID NO: 407 - DNA, 43 bp, Kingella oralis
tgacacactctagcttaccagttcaaaacgcagttcccaagtt

SEQ ID NO: 408 - DNA, 40 bp, Lactobacillus Cluster:  (L.casei/rhamnosus/zeae)
trcgccatctttcagccaagaaccatgcggttcttggaty

SEQ ID NO: 409 - DNA, 42 bp, Lactobacillus Cluster:  (L.casei/rhamnosus/zeae)
cacgccgacaacagttactctgccgaccattcttctccaaca

SEQ ID NO: 410 - DNA, 43 bp, Lactobacillus fermentum
ggtgcaagcaccatcaatcaattgggccaacgcgttcgactag

SEQ ID NO: 411 - DNA, 43 bp, Lactobacillus fermentum
ttctggttaaataccgtcaacgtatgaacagttactctcatac

SEQ ID NO: 412 - DNA, 42 bp, Lactobacillus gasseri
catctgtttccaggtgttatcccagtctcttgggcaggttac

SEQ ID NO: 413 - DNA, 42 bp, Lactobacillus gasseri
atcttttaaactctagacatgcgtctagtgttgttatccggt

SEQ ID NO: 414 - DNA, 42 bp, Lactobacillus vaginalis/CX036
taatgcaccgcgggcccatcatgaagtgatagccgaaaccat

SEQ ID NO: 415 - DNA, 42 bp, Lactobacillus vaginalis/CX036
gtcactgcgtgaacagttactctcacgcacgttcttctccaa

SEQ ID NO: 416 - DNA, 42 bp, Lactobacillus sp. HT070
acatcgtatctctacgattggcgctagatgtcaagacctggt

SEQ ID NO: 417 - DNA, 42 bp, Lautropia mirabilis
tctctttcgagcacccccaaatctcttcagggttccggacat

SEQ ID NO: 418 - DNA, 42 bp, Lautropia mirabilis
tggcacagtccttttcgttcccgccaaaagtgctttacaacc

SEQ ID NO: 419 - DNA, 43 bp, Lautropia sp. AP009
caccctcgaatctctccaaggttccggacatgtcaagcgtagg

SEQ ID NO: 420 - DNA, 42 bp, Leptotrichia buccalis
tccccaacttaatgatggcaactagcgataggggttgcgctc

SEQ ID NO: 421 - DNA, 42 bp, Leptotrichia hofstadii
tgccggcaactgtatcaggtgttgtcagtcgtttccgtctgt

SEQ ID NO: 422 - DNA, 42 bp, Leptotrichia sp. DR011
caacttaatgatggtaactaagcaacaggggttgcgctcgtt

SEQ ID NO: 423 - DNA, 41 bp, Leptotrichia sp. FB074/BB002
cagtcatacttctatgacctcccggcgatgtcaaggtctgg

SEQ ID NO: 424 - DNA, 41 bp, Leptotrichia sp. GT018
ccagtactctagccacatagtttccagggcaggcttgcggt

SEQ ID NO: 425 - DNA, 42 bp, Leptotrichia wadei
acctctccagtactctagccgcatagtttccagggcaggctt

SEQ ID NO: 426 - DNA, 42 bp, Leptotrichia wadei
aacttggaccggctttgaaggttggcttggcgttgccgcctt

SEQ ID NO: 427 - DNA, 42 bp, Megasphaera sp. BB166
cctcttaccatgcgctaagaggaccgtattcggtattagcag

SEQ ID NO: 428 - DNA, 42 bp, Megasphaera sp. BB166
gaggccaccctttcttcctcttaccatgcgctaagaggaccg

SEQ ID NO: 429 - DNA, 43 bp, Megasphaera sp. BU057
aagaacagagtatctctactccttgcgatcaatgtcaaggctt

SEQ ID NO: 430 - DNA, 42 bp, Megasphaera sp. BU057
ctttcttccgacagtcatgcgactttcggaacgtattcggta

SEQ ID NO: 431 - DNA, 42 bp, Megasphaera sp. CS025
gaacggcatatctctacgcccttcactcaatgtcaaggcttg

SEQ ID NO: 432 - DNA, 42 bp, Megasphaera sp. CS025
gctttcctctccgatactctcagccaaccagtttctctcccc

SEQ ID NO: 433 - DNA, 42 bp, Micromonas micros
tatatcatgcgattctgtggtctcatgcggtattaatcgtcg

SEQ ID NO: 434 - DNA, 42 bp, Micromonas Cluster:  (M.micros/FG014/BS044)
tgacccctatatcatgcgattctgtggtctcatgcggtatta

SEQ ID NO: 435 - DNA, 43 bp, Micromonas sp. DA014
gtcttatgaggtattaatcgcggtttcccacggctatccctct

SEQ ID NO: 436 - DNA, 42 bp, Mycoplasma faucium
ctgcgtcagtggctcccaccgactaatgatcatcgtttacgg

SEQ ID NO: 437 - DNA, 43 bp, Mycoplasma hominis
ctttctgacaaggtaccgtcagtctgcaatcatttcctattgc

SEQ ID NO: 438 - DNA, 42 bp, Mycoplasma salivarium
ttctaacaaggtaccgtcaatgtagaagcatttcctcaacta

SEQ ID NO: 439 - DNA, 42 bp, Neisseria elongata
cgaaggcacccttccgtctctgaaaggttccgtacatgtcaa

SEQ ID NO: 440 - DNA, 42 bp, Neisseria elongata
ctacgatcggttttatgagattggctccgcctcgcggcttgg

SEQ ID NO: 441 - DNA, 42 bp, Neisseria flavescens
ttcttcaggtaccgtcatcagctgtcgatattagcaacagcc

SEQ ID NO: 442 - DNA, 43 bp, Neisseria Cluster I:  (N.mucosa/sicca/flava/AP015)
atcagacaggggtattaaccccgcccttttcttccctgacaaa

SEQ ID NO: 443 - DNA, 42 bp, Neisseria pharyngis
gtcatcagacaggggtattaaccctgtccttttcttccctga

SEQ ID NO: 444 - DNA, 42 bp, Neisseria Cluster II:  (N.polysaccharea/gonorrhoeae/ meningitidis)
cattagagtgcccaaccgaatgatggcaactaatgacaaggg

SEQ ID NO: 445 - DNA, 43 bp, Neisseria Cluster II:  (N.polysaccharea/gonorrhoeae/ meningitidis)
aaggtcccctgctttccctctcaagacgtatgcggtattagct

SEQ ID NO: 446 - DNA, 42 bp, Neisseria bacilliformis/AP132
catcggccgctcgaataacgcgaggtcctaagatcccccgct

SEQ ID NO: 447 - DNA, 40 bp, Neisseria Cluster III:  (N.elongata/AP015/Eikenella corrodens
cttccgtctcyggaaggttccgtacatgtcaagaccaggt

SEQ ID NO: 448 - DNA, 41 bp, Neisseria mucosa/AP060
tcttccggtaccgtcagcacgcaatggtattaacatcgcgc

SEQ ID NO: 449 - DNA, 42 bp, Neisseria sp. B33KA
tcttcaggtaccgtcatcagctcctggtattatcaaaagcct

SEQ ID NO: 450 - DNA, 41 bp, Olsenella genomospecies C1
tttccgccgcttcatctgagtgtcaagccctggtaaggttc

SEQ ID NO: 451 - DNA, 42 bp, Olsenella genomospecies C1
agcccccggatttaactctcgacctactgggcagcctacgcg

SEQ ID NO: 452 - DNA, 42 bp, Peptostreptococcus sp. CK035
aggaagggtgtgattaaacacctgtccgagggatgtcaagct

SEQ ID NO: 453 - DNA, 42 bp, Porphyromonas catoniae
  misc_feature (38)..(38): n is a, c, g, or t
cacagcgaatttcatttcctctagcctggatgcccagnctgg

SEQ ID NO: 454 - DNA, 42 bp, Porphyromonas catoniae
  misc_feature (6)..(7): n is a, c, g, or t
gggaanncgactttcaccggtggtcatctacatttcaatccc

SEQ ID NO: 455 - DNA, 31 bp, Porphyromonas endodontalis Cluster: (P. endodontalis/ F016/BB134/AJ002)
tgtcccgaaggaaagcaaactctcatctgcc

SEQ ID NO: 456 - DNA, 42 bp, Porphyromonas endodontalis Cluster:  (P. endodontalis/ F016/BB134/AJ002)
tcaccgggttattccctaattacaggcaggttgcatacgcgt

SEQ ID NO: 457 - DNA, 42 bp, Porphyromonas gingivalis
cgtgcttcagtgtcagtcgcagtatggcaagctgccttcgca

SEQ ID NO: 458 - DNA, 42 bp, Porphyromonas gingivalis
aacgctttcgctgtggaagcttgacggtatatcgcaaactcc

SEQ ID NO: 459 - DNA, 42 bp, Porphyromonas sp. BB134
taatcaataaatctttccgtctttccccatgcggaaaaagaa

SEQ ID NO: 460 - DNA, 42 bp, Porphyromonas sp. BB134
aatcaataaatctttccgtctttccccatgcggaaaaagaag

SEQ ID NO: 461 - DNA, 42 bp, Porphyromonas Cluster: (Porphyromonas sp. BR037/ DP023/EP003)
ccttggatkaggatgccctatcccaagtgtacgcggtattag

SEQ ID NO: 462 - DNA, 42 bp, Porphyromonas sp. CW034/DS033
cctctactytggatgcccaaagtggagtgtacgcggtattag

SEQ ID NO: 463 - DNA, 42 bp, Porphyromonas sp. CW034/DS033
atttcctctactttggatgcccaaagtggagtgtacgcggta

SEQ ID NO: 464 - DNA, 41 bp, Porphyromonas sp. DP023
gaatttcatttccttggagtaggatgccctatcccaagtgt

SEQ ID NO: 465 - DNA, 43 bp, Porphyromonas sp. DP023
cagcgaatttcatttccttggagtaggatgccctatcccaagt

SEQ ID NO: 466 - DNA, 42 bp, Prevotella buccae
gggacttcatcatctctgaatcattctcctgcaattcaagcc

SEQ ID NO: 467 - DNA, 42 bp, Prevotella buccae
  misc_feature (7)..(7): n is a, c, g, or t
tgcaaanacccacacgtgggcaactttatccccgcataaaag

SEQ ID NO: 468 - DNA, 42 bp, Prevotella (Bacteroides) heparinolytica
ctatactgcactcaaggcacccagtatcaactgcaattttaa

SEQ ID NO: 469 - DNA, 41 bp, Prevotella (Bacteroides) heparinolytica
gcttacccgtttccggataattcggttgcaattcaagcccg

SEQ ID NO: 470 - DNA, 42 bp, Prevotella intermedia
ggcacacgtgcccgctttactccccaacaaaagcagtttaca

SEQ ID NO: 471 - DNA, 43 bp, Prevotella loeschii/GU027
aagccggatgttgagcacccggatttcacaacacgcttaaggc

SEQ ID NO: 472 - DNA, 44 bp, Prevotella Cluster:  (P.loeschii/GU027/strain B31FD)
gatggcaytcgcatgccaccgactctctgtatgcgccattgtaa

SEQ ID NO: 473 - DNA, 42 bp, Prevotella melaninogenica
tacatttcacaacacacttaatctccagcctacgctcccttt

SEQ ID NO: 474 - DNA, 42 bp, Prevotella nigrescens
acgtctctgtgggctgcgactgcaattcaagcccgggtaagg

SEQ ID NO: 475 - DNA, 42 bp, Prevotella nigrescens
ggaaaacctttggtttcatatcggatgccgtcaatgaaacac

SEQ ID NO: 476 - DNA, 42 bp, Prevotella oralis
ttagatgactgcttcggactgcatacggaattaggccgcctt

SEQ ID NO: 477 - DNA, 42 bp, Prevotella oralis
tttaaggattggaccgcatttacatacggccgactcgctgta

SEQ ID NO: 478 - DNA, 40 bp, Prevotella oris/_F045
acgggttatccctcagtcagaggcaggttggatacgcgtt

SEQ ID NO: 479 - DNA, 42 bp, Prevotella oulora
cctaagagagcaagctctcgtcatcgtttcccctcgacttgc

SEQ ID NO: 480 - DNA, 42 bp, Prevotella oulora
cttggccgctgactctaatgagccaacagcgggcatccagcg

SEQ ID NO: 481 - DNA, 42 bp, Prevotella pallens
acgatacatgcaataacgcacacgtgcatcaaattattctcg

SEQ ID NO: 482 - DNA, 42 bp, Prevotella denticola/sp. AH005/AO036
atctgatgccgtcatcgaagaacatgcggtattagtctgcct

SEQ ID NO: 483 - DNA, 42 bp, Prevotella denticola/sp. AH005
gacctcaacatctctgtatcgttctcctgcaattcaagcccg

SEQ ID NO: 484 - DNA, 42 bp, Prevotella sp. AH125
tgcaacaggacacgcgtgtccctctttattccccttaaaaag

SEQ ID NO: 485 - DNA, 42 bp, Prevotella sp. AH125
atgcaacaggacacgcgtgtccctctttattccccttaaaaa

SEQ ID NO: 486 - DNA, 42 bp, Prevotella sp. BE073
tcacatctgatgccgtcagtgaagaccataaggtattagtct

SEQ ID NO: 487 - DNA, 42 bp, Prevotella sp. BI027
cacgaattccgcctacctactacgcactcaagttcaacagtt

SEQ ID NO: 488 - DNA, 42 bp, Prevotella sp. BI027
agaacccctactgatcgaagtcttggtgagccgttacctcac

SEQ ID NO: 489 - DNA, 42 bp, Prevotella sp. CY006/FL019
caaaggagaacacgttcacccttttatccccgtataaaagca

SEQ ID NO: 490 - DNA, 45 bp, Prevotella sp. DO022
gcaccttcacagagaccccgaagggcgtatttatctctaaataat

SEQ ID NO: 491 - DNA, 42 bp, Prevotella sp. DO027
gtctccagagtgcccaagcttaacctgatggcaactgaagag

SEQ ID NO: 492 - DNA, 43 bp, Prevotella sp. DO027
ttatccttatctgatgccatcaaaggattacataaggtattag

SEQ ID NO: 493 - DNA, 43 bp, Prevotella sp. DO039
ttactctcccatgcgagaaagcaacaacatcgggtattaatct

SEQ ID NO: 494 - DNA, 41 bp, Prevotella sp. DO039
tgcgccggtcgccggcattgaaagcaagctttcaacccgct

SEQ ID NO: 495 - DNA, 42 bp, Prevotella sp. DO045
tcgatcgcatttacatgcaacctgccttctgtaccggccatt

SEQ ID NO: 496 - DNA, 42 bp, Prevotella sp. DO045
gcatctacatttcacaacacgcttagtccacggcctacgctc

SEQ ID NO: 497 - DNA, 43 bp, Prevotella sp. FM005
ggcaggttatcccacagtaatgggtaggttggatacgcgttac

SEQ ID NO: 498 - DNA, 42 bp, Prevotella sp. HF050
catgcagcacctccaagagtgtccgaagaaaaatctatctct

SEQ ID NO: 499 - DNA, 42 bp, Prevotella tannerae
atatagtttcaactgcataacagagttgggctctgatattta

SEQ ID NO: 500 - DNA, 42 bp, Prevotella tannerae
tctgatatttaacagctgacttatactcccgcctgcgctccc

SEQ ID NO: 501 - DNA, 42 bp, Prevotella tannerae
gaggcacacccatctctgagcactcccgatccatgtcaaacc

SEQ ID NO: 502 - DNA, 44 bp, Propionibacterium sp. strain FMA5
cacgcccatctctgagcgcttccagtacatgtcaaacccaggta

SEQ ID NO: 503 - DNA, 42 bp, Pseudomonas aeruginosa
ggacgttatcccccactaccaggcagattcctaggcattact

SEQ ID NO: 504 - DNA, 42 bp, Rhodocyclus sp. strain A08KA
gtcatccacaccccgtattagagagtgcgatttcttcccagc

SEQ ID NO: 505 - DNA, 43 bp, Rothia dentocariosa
aaacgccatctctgacgcagtccagtatatgtcaagccttggt

SEQ ID NO: 506 - DNA, 42 bp, Rothia dentocariosa
gaaacgccatctctgacgcagtccagtatatgtcaagccttg

SEQ ID NO: 507 - DNA, 17 bp, Rothia dentocariosa
ctgctgcctcccgtagg

SEQ ID NO: 508 - DNA, 41 bp, Rothia dentocariosa/mucilaginosa
ccaacaccccatgcggagattggtcgtatccggtattagac

SEQ ID NO: 509 - DNA, 42 bp, Selenomonas dianae
tgtctccgaagagaggatgctatctctagcactttcagtcaa

SEQ ID NO: 510 - DNA, 42 bp, Selenomonas flueggii
tcctcgacaggtaccgtcattgcatgacactgttcgcatcac

SEQ ID NO: 511 - DNA, 42 bp, Selenomonas infelix
ttgcaaaggattattcacccttcgcacgttcgtccccatcaa

SEQ ID NO: 512 - DNA, 44 bp, Selenomonas noxia
cttcctcgatgggtaccgtcattacctaatactattcgcattag

SEQ ID NO: 513 - DNA, 42 bp, Selenomonas sp. AA024
aagcttattcaacttatgtacgttcgtccccttcaacagagc

SEQ ID NO: 514 - DNA, 44 bp, Selenomonas sp. AA024
gcttcctcaaaaggtaccgtcattacataagcttattcaactta

SEQ ID NO: 515 - DNA, 42 bp, Selenomonas sp. AH132
accgtcattgcatgacactgttcgcatcacgcacgttcgtcc

SEQ ID NO: 516 - DNA, 42 bp, Selenomonas sp. AJ036
gggaaacggtttttgaggttcgctcaacctcgcgggttcgct

SEQ ID NO: 517 - DNA, 41 bp, Selenomonas sp. AJ036
ggccatctttcatcgaaggaggatgccctccctcgacttca

SEQ ID NO: 518 - DNA, 42 bp, Selenomonas sp. CI002
aaaagaggccatctttcatgcaggggagatgcctcccctgca

SEQ ID NO: 519 - DNA, 41 bp, Selenomonas sp. CI002
caggggagatgcctcccctgcacttcattcggtattagcat

SEQ ID NO: 520 - DNA, 42 bp, Selenomonas sp. CS002
ccatgcgggaggggcgcaacattcggtattagcagccctttc

SEQ ID NO: 521 - DNA, 42 bp, Selenomonas sp. CS002
ccccgcgcttttaagatggacttgcatgcccgcctgcgctcc

SEQ ID NO: 522 - DNA, 42 bp, Selenomonas sp. CS015
ttaaaaagggcatttcctccccttccctttcgtccccaacaa

SEQ ID NO: 523 - DNA, 43 bp, Selenomonas sp. CS015
cttgctcgtcgggtaccgtcttaaaaagggcatttcctcccct

SEQ ID NO: 524 - DNA, 42 bp, Selenomonas sp. CS024
atagcactatttgcactatacacgttcgtccccttcaacaga

SEQ ID NO: 525 - DNA, 40 bp, Selenomonas sp. CS024
ttcatgcgggggagatgcctccctcgcacaccattcggta

SEQ ID NO: 526 - DNA, 41 bp, Selenomonas sp. DD020
tcaatattctcaaacccggttttcgtcccgtgcaacagagc

SEQ ID NO: 527 - DNA, 42 bp, Selenomonas sp. DM071
tttagtatctctcccatgtgagagagttacgtcattcggtat

SEQ ID NO: 528 - DNA, 42 bp, Selenomonas sp. EZ011
accgtcttagtaaacgcattgcctcgtctaccctttcgtccc

SEQ ID NO: 529 - DNA, 40 bp, Selenomonas sp. DS051
ctttcatgtagggaagatgcctcccctacacgccattcgg

SEQ ID NO: 530 - DNA, 42 bp, Selenomonas sp. DS051
ttacacagcactattcgcactgcgtacgttcgtccccttcaa

SEQ ID NO: 531 - DNA, 42 bp, Selenomonas sp. EW076
agacacatacatctctgcatgcttcagtcaatgtcaaggcct

SEQ ID NO: 532 - DNA, 41 bp, Selenomonas sp. EW076
tgggaaacggtttttgaggttcgcctaccctcgcgggttcg

SEQ ID NO: 533 - DNA, 42 bp, Selenomonas sp. EW079/JS031
tcaagaacgatagtttctgtcccttacaggggctgagccctt

SEQ ID NO: 534 - DNA, 42 bp, Selenomonas sp. EW084/DS071
cctcgayaggtaccgtcattacagagcactattcgcactccg

SEQ ID NO: 535 - DNA, 40 bp, Selenomonas sp. EW084/DS071
gagcactattcgcactccgtacgttcgtccccrtcaacag

SEQ ID NO: 536 - DNA, 42 bp, Selenomonas sputigena
cctcgctcggtaccgtcacccaaactcaatattctcaagctc

SEQ ID NO: 537 - DNA, 42 bp, Selenomonas sputigena
caaactcaatattctcaagctcggttttcgtcccgtgcaaca

SEQ ID NO: 538 - DNA, 40 bp, Streptococcus anginosus/intermedius
atgaactttccattctcacactcgttcttccttaacaaca

SEQ ID NO: 539 - DNA, 46 bp, Streptococcus anginosus/gordonii
ctcacccgttcgcaactcacagtctatggtgtagcaagctacggta

SEQ ID NO: 540 - DNA, 46 bp, Streptococcus constellatus/intermedius
acatctaccatgcagtaaatgttcttatgcggtattagctatcgtt

SEQ ID NO: 541 - DNA, 41 bp, Streptococcus constellatus/intermedius
tgcaccttttaaacatctaccatgcagtaaatgttcttatg

SEQ ID NO: 542 - DNA, 45 bp, Streptococcus cristatus
ttaattgactatcatgcaatagtcaatgttatgcggtattagcta

SEQ ID NO: 543 - DNA, 42 bp, Streptococcus cristatus
acccgttcgcaactcatccagaagagcaagctcctccttcag

SEQ ID NO: 544 - DNA, 42 bp, Streptococcus Cluster I:  (S.gordonii/anginosus/mitis)
ttcgcaactcacagtctatggtgtagcaagctacggtataaa

SEQ ID NO: 545 - DNA, 44 bp, Streptococcus mitis biovar 2
acagttatcatgcaayaactgctattatgcggtattagctatcg

SEQ ID NO: 546 - DNA, 45 bp, Streptococcus Cluster II:   (S.mitis/oralis/pneumoniae)
gatgcaattgcaccttttaagyaaatgtcatgcaacatctactct

SEQ ID NO: 547 - DNA, 42 bp, Streptococcus mutans
gagccatagccttttactccagactttcctgaccgcctgcgc

SEQ ID NO: 548 - DNA, 45 bp, Streptococcus parasanguinis
acatcatgcaatgtcgacttttatgcggtattagctatcgtttcc

SEQ ID NO: 549 - DNA, 43 bp, Streptococcus parasanguinis
tgcacctttcaagtcaacatcatgcaatgtcgacttttatgcg

SEQ ID NO: 550 - DNA, 43 bp, Streptococcus salivarius
taaatgacatgggtcatccattgttatgcggtattagctatcg

SEQ ID NO: 551 - DNA, 42 bp, Streptococcus salivarius
tttcaaataaatgacatgggtcatccattgttatgcggtatt

SEQ ID NO: 552 - DNA, 43 bp, Streptococcus Cluster III:  (S.sanguinis/salivarius/ mitis/C3)
tactcacccgttcgcaactcatccaagaagagcaagctcctct

SEQ ID NO: 553 - DNA, 42 bp, Streptococcus australis
cgttcgcaactcatccggaaagagcaagctccttccttcagc

SEQ ID NO: 554 - DNA, 45 bp, Streptococcus sp. C6/C3/P4/7A
caatggattatcatgcgataatccattttatgcggtattagctat

SEQ ID NO: 555 - DNA, 44 bp, Streptococcus infantis/FN042
agtggtgcaattgcacctttcaagcagctatcatgcgatatcta

SEQ ID NO: 556 - DNA, 43 bp, Stretococcus sobrinus
cagttaacatgagttaactcctcttatgcggtattagctatcg

SEQ ID NO: 557 - DNA, 43 bp, Stretococcus sobrinus
cttttaaacagttaacatgagttaactcctcttatgcggtatt

SEQ ID NO: 558 - DNA, 41 bp, Synergistes sp. _D084
ccagctgataggacacgagtgcctcctgtcgcgcatctctg

SEQ ID NO: 559 - DNA, 42 bp, Synergistes sp. _W028
gtcaccgtttccggctcctacctcatacatgtcaaacccagg

SEQ ID NO: 560 - DNA, 42 bp, Synergistes sp. _W090
tcgtcgccctttcagactcctaccacatacatgtcaaaccca

SEQ ID NO: 561 - DNA, 45 bp, Synergistes sp. BB062
tcatcatcctttcagactcttaccacgtacatgtcaaacccaggt

SEQ ID NO: 562 - DNA, 42 bp, Synergistes sp. BH017
tactccagcacctcagtctcagccgcataacacggttaagcc

SEQ ID NO: 563 - DNA, 43 bp, Tannerella sp. BU063
ccggattattcttctgttgtaggtaggttgcatacgcgttact

SEQ ID NO: 564 - DNA, 42 bp, TM7 sp. _I025
agtcaagcagttcgaacaacaagctatcggttgagccgatag

SEQ ID NO: 565 - DNA, 42 bp, TM7 sp. AH040
agggttccaaaaggcactaattggtttcccaaaaattccctg

SEQ ID NO: 566 - DNA, 41 bp, TM7 sp. BE109
cagttatccctcactttggggcatgttcccacgcgttactc

SEQ ID NO: 567 - DNA, 42 bp, TM7 sp. BE109/BU080
tctgctactctcgagtttgccagttcgaataatagtctgtat

SEQ ID NO: 568 - DNA, 42 bp, Treponema 08:A:pectinovorum
attacctactgtcacctctataccattccctgtacagtttat

SEQ ID NO: 569 - DNA, 42 bp, Treponema 08:A:pectinovorum
acgtcaccgcttcgcttcactctgttccaaccattgtagcac

SEQ ID NO: 570 - DNA, 43 bp, Treponema denticola
ttattcgcatgactaccgtcatcaaagaagcattccctcttct

SEQ ID NO: 571 - DNA, 46 bp, Treponema lecithinolyticum
catccgtatctctacgaacttaagtactatgtcaaacccaggtaag

SEQ ID NO: 572 - DNA, 42 bp, Treponema lecithinolyticum
ttcacaccaagcttgctcatccgcctacatgccctttacgcc

SEQ ID NO: 573 - DNA, 42 bp, Treponema medium
tttcctcaataccccttatgaagcactgagtgtattcggtat

SEQ ID NO: 574 - DNA, 43 bp, Treponema socranskii (all sub-species)
acgctcacatatctctatatgatccctcttgatgtcaaaccca

SEQ ID NO: 575 - DNA, 48 bp, Treponema sp. AT039
tcctcaatacttcttatgaaatattgagtgtattcggtattatctgct

SEQ ID NO: 576 - DNA, 43 bp, Treponema vincentii
accaagccttatctctaagactgtctactagatgtcaaaccca

SEQ ID NO: 577 - DNA, 42 bp, Veillonella dispar/_X042
ggcagtctctcatgagttcccacccaaagtgctggcaacata

SEQ ID NO: 578 - DNA, 42 bp, Veillonella dispar/_X042
gaagaggaaccatctctggttctgtccatcaatgtcaagacc

SEQ ID NO: 579 - DNA, 42 bp, Veillonella atypica
aaatagaggccacctttcatccagtctcgatgccgagattgg

SEQ ID NO: 580 - DNA, 42 bp, Veillonella atypica
tcctcgcactattcgcacaagaaccattcgtcccgattaaca

SEQ ID NO: 581 - DNA, 41 bp, Veillonella parvula
cgggtttgctccatctcgcgatctcgcttccgtctattaac

SEQ ID NO: 582 - DNA, 42 bp, Veillonella sp. AA050/_X042
ccttctcactatttgcaagaaggcctttcgtcccgattaaca

SEQ ID NO: 583 - DNA, 44 bp, Veillonella sp. AA050/_X042
ttccggtaccgtcaatccttctcactatttgcaagaaggccttt

SEQ ID NO: 584 - DNA, 42 bp, Veillonella sp. BU083
atacatagaggccacctttcatccatcctcgatgccgaggtt

SEQ ID NO: 585 - DNA, 42 bp, Veillonella sp. BU083
tagaggccacctttcatccatcctcgatgccgaggttagatc